(19) United States
(12) Patent Application Publication
(10) Pub. No.: US 2023/0117803 A1
(43) Pub. Date: Apr. 20, 2023

(54) TREATMENT INVOLVING ANTIGEN VACCINATION AND BINDING AGENTS BINDING TO PD-L1 AND CD137

(71) Applicant: BIONTECH SE, Mainz (DE)

(72) Inventors: Ugur SAHIN, Mainz (DE); Alexander MUIK, Mainz (DE); Lena KRANZ, Mainz (DE); Mathias VORMEHR, Mainz (DE)

(21) Appl. No.: 17/759,906

(22) PCT Filed: Feb. 2, 2021
(86) PCT No.: PCT/EP2021/052439

§ 371 Date: Aug. 1, 2022

(30) Foreign Application Priority Data
Feb. 4, 2020 (EP) PCT/EP2020/052774

(51) Int. Cl. C07K 16/28; A61K 39/00; A61P 35/00; A61P 37/04; A61P 17/00; A61K 39/395

(52) U.S. Cl. CPC C07K 16/2878; C07K 16/2827; A61K 39/001156; A61P 35/00; A61P 37/04; A61P 17/00; A61K 39/39558; A61K 2039/505; C07K 2317/76; C07K 2317/31; A61K 2039/545; A61K 2039/53; A61K 2039/55516

(57) ABSTRACT

The present disclosure relates to methods and compositions for inducing an immune response in a subject comprising providing to the subject a peptide or protein vaccine and a binding agent, such as a bispecific antibody, binding to PD-L1 and CD137, such as human PD-L1 and human CD137, e.g., by co-administering to the subject a peptide or protein used for vaccination or a polynucleotide, in particular RNA, encoding a peptide or protein used for vaccination, and a binding agent binding to PD-L1 and CD137 or a polynucleotide, in particular RNA, encoding a binding agent binding to PD-L1 and CD137. The present disclosure further relates to medical preparations useful in the methods disclosed herein.

### TECHNICAL FIELD

The present disclosure relates to methods and compositions for inducing an immune response in a subject comprising providing to the subject a peptide or protein vaccine and a binding agent, such as a bispecific antibody, binding to PD-L1 and CD137, such as human PD-L1 and human CD137, e.g., by co-administering to the subject a peptide or protein used for vaccination or a polynucleotide, in particular RNA, encoding a peptide or protein used for vaccination, and a binding agent binding to PD-L1 and CD137 or a polynucleotide, in particular RNA, encoding a binding agent binding to PD-L1 and CD137. The present disclosure further relates to medical preparations useful in the methods disclosed herein.

### BACKGROUND

Antigen-specific immunotherapy aims to enhance or induce specific immune responses in patients to control infectious or malignant diseases. The identification of a growing number of pathogen- and tumor-associated antigens (TAA) led to a broad collection of suitable targets for immunotherapy. Cells presenting immunogenic peptides (epitopes) derived from these antigens can be specifically targeted by either active or passive immunization strategies. Active immunization tends to induce and expand antigen-specific T cells in the patient, which are able to specifically recognize and kill diseased cells. In contrast passive immunization may rely on the adoptive transfer of T cells, which were expanded and optional genetically engineered in vitro (adoptive T cell therapy).

The evolution of the immune system resulted in vertebrates in a highly effective network based on two types of defense: the innate and the adoptive immunity. In contrast to the evolutionary ancient innate immune system that relies on invariant receptors recognizing common molecular patterns associated with pathogens, the adoptive immunity is based on highly specific antigen receptors on B cells (B lymphocytes) and T cells (T lymphocytes) and clonal selection. The immune system plays a crucial role during cancer development, progression and therapy. CD8+ T cells and NK cells can directly lyse tumor cells and high tumor-infiltration of these cells is generally regarded as favorable for the outcome of various tumor diseases. CD4+ T cells contribute to the anti-tumor immune response by secretion of IFNy or licensing of antigen-presenting dendritic cells (DCs), which in turn prime and activate CD8+ T cells (Kreiter S. et al.

Nature 520, 692-6 (2015)). The recognition and elimination of tumor cells by CD8+ T cells depends on antigen presentation via the Major Histocompatibility Complex (MHC) class I. CD8+as well as CD4+ tumor specific T-cell responses can be induced via vaccination. In the context of an mRNA based vaccine platform, mRNA may be delivered via liposomal formulation (RNA-LPX) into antigen presenting cells located in secondary lymphoid organs without requirement for any additional adjuvant (Kreiter, S. et al. Nature 520, 692-696 (2015); Kranz, L. M. et al. Nature 534, 396-401 (2016)).

Vaccines aim to induce endogenous disease-specific immune responses by active immunization. Different antigen formats can be used for vaccination including proteins, peptides or immunizing vectors such as RNA, DNA or viral vectors that can be applied either directly in vivo or in vitro by pulsing of dendritic cells (DCs) following transfer into the patient.

Vaccines for stimulating the immune system against an antigen expressed by diseased cells such as tumor cells show promising results, however, their effectiveness remains limited.

Programmed death ligand 1 (PD-L1, PDL1, CD274, B7H1) is a 33 kDa, single-pass type I membrane protein. Three isoforms of PD-L1 have been described, based on alternative splicing. PD-L1 belongs to the immunoglobulin (Ig) superfamily and contains one Ig-like C2-type domain and one Ig-like V-type domain. Freshly isolated T and B cells express negligible amounts of PD-L1 and a fraction (about 16%) of CD14+ monocytes constitutively express PD-L1. However, interferon-y (I FNy) is known to upregulate PD-L1 on tumor cells. PD-L1 obstructs anti-tumor immunity by 1) tolerizing tumor-reactive T cells by binding to its receptor programmed cell death protein 1 (PD-1) (CD279) on activated T cells; 2) rendering tumor cells resistant to CD8+ T cell and Fas ligand-mediated lysis by PD-1 signaling through tumor cell-expressed PD-L1; 3) tolerizing T cells by reverse signaling through T cell-expressed CD80 (B7.1); and 4) promoting the development and maintenance of induced T regulatory cells. PD-L1 is expressed in many human cancers, including melanoma, ovarian, lung and colon cancer (Latchman et al., 2004 Proc Natl Acad Sci USA 101, 10691-6).

CD137 (4-1BB, TNFRSF9) is a member of the tumor necrosis factor (TNF) receptor (TNFR) family. CD137 is a co-stimulatory molecule on CD8+ and CD4+ T cells, regulatory T cells (Tregs), natural killer (NK) and

NKT cells, B cells and neutrophils. On T cells, CD137 is not constitutively expressed, but induced upon T-cell receptor (TCR) activation. Stimulation via its natural ligand 4-1BBL or agonist antibodies leads to signaling using TNFR-associated factor (TRAF)-2 and TRAF-1 as adaptors. Early signaling by CD137 involves K-63 poly-ubiquitination reactions that ultimately result in activation of the nuclear factor (NF)-KB and mitogen-activated protein (MAP)-kinase pathways. Signaling leads to increased T cell co-stimulation, proliferation, cytokine production, maturation and prolonged CD8+ T-cell survival. Agonistic antibodies against CD137 have been shown to promote anti-tumor control by T cells in various pre-clinical models (Murillo et al. 2008 Clin. Cancer Res. 14(21): 6895-6906). Antibodies stimulating CD137 can induce survival and proliferation of T cells, thereby enhancing the anti-tumor immune response. Antibodies stimulating CD137 have been disclosed in the prior art, and include urelumab, a human IgG4 antibody (W02005035584) and utomilumab, a human IgG2 antibody (Fisher et al. 2012 Cancer Immunol. Immunother. 61: 1721-1733).

There is a need for novel strategies to increase the effectiveness of vaccines, in particular cancer vaccines.

It is demonstrated herein that multispecific antibodies that can bind both PD-L1 and CD137 are able to amplify vaccine-induced antigen-specific T cell responses and boost anti-tumoral immunity. Vaccination can be achieved by administering vaccine RNA, i.e., RNA encoding an antigen or epitope against which an immune response is to be induced.

### SUMMARY

The inventors surprisingly found that the effectiveness of vaccination (e.g., by administering RNA encoding peptides or proteins used for vaccination (RNA encoding antigen)) can be increased by co-administering multispecific (e.g., bispecific, trispecific etc.) binding agents binding to at least PD-L1 and CD137. In humans, CD137 is expressed on activated T cells, such as CD8+ T cells and CD4+ T cells, whereas PD-L1 is predominantly expressed on antigen-presenting cells (APCs) such as dendritic cells or tumor cells. In one embodiment, through its PD-L1 binding region, the binding agent binds PD-L1-expressing tumor cells or antigen-presenting cells (APCs), while through its CD137-binding region, the binding agent binds and activates T cells, resulting in conditional activation of the T cells. Without being bound by theory, the binding agent according to the invention may mediate clustering of CD137 when the binding agent simultaneously binds to PD-L1 and CD137. Clustering of CD137 by the binding agent is needed for sufficient activation of this receptor and CD137-mediated co-stimulation of T cells. In one embodiment, binding agents, such as bispecific antibodies, according to the invention may mediate cell-to-cell interaction between APCs and T cells by simultaneous binding of PD-L1 and CD137 on the cells.

Thus, this may lead to proliferation of antigen-specific T cells. In one embodiment, the binding agent brings T cells in close proximity to tumor cells, thereby facilitating tumor cell killing by T cells. In one embodiment, bringing PD-L1-expressing tumor cells and effector T cells, such as CD8+ T cells in close proximity to each other might initiate the release of interferon-y which in turn could upregulate PD-L1 on tumor cells, thus facilitating recruitment of more binding agent to the tumor and further enhance its killing. Thus, this may lead to further activation of T cells in the presence of tumor cells by binding of CD137 on the T cell, while binding of PD-L1 on tumor cells brings the T cell and tumor cell into close proximity. Thus, activation of T cells in the presence of tumor cells may lead to enhanced killing of tumor cells by the T cells. Further, the ability of the PD-L1 antigen-binding region, of the binding agent according to the invention, to inhibit binding of PD-L1 on tumor cells with PD-1 on T cells prevents that the tumor cell is able to induce T cell inhibition, and thereby escaping the anti-tumor effect of the activated T cell. PD-L1 binding to PD1 expressed on activated T cells may result in T cell inhibition. In one embodiment, the binding agent inhibits the binding of human PD-L1 to human PD-1 thus preventing PD-L1 from obstructing anti-tumor immunity through PD-1. Thus, the binding agent prevents that the T cells receive an inhibitory signal through PD-1/PD-L1 interaction, while receiving an activation signal through binding to the CD137 molecule resulting in signaling that strengthens T cell proliferation, activation, effector and memory functions. Binding agents such as bispecific antibodies may block the PD1-(PD-L1) inhibitory signaling and at the same time co-stimulate T cells via trans binding to the CD137 molecule expressed on activated T cells, with the activation occurring through the trans binding. The binding agents binding to at least PD-L1 and CD137 described herein may have an inert Fc region or alternatively no Fc binding region, and thereby do not induce complement-dependent cytotoxicity (CDC) or other Fc-mediated effector functions on the T cells when binding to CD137. The binding agents binding to at least PD-L1 and CD137 described herein may activate T cells. The binding agents binding to at least PD-L1 and CD137 described herein may be suitable for activating tumor-specific T cells such as tumor infiltrating T cells.

In one aspect, the invention relates to a method for treating a subject comprising administering to the subject:

a. a peptide or protein comprising an epitope for inducing an immune response against an antigen in the subject or a polynucleotide encoding the peptide or protein; and

b. a binding agent comprising a first antigen-binding region binding to PD-L1 and a second antigen-binding region binding to CD137, or a polynucleotide encoding the binding agent.

In one embodiment, the subject is a human. In one embodiment, the PD-L1 is human PD-L1 and/or the CD137 is human CD137. In one embodiment, said first antigen-binding region binding to PD-L1 inhibits the binding of PD-L1 to PD-1, such as the binding of human PD-L1 to human PD-1.

In one embodiment, said first antigen-binding region binding to PD-L1 comprises a heavy chain variable region (VH) comprising a HCDR3 having the sequence as set forth in SEQ ID NO: 13. In one embodiment, said first antigen-binding region binding to PD-L1 comprises a heavy chain variable region (VH) comprising a HCDR2 having the sequence as set forth in SEQ ID NO: 12. In one embodiment, said first antigen-binding region binding to PD-L1 comprises a heavy chain variable region (VH) comprising a HCDR1 having the sequence as set forth in SEQ ID NO: 11. In one embodiment, said first antigen-binding region binding to PD-L1 comprises a heavy chain variable region (VH) comprising a HCDR1, HCDR2, and HCDR3 sequence, wherein the HCDR1, HCDR2 and HCDR3 sequence comprises the sequence as set forth in SEQ ID NO: 11, 12, and 13, respectively. In one embodiment, said first antigen-binding region binding to PD-L1 comprises a light chain variable region (VL) comprising a LCDR3 having the sequence as set forth in SEQ ID NO: 16. In one embodiment, said first antigen-binding region binding to PD-L1 comprises a light chain variable region (VL) comprising a LCDR2 having the sequence DDN. In one embodiment, said first antigen-binding region binding to PD-L1 comprises a light chain variable region (VL) comprising a LCDR1 having the sequence as set forth in SEQ ID NO: 15. In one embodiment, said first antigen-binding region binding to PD-L1 comprises a light chain variable region (VL) comprising a LCDR1, LCDR2, and LCDR3 sequence, wherein the LCDR1, LCDR2 and LCDR3 sequence comprises the sequence as set forth in SEQ ID NO: 15, DDN, and 16, respectively. In one embodiment, said first antigen-binding region binding to PD-L1 comprises a heavy chain variable region (VH) comprising a HCDR3 sequence and a light chain variable region (VL) comprising a LCDR3 sequence, wherein the HCDR3 sequence comprises the sequence as set forth in SEQ ID NO: 13, and the LCDR3 sequence comprises the sequence as set forth in SEQ ID NO: 16. In one embodiment, said first antigen-binding region binding to PD-L1 comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises the sequence as set forth in SEQ ID NO: 11, 12, and 13, respectively, and the LCDR1, LCDR2 and LCDR3 sequence comprises the sequence as set forth in SEQ ID NO: 15, DDN, and 16, respectively. In one embodiment, said first antigen-binding region binding to PD-L1 comprises a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 10. In one embodiment, said first antigen-binding region binding to PD-L1 comprises a heavy chain variable region (VH), wherein the VH comprises the sequence as set forth in SEQ ID NO: 10. In one embodiment, said first antigen-binding region binding to PD-L1 comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 14. In one embodiment, said first antigen-binding region binding to PD-L1 comprises a light chain variable region (VL), wherein the VL comprises the sequence as set forth in SEQ ID NO: 14. In one embodiment, said first antigen-binding region binding to PD-L1 comprises a heavy chain variable region (VH) and a light chain variable region (VL), wherein the VH comprises a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 10 and the VL comprises a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 14. In one embodiment, said first antigen-binding region binding to PD-L1 comprises a heavy chain variable region (VH) and a light chain variable region (VL), wherein the VH comprises a sequence having at least at least 80% identity to the amino acid sequence of the VH sequence as set forth in SEQ ID NO: 10 and the VL comprises a sequence having at least 80% identity to the amino acid sequence of the VL sequence as set forth in SEQ ID NO: 14. In one embodiment, said first antigen-binding region binding to PD-L1 comprises a heavy chain variable region (VH) and a light chain variable region (VL), wherein the VH comprises a sequence having at least at least 90% identity to the amino acid sequence of the VH sequence as set forth in SEQ ID NO: 10 and the VL comprises a sequence having at least 90% identity to the amino acid sequence of the VL sequence as set forth in SEQ ID NO: 14. In one embodiment, said first antigen-binding region binding to PD-L1 comprises a heavy chain variable region (VH) and a light chain variable region (VL), wherein the VH comprises a sequence having at least at least 95% identity to the amino acid sequence of the VH sequence as set forth in SEQ ID NO: 10 and the VL comprises a sequence having at least 95% identity to the amino acid sequence of the VL sequence as set forth in SEQ ID NO: 14. In one embodiment, said first antigen-binding region binding to PD-L1 comprises a heavy chain variable region (VH) and a light chain variable region (VL), wherein the VH comprises the sequence as set forth in SEQ ID NO: 10 and the VL comprises the sequence as set forth in SEQ ID NO: 14. In one embodiment, said first antigen-binding region binding to PD-L1 comprises heavy and light chain variable regions of an antibody which competes for PD-L1 binding with and/or has the specificity for PD-L1 of an antibody comprising a heavy chain variable region (VH) or a light chain variable region (VL), or a combination thereof as set forth above.

In one embodiment, said second antigen-binding region binding to CD137 comprises a heavy chain variable region (VH) comprising a HCDR3 having the sequence as set forth in SEQ ID NO: 4. In one embodiment, said second antigen-binding region binding to CD137 comprises a heavy chain variable region (VH) comprising a HCDR2 having the sequence as set forth in SEQ ID NO: 3. In one embodiment, said second antigen-binding region binding to CD137 comprises a heavy chain variable region (VH) comprising a HCDR1 having the sequence as set forth in SEQ ID NO: 2. In one embodiment, said second antigen-binding region binding to CD137 comprises a heavy chain variable region (VH) comprising a HCDR1, HCDR2, and HCDR3 sequence, wherein the HCDR1, HCDR2 and HCDR3 sequence comprises the sequence as set forth in SEQ ID NO: 2, 3, and 4, respectively. In one embodiment, said second antigen-binding region binding to CD137 comprises a light chain variable region (VL) comprising a LCDR3 having the sequence as set forth in SEQ ID NO: 7. In one embodiment, said second antigen-binding region binding to CD137 comprises a light chain variable region (VL) comprising a LCDR2 having the sequence GAS. In one embodiment, said second antigen-binding region binding to CD137 comprises a light chain variable region (VL) comprising a LCDR1 having the sequence as set forth in SEQ ID NO: 6.

In one embodiment, said second antigen-binding region binding to CD137 comprises a light chain variable region (VL) comprising a LCDR1, LCDR2, and LCDR3 sequence, wherein the LCDR1, LCDR2 and LCDR3 sequence comprises the sequence as set forth in SEQ ID NO: 6, GAS, and 7, respectively. In one embodiment, said second antigen-binding region binding to CD137 comprises a heavy chain variable region (VH) comprising a HCDR3 sequence and a light chain variable region (VL) comprising a LCDR3 sequence, wherein the HCDR3 sequence comprises the sequence as set forth in SEQ ID NO: 4, and the LCDR3 sequence comprises the sequence as set forth in SEQ ID NO: 7. In one embodiment, said second antigen-binding region binding to CD137 comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises the sequence as set forth in SEQ ID NO: 2, 3, and 4, respectively, and the LCDR1, LCDR2 and LCDR3 sequence comprises the sequence as set forth in SEQ ID NO: 6, GAS, and 7, respectively. In one embodiment, said second antigen-binding region binding to CD137 comprises a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 1 or 8. In one embodiment, said second antigen-binding region binding to CD137 comprises a heavy chain variable region (VH), wherein the VH comprises the sequence as set forth in SEQ ID NO: 1 or 8. In one embodiment, said second antigen-binding region binding to CD137 comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 5 or 9. In one embodiment, said second antigen-binding region binding to CD137 comprises a light chain variable region (VL), wherein the VL comprises the sequence as set forth in SEQ ID NO: 5 or 9. In one embodiment, said second antigen-binding region binding to CD137 comprises a heavy chain variable region (VH) and a light chain variable region (VL), wherein the VH comprises a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 1 and the VL comprises a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 5. In one embodiment, said second antigen-binding region binding to CD137 comprises a heavy chain variable region (VH) and a light chain variable region (VL), wherein the VH comprises a sequence having at least at least 80% identity to the amino acid sequence of the VH sequence as set forth in SEQ ID NO: 1 and the VL comprises a sequence having at least 80% identity to the amino acid sequence of the VL sequence as set forth in SEQ ID NO: 5. In one embodiment, said second antigen-binding region binding to CD137 comprises a heavy chain variable region (VH) and a light chain variable region (VL), wherein the VH comprises a sequence having at least at least 90% identity to the amino acid sequence of the VH sequence as set forth in SEQ ID NO: 1 and the VL comprises a sequence having at least 90% identity to the amino acid sequence of the VL sequence as set forth in SEQ ID NO: 5. In one embodiment, said second antigen-binding region binding to CD137 comprises a heavy chain variable region (VH) and a light chain variable region (VL), wherein the VH comprises a sequence having at least at least 95% identity to the amino acid sequence of the VH sequence as set forth in SEQ ID NO: 1 and the VL comprises a sequence having at least 95% identity to the amino acid sequence of the VL sequence as set forth in SEQ ID NO: 5. In one embodiment, said second antigen-binding region binding to CD137 comprises a heavy chain variable region (VH) and a light chain variable region (VL), wherein the VH comprises the sequence as set forth in SEQ ID NO: 1 and the VL comprises the sequence as set forth in SEQ ID NO: 5. In one embodiment, said second antigen-binding region binding to CD137 comprises a heavy chain variable region (VH) and a light chain variable region (VL), wherein the VH comprises a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 8 and the VL comprises a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 9. In one embodiment, said second antigen-binding region binding to CD137 comprises a heavy chain variable region (VH) and a light chain variable region (VL), wherein the VH comprises a sequence having at least at least 80% identity to the amino acid sequence of the VH sequence as set forth in SEQ ID NO: 8 and the VL comprises a sequence having at least 80% identity to the amino acid sequence of the VL sequence as set forth in SEQ ID NO: 9. In one embodiment, said second antigen-binding region binding to CD137 comprises a heavy chain variable region (VH) and a light chain variable region (VL), wherein the VH comprises a sequence having at least at least 90% identity to the amino acid sequence of the VH sequence as set forth in SEQ ID NO: 8 and the VL comprises a sequence having at least 90% identity to the amino acid sequence of the VL sequence as set forth in SEQ ID NO: 9. In one embodiment, said second antigen-binding region binding to CD137 comprises a heavy chain variable region (VH) and a light chain variable region (VL), wherein the VH comprises a sequence having at least at least 95% identity to the amino acid sequence of the VH sequence as set forth in SEQ ID NO: 8 and the VL comprises a sequence having at least 95% identity to the amino acid sequence of the VL sequence as set forth in SEQ ID NO: 9. In one embodiment, said second antigen-binding region binding to CD137 comprises a heavy chain variable region (VH) and a light chain variable region (VL), wherein the VH comprises the sequence as set forth in SEQ ID NO: 8 and the VL comprises the sequence as set forth in SEQ ID NO: 9. In one embodiment, said second antigen-binding region binding to CD137 comprises heavy and light chain variable regions of an antibody which competes for CD137 binding with and/or has the specificity for CD137 of an antibody comprising a heavy chain variable region or a light chain variable region, or a combination thereof as set forth above.

In one embodiment, said second antigen-binding region binding to CD137 comprises a heavy chain variable region (VH) comprising a HCDR3 having the sequence as set forth in SEQ ID NO: 27. In one embodiment, said second antigen-binding region binding to CD137 comprises a heavy chain variable region (VH) comprising a HCDR2 having the sequence as set forth in SEQ ID NO: 26. In one embodiment, said second antigen-binding region binding to CD137 comprises a heavy chain variable region (VH) comprising a HCDR1 having the sequence as set forth in SEQ ID NO: 25. In one embodiment, said second antigen-binding region binding to CD137 comprises a heavy chain variable region (VH) comprising a HCDR1, HCDR2, and HCDR3 sequence, wherein the HCDR1, HCDR2 and HCDR3 sequence comprises the sequence as set forth in SEQ ID NO: 25, 26, and 27, respectively. In one embodiment, said second antigen-binding region binding to CD137 comprises a light chain variable region (VL) comprising a LCDR3 having the sequence as set forth in SEQ ID NO: 30. In one embodiment, said second antigen-binding region binding to CD137 comprises a light chain variable region (VL) comprising a LCDR2 having the sequence SAS. In one embodiment, said second antigen-binding region binding to CD137 comprises a light chain variable region (VL) comprising a LCDR1 having the sequence as set forth in SEQ ID NO: 29. In one embodiment, said second antigen-binding region binding to CD137 comprises a light chain variable region (VL) comprising a LCDR1, LCDR2, and LCDR3 sequence, wherein the LCDR1, LCDR2 and LCDR3 sequence comprises the sequence as set forth in SEQ ID NO: 29, SAS, and 30, respectively. In one embodiment, said second antigen-binding region binding to CD137 comprises a heavy chain variable region (VH) comprising a HCDR3 sequence and a light chain variable region (VL) comprising a LCDR3 sequence, wherein the HCDR3 sequence comprises the sequence as set forth in SEQ ID NO: 27, and the LCDR3 sequence comprises the sequence as set forth in SEQ ID NO: 30. In one embodiment, said second antigen-binding region binding to CD137 comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises the sequence as set forth in SEQ ID NO: 25, 26, and 27, respectively, and the LCDR1, LCDR2 and LCDR3 sequence comprises the sequence as set forth in SEQ ID NO: 29, SAS, and 30, respectively. In one embodiment, said second antigen-binding region binding to CD137 comprises a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 24. In one embodiment, said second antigen-binding region binding to CD137 comprises a heavy chain variable region (VH), wherein the VH comprises the sequence as set forth in SEQ ID NO: 24. In one embodiment, said second antigen-binding region binding to CD137 comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 28. In one embodiment, said second antigen-binding region binding to CD137 comprises a light chain variable region (VL), wherein the VL comprises the sequence as set forth in SEQ ID NO: 28. In one embodiment, said second antigen-binding region binding to CD137 comprises a heavy chain variable region (VH) and a light chain variable region (VL), wherein the VH comprises a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 24 and the VL comprises a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 28. In one embodiment, said second antigen-binding region binding to CD137 comprises a heavy chain variable region (VH) and a light chain variable region (VL), wherein the VH comprises a sequence having at least at least 80% identity to the amino acid sequence of the VH sequence as set forth in SEQ ID NO: 24 and the VL comprises a sequence having at least 80% identity to the amino acid sequence of the VL sequence as set forth in SEQ ID NO: 28. In one embodiment, said second antigen-binding region binding to CD137 comprises a heavy chain variable region (VH) and a light chain variable region (VL), wherein the VH comprises a sequence having at least at least 90% identity to the amino acid sequence of the VH sequence as set forth in SEQ ID NO: 24 and the VL comprises a sequence having at least 90% identity to the amino acid sequence of the VL sequence as set forth in SEQ ID NO: 28. In one embodiment, said second antigen-binding region binding to CD137 comprises a heavy chain variable region (VH) and a light chain variable region (VL), wherein the VH comprises a sequence having at least at least 95% identity to the amino acid sequence of the VH sequence as set forth in SEQ ID NO: 24 and the VL comprises a sequence having at least 95% identity to the amino acid sequence of the VL sequence as set forth in SEQ ID NO: 28. In one embodiment, said second antigen-binding region binding to CD137 comprises a heavy chain variable region (VH) and a light chain variable region (VL), wherein the VH comprises the sequence as set forth in SEQ ID NO: 24 and the VL comprises the sequence as set forth in SEQ ID NO: 28. In one embodiment, said second antigen-binding region binding to CD137 comprises heavy and light chain variable regions of an antibody which competes for CD137 binding with and/or has the specificity for CD137 of an antibody comprising a heavy chain variable region or a light chain variable region, or a combination thereof as set forth above.

In one embodiment, the binding agent comprises a first antigen-binding region binding to PD-L1 and a second antigen-binding region binding to CD137, wherein

a) the first antigen-binding region comprises a heavy chain variable region (VH) comprising a HCDR3 sequence, as set forth in SEQ ID NO: 13, and

b) the second antigen-binding region comprises a heavy chain variable region (VH) comprising a HCDR3 sequence, as set forth in SEQ ID NO: 4.

In one embodiment, the binding agent comprises a first antigen-binding region binding to PD-L1 and a second antigen-binding region binding to CD137, wherein

a) the first antigen-binding region comprises a light chain variable region (VL) comprising a LCDR3 sequence, as set forth in SEQ ID NO: 16, and

b) the second antigen-binding region comprises a light chain variable region (VL) comprising a LCDR3 sequence, as set forth in SEQ ID NO: 7.

In one embodiment, the binding agent comprises a first antigen-binding region binding to PD-L1 and a second antigen-binding region binding to CD137, wherein
a) the first antigen-binding region comprises a heavy chain variable region (VH) comprising a HCDR3 sequence, as set forth in SEQ ID NO: 13, and a light chain variable region (VL) comprising a LCDR3 sequence, as set forth in SEQ ID NO: 16, and
b) the second antigen-binding region comprises a heavy chain variable region (VH) comprising a HCDR3 sequence, as set forth in SEQ ID NO: 4, and a light chain variable region (VL) comprising a LCDR3 sequence, as set forth in SEQ ID NO: 7.

In one embodiment, the binding agent comprises a first antigen-binding region binding to PD-L1 and a second antigen-binding region binding to CD137, wherein

a) the first antigen-binding region comprises a heavy chain variable region (VH) comprising a HCDR1, HCDR2, and HCDR3 sequence, as set forth in SEQ ID NO: 11, 12, and 13, respectively, and a light chain variable region (VL) comprising a LCDR1, LCDR2, and LCDR3 sequence, as set forth in SEQ ID NO: 15, DDN, and 16, respectively, and

b) the second antigen-binding region comprises a heavy chain variable region (VH) comprising a HCDR1, HCDR2, and HCDR3 sequence, as set forth in SEQ ID NO: 2, 3, and 4, respectively, and a light chain variable region (VL) comprising a LCDR1, LCDR2, and LCDR3 sequence, as set forth in SEQ ID NO: 66L , GAS, and 7, respectively.

In one embodiment, the binding agent comprises a first antigen-binding region binding to PD-L1 and a second antigen-binding region binding to CD137, wherein

a) the first antigen-binding region comprises a heavy chain variable region (VH) and a light chain variable region (VL), wherein the VH comprises a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 10 and the VL comprises a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 14, and

b) the second antigen-binding region comprises a heavy chain variable region (VH) and a light chain variable region (VL), wherein the VH comprises a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 8 and the VL comprises a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 9. In one embodiment, the binding agent comprises a first antigen-binding region binding to PD-L1 and a second antigen-binding region binding to CD137, wherein

a) the first antigen-binding region comprises a heavy chain variable region (VH) and a light chain variable region (VL), wherein the VH comprises a sequence having at least 80% identity to the amino acid sequence of the VH sequence as set forth in SEQ ID NO: 10 and the VL comprises a sequence having at least 80% identity to the amino acid sequence of the VL sequence as set forth in SEQ ID NO: 14, and

b) the second antigen-binding region comprises a heavy chain variable region (VH) and a light chain variable region (VL), wherein the VH comprises a sequence having at least 80% identity to the amino acid sequence of the VH sequence as set forth in SEQ ID NO: 8 and the VL comprises a sequence having at least 80% identity to the amino acid sequence of the VL sequence as set forth in SEQ ID NO: 9. In one embodiment, the binding agent comprises a first antigen-binding region binding to PD-L1 and a second antigen-binding region binding to CD137, wherein

a) the first antigen-binding region comprises a heavy chain variable region (VH) and a light chain variable region (VL), wherein the VH comprises a sequence having at least 90% identity to the amino acid sequence of the VH sequence as set forth in SEQ ID NO: 10 and the VL comprises a sequence having at least 90% identity to the amino acid sequence of the VL sequence as set forth in SEQ ID NO: 14, and

b) the second antigen-binding region comprises a heavy chain variable region (VH) and a light chain variable region (VL), wherein the VH comprises a sequence having at least 90% identity to the amino acid sequence of the VH sequence as set forth in SEQ ID NO: 8 and the VL comprises a sequence having at least 90% identity to the amino acid sequence of the VL sequence as set forth in SEQ ID NO: 9. In one embodiment, the binding agent comprises a first antigen-binding region binding to PD-L1 and a second antigen-binding region binding to CD137, wherein

a) the first antigen-binding region comprises a heavy chain variable region (VH) and a light chain variable region (VL), wherein the VH comprises a sequence having at least 95% identity to the amino acid sequence of the VH sequence as set forth in SEQ ID NO: 10 and the VL comprises a sequence having at least 95% identity to the amino acid sequence of the VL sequence as set forth in SEQ ID NO: 14, and

b) the second antigen-binding region comprises a heavy chain variable region (VH) and a light chain variable region (VL), wherein the VH comprises a sequence having at least 95% identity to the amino acid sequence of the VH sequence as set forth in SEQ ID NO: 8 and the VL comprises a sequence having at least 95% identity to the amino acid sequence of the VL sequence as set forth in SEQ ID NO: 9. In one embodiment, the binding agent comprises a first antigen-binding region binding to PD-L1 and a second antigen-binding region binding to CD137, wherein

a) the first antigen-binding region comprises a heavy chain variable region (VH) and a light chain variable region (VL), wherein the VH comprises the amino acid sequence of the VH sequence as set forth in SEQ ID NO: 10 and the VL comprises the amino acid sequence of the VL sequence as set forth in SEQ ID NO: 14, and

b) the second antigen-binding region comprises a heavy chain variable region (VH) and a light chain variable region (VL), wherein the VH comprises the amino acid sequence of the VH sequence as set forth in SEQ ID NO: 8 and the VL comprises the amino acid sequence of the VL sequence as set forth in SEQ ID NO: 9.

In one embodiment, said first antigen-binding region binding to PD-L1 comprises heavy and light chain variable regions of an antibody which competes for PD-L1 binding with and/or has the specificity for PD-L1 of an antibody comprising a heavy chain variable region or a light chain variable region, or a combination thereof of a first antigen-binding region binding to PD-L1 as set forth above and said second antigen-binding region binding to CD137 comprises heavy and light chain variable regions of an antibody which competes for CD137 binding with and/or has the specificity for CD137 of an antibody comprising a heavy chain variable region or a light chain variable region, or a combination thereof of a second antigen-binding region binding to CD137 as set forth above.

In one embodiment, the binding agent comprises a first antigen-binding region binding to PD-L1, which is derived from a human antibody, and/or a second antigen-binding region binding to CD137, which is derived from a humanized antibody.

In one embodiment, the binding agent is in the format of a full-length antibody. In one embodiment, the binding agent is in the format of an antibody fragment. In one embodiment, the binding agent is a multispecific antibody such as a bispecific antibody.

In one embodiment of the invention, the binding agent, comprises a polypeptide wherein the polypeptide is a heavy chain (HC). In one embodiment of the invention the heavy chain (HC) comprises a heavy chain variable region (VH) and a heavy chain constant region (CH). In one embodiment of the invention, the heavy chain constant region (CH) comprises a constant region domain 1 region (CH1), a hinge region, a constant region domain 2 region (CH2), and a constant region domain 3 region (CH3).

In one embodiment of the invention, the binding agent, in particular in the form of a multispecific antibody such as a bispecific antibody, comprises (i) a polypeptide comprising a first heavy chain variable region

(VH) and further comprising a first heavy chain constant region (CH) and (ii) a polypeptide comprising a second heavy chain variable region (VH) and further comprising a second heavy chain constant region (CH). Alternatively or additionally, in one embodiment of the invention, the binding agent, in particular in the form of a multispecific antibody such as a bispecific antibody, comprises (i) a polypeptide comprising a first light chain variable region (VL) and further comprising a first light chain constant region (CL) and (ii) a polypeptide comprising a second light chain variable region (VL) and further comprising a second light chain constant region (CL).

In one embodiment of the invention, the binding agent is an antibody, such as a multispecific, preferably bispecific antibody, comprising a first binding arm and a second binding arm, wherein

a. the first binding arm comprises i) a polypeptide comprising a first heavy chain variable region (VH) and a first heavy chain constant region (CH) and ii) a polypeptide comprising a first light chain variable region (VL) and a first light chain constant region (CL) and;

b. the second binding arm comprises iii) a polypeptide comprising a second heavy chain variable region (VH) and a second heavy chain constant region (CH) and iv) a polypeptide comprising a second light chain variable region (VL) and a second light chain constant region (CL). In one embodiment, said first heavy chain variable region (VH) is comprised by a first antigen-binding region binding to PD-L1 and said second heavy chain variable region (VH) is comprised by a second antigen-binding region binding to CD137 and/or said first light chain variable region (VL) is comprised by a first antigen-binding region binding to PD-L1 and said second light chain variable region (VL) is comprised by a second antigen-binding region binding to CD137.

In one embodiment, said first heavy chain constant region (CH) comprises the amino acid sequence as set forth in SEQ ID NO: 18 and/or said second heavy chain constant region (CH) comprises the amino acid sequence as set forth in SEQ ID NO: 17. In one embodiment, said first heavy chain constant region (CH) comprises the amino acid sequence as set forth in SEQ ID NO: 17 and/or said second heavy chain constant region (CH) comprises the amino acid sequence as set forth in SEQ ID NO: 18. In one embodiment, said first light chain constant region (CL) comprises the amino acid sequence as set forth in SEQ ID NO: 20 and/or said second light chain constant region (CL) comprises the amino acid sequence as set forth in SEQ ID NO: 19. In one embodiment, said first light chain constant region (CL) comprises the amino acid sequence as set forth in SEQ ID NO: 19 and/or said second light chain constant region (CL) comprises the amino acid sequence as set forth in SEQ ID NO: 20.

In one embodiment, said first heavy chain constant region (CH) comprises the amino acid sequence as set forth in SEQ ID NO: 18, said second heavy chain constant region (CH) comprises the amino acid sequence as set forth in SEQ ID NO: 17, said first light chain constant region (CL) comprises the amino acid sequence as set forth in SEQ ID NO: 20 and said second light chain constant region (CL) comprises the amino acid sequence as set forth in SEQ ID NO: 19.

In one embodiment of the invention, the binding agent, in particular in the form of a multispecific antibody such as a bispecific antibody, comprises a first and second heavy chain constant region (CH) comprising one or more of a constant region domain 1 region (CH1 region), a hinge region, a CH2 region and a CH3 region, preferably at least a hinge region, a CH2 region and a CH3 region.

In one embodiment of the invention, the binding agent, in particular in the form of a multispecific antibody such as a bispecific antibody, is of an isotype selected from the group consisting of IgG1, IgG2, IgG3, and IgG4. In one embodiment of the invention the isotype is selected from the group consisting of human IgG1, human IgG2, human IgG3 and human IgG4.

In one embodiment of the invention, the first binding arm is derived from a full-length antibody. In one embodiment of the invention, the first binding arm is derived from a monoclonal antibody. In one embodiment of the invention, the first binding arm is derived from a full-length IgG1, λ (lambda) or IgG1, κ (kappa) antibody.

In one embodiment of the invention, the second binding arm is derived from a full-length antibody. In one embodiment of the invention, the second binding arm is derived from a monoclonal antibody. In one embodiment of the invention, the second binding arm is derived from a full-length IgG1, A (lambda) or IgG1, κ (kappa) antibody.

In one embodiment of the invention, the first and second binding arms are derived from full-length antibodies, such as from full-length IgG1, λ (lambda) or IgG1, κ (kappa) antibodies. In one embodiment of the invention, the first and second binding arms are derived from monoclonal antibodies. In one embodiment of the invention, the first antigen binding region or binding arm is derived from an IgG1 lambda and the second antigen binding region or binding arm is derived from an IgG1 kappa. Antibodies described herein include IgG1, IgG2, IgG3 and IgG4 antibodies and combinations thereof, wherein the heavy chains are of different isotypes and/or subclasses. In various embodiments, the antibody is an IgG1 antibody, more particularly an IgG1, kappa or IgG1, lambda isotype (i.e. IgG1, κ, λ), an IgG2a antibody (e.g. IgG2a, κ, λ), an IgG2b antibody (e.g. IgG2b, κ, λ), an IgG3 antibody (e.g. IgG3, κ, λ) or an IgG4 antibody (e.g. IgG4, κ, λ).

In one embodiment of the invention, the binding agent is a multispecific such as a bispecific binding agent. In one embodiment of the invention, the binding agent is an antibody (in particular a multispecific antibody, e.g., a bispecific antibody), such as a chimeric or humanized or human antibody. In one embodiment of the invention, the binding agent is in the format of a full-length antibody or an antibody fragment. In one embodiment of the invention, the first antigen-binding region is derived from a monoclonal antibody. In one embodiment of the invention, the second antigen-binding region is derived from a monoclonal antibody. In one embodiment of the invention, the first antigen-binding region is derived from a monoclonal antibody and the second antigen-binding region is derived from a monoclonal antibody.

In one embodiment of the invention, the binding agent is a full-length IgG1 antibody. In one embodiment of the invention, the binding agent is a full-length human IgG1 antibody. In one embodiment of the invention, the binding agent is a full-length human IgG1 antibody with one or more mutations in the constant region.

In one embodiment of the invention, the binding agent is chimeric, humanized or human antibody. In embodiments of the invention wherein the binding agent is a bispecific antibody, both half-molecules can be human, humanized or chimeric, or the half-molecules can differ in character with respect to sequence origin.

In one embodiment of the invention, the binding agent, in particular in the form of a multispecific antibody such as a bispecific antibody, comprises a first and second heavy chain constant region (CH) comprising a CH3 region and wherein the two CH3 regions comprise asymmetrical mutations. In a preferred embodiment of the invention, the binding agent, in particular in the form of a multispecific antibody such as a bispecific antibody comprises a first and second heavy chain constant region (CH), wherein each of said first and second heavy chains comprises at least a hinge region, a CH2 and a CH3 region, wherein in said first heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and in said second heavy chain at least one of the amino acids in a position corresponding to a position selected from the group consisting of T366, L368, K370, D399, F405, Y407, and K409 in a human IgG1 heavy chain according to EU numbering has been substituted, and wherein said first and said second heavy chains are not substituted in the same positions.

Most preferably, (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

In one embodiment of the invention, the binding agent is an antibody, such as a multispecific, preferably bispecific antibody, wherein said antibody induces Fc-mediated effector function to a lesser extent compared to another antibody comprising the same first and second antigen binding regions and two heavy chain constant regions (CHs) comprising human IgG1 hinge, CH2 and CH3 regions. In one embodiment of the invention, said first and second heavy chain constant regions are modified so that the antibody induces Fc-mediated effector function to a lesser extent compared to an antibody which is identical except for comprising non-modified first and second heavy chains.

In one embodiment of the invention, said Fc-mediated effector function is measured by binding to IgG Fc (Fcy) receptors, binding to C1q, or induction of Fc-mediated cross-linking of FcRs. In a preferred embodiment of the invention, said Fc-mediated effector function is measured by binding to C1 q .

In one embodiment of the invention, said first and second heavy chain constant regions have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA. In one embodiment of the invention, in at least one of said first and second heavy chain constant regions one or more amino acids in the positions corresponding to positions L234, L235, D265, N297, and P331 in a human IgG1 heavy chain according to EU numbering, are not L, L, D, N, and P, respectively. In one embodiment of the invention, the positions corresponding to positions L234 and L235 in a human IgG1 heavy chain according to EU numbering are F and E, respectively, in said first and second heavy chain constant regions. In one embodiment of the invention, the positions corresponding to positions L234, L235, and D265 in a human IgG1 heavy chain according to EU numbering are F, E, and A, respectively, in said first and second heavy chains.

In a further particularly preferred embodiment, the binding agent is a PD-L1xCD137 bispecific antibody comprising a first and second heavy chain constant region, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain is L, and the position corresponding to K409 in a human IgG1 heavy chain according to EU numbering of the second heavy chain is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

In one embodiment of the invention, the binding agent induces and/or enhances proliferation of T cells. In one embodiment of the invention, said T cells are CD4+ and/or CD8+ T cells. In one embodiment of the invention, the binding agent activates CD137 signaling only when the second antigen-binding region binds to PD-L1.

In one embodiment of the invention, proliferation of T cells is measured by co-culturing T-cells expressing a specific T cell receptor (TCR) with dendritic cells (DCs) presenting the corresponding antigen on the major histocompatibility complex, which is recognized by the TCR.

In one embodiment, the peptide or protein comprising an epitope for inducing an immune response against an antigen in the subject or the polynucleotide encoding the peptide or protein and the binding agent or the polynucleotide encoding the binding agent are administered sequentially. In one embodiment, the binding agent or the polynucleotide encoding the binding agent is administered following administration of the peptide or protein comprising an epitope for inducing an immune response against an antigen in the subject or the polynucleotide encoding the peptide or protein. In one embodiment, the binding agent or the polynucleotide encoding the binding agent is administered 6 hours or later, 12 hours or later or 24 hours or later following administration of the peptide or protein comprising an epitope for inducing an immune response against an antigen in the subject or the polynucleotide encoding the peptide or protein. In one embodiment, the binding agent or the polynucleotide encoding the binding agent is administered between 12 hours and 48 hours following administration of the peptide or protein comprising an epitope for inducing an immune response against an antigen in the subject or the polynucleotide encoding the peptide or protein.

In one embodiment, the polynucleotide encoding the peptide or protein comprising an epitope for inducing an immune response against an antigen in the subject and/or the polynucleotide encoding the binding agent is RNA.

In one embodiment, the method of the invention comprises administering to the subject:

a. RNA encoding the peptide or protein comprising an epitope for inducing an immune response against an antigen in the subject; and b. the binding agent.

In one embodiment, administering the binding agent or the polynucleotide encoding the binding agent increases the number of CD8 positive T cells which are specific for the antigen or cells expressing the antigen.

In one embodiment, the method of the invention is a method for inducing an immune response in the subject. In one embodiment, the method of the invention is a method for inducing an immune response against the antigen or cells expressing the antigen in the subject. In one embodiment, the method of the invention is a method for treating or preventing cancer in the subject, wherein the antigen is a tumor-associated antigen.

In one aspect, the invention relates to a medical preparation, comprising:

a. a peptide or protein comprising an epitope for inducing an immune response against an antigen in a subject, or a polynucleotide encoding the peptide or protein; and

b. a binding agent comprising a first antigen-binding region binding to PD-L1 and a second antigen-binding region binding to CD137, or a polynucleotide encoding the binding agent.

In one embodiment, the binding agent is a binding agent as defined above for the method of the invention. In one embodiment, the first antigen-binding region binding to PD-L1 and/or the second antigen-binding region binding to CD137 is as defined above for the method of the invention.

In one embodiment, the medical preparation comprises:

a. RNA encoding the peptide or protein comprising an epitope for inducing an immune response against an antigen in a subject; and

b. the binding agent.

In one embodiment, the medical preparation is a kit. In one embodiment, the medical preparation comprises each component a. and b. in a separate container. In one embodiment, the medical preparation further comprises instructions for use of the medical preparation for treating or preventing cancer, wherein the antigen is a tumor-associated antigen.

In one embodiment, the medical preparation is for pharmaceutical use. In one embodiment, the pharmaceutical use comprises a therapeutic or prophylactic treatment of a disease or disorder. In one embodiment, the medical preparation is for use in a method for treating or preventing cancer in a subject, wherein the antigen is a tumor-associated antigen.

In one embodiment, the medical preparation is a pharmaceutical composition. In one embodiment, the pharmaceutical composition further comprises one or more pharmaceutically acceptable carriers, diluents and/or excipients.

In one embodiment of the medical preparation, the RNA is present in a form selected from a liquid form, a solid form, or a combination thereof. In one embodiment, the solid form is a frozen form or a dehydrated form. In one embodiment, the dehydrated form is a freeze-dried or spray-dried form.

In one aspect, the invention relates to an agent or composition described herein such as a peptide or protein comprising an epitope for inducing an immune response against an antigen in a subject, a polynucleotide encoding the peptide or protein, a binding agent comprising a first antigen-binding region binding to PD-L1 and a second antigen-binding region binding to CD137, a polynucleotide encoding the binding agent, or a medical preparation for use in a method described herein.

In one embodiment of all aspects described herein, RNA encoding a peptide or protein comprising an epitope is delivered to the lymphatic system such as secondary lymphoid organs for expression of the encoded protein and/or is formulated for delivery to the lymphatic system such as secondary lymphoid organs.

### BRIEF DESCRIPTION OF THE DRAWINGS

FIG. 1: Expansion of antigen-specific CD8* T cells upon treatment with mouse-reactive PD-L1x4-1BB bsAb compared to isotype control in combination with a TRP1 RNA vaccine in C57BL16 mice.

C57BL/6 mice (n=5 per group) were vaccinated intravenously (i.v.) with 20 pg TRP1 RNA-LPX at day 1 and subsequently treated intraperitoneally (i.p.) with different doses of PD-L1x4-1BB bsAb at days 2, and 5. The control group received the TRP1 vaccine together with isotype control antibody. (A) Frequency of TRP1-specific CD8+ T cells in all peripheral CD8+ T cells, absolute number per pL blood of (B) TRP1-specific CD8+ T cells, (C) total CD8+ T cells, and (D) CD45±lymphocytes determined seven days after vaccination via flow cytometry from C57BL/6 mice as described in Example 1. Dots represent individual mice, lines represent the group mean. Mean±SD is shown. Statistical significance was determined using one-way ANOVA followed by Dunnett's multiple comparisons test. All analyses were two-tailed and carried out using GraphPad Prism 6. ns P>0.05, *P<0.05, **P<0.01, ***P<0.001, ****P<0.0001.

FIG. 2: Expansion of antigen-specific CD8* T cells upon treatment with mouse-reactive PD-L1x4-1BB bsAb or anti-PD-L1 mAb compared to isotype control in combination with a TRP1 RNA vaccine in C57BL16 mice.

C57BL/6 mice (n=5 per group) were vaccinated i.v. with 20 pg TRP1 RNA-LPX at day 1 and subsequently treated i.p with an optimal dose of PD-L1x4-1BB bsAb (50 pg) or anti-PD-L1 mAb (250 pg) at day 2. The control group received the TRP1 vaccine together with isotype control antibody. (A) Frequency of TRP1-specific CD8+ T cells in all peripheral CD8+ T cells, absolute number per pL blood of (B) TRP1-specific CD8+ T cells, (C) total CD8+ T cells, and (D) CD45±lymphocytes determined seven days after vaccination via flow cytometry from C57BL/6 mice as described in Example 2. Dots represent individual mice, lines represent the group mean. Mean±SD is shown. Statistical significance was determined using one-way ANOVA followed by Dunnett's multiple comparisons test. All analyses were two-tailed and carried out using GraphPad Prism 6. ns P>0.05, *P<0.05, **P<0.01, ***P<0.001.

FIG. 3: Reduced tumor growth resulting in improved survival after treatment with mouse-reactive PD-L1x4-1BB bsAb compared to isotype control in combination with a therapeutic RNA vaccine in the murine melanoma model B16.

C57BL/6 mice (n=10 per group) were inoculated subcutaneously (s.c.) with 3×10⁵ B16-F10 melanoma cells and vaccinated intravenously (i.v.) three times weekly (day 8, 15, 22) with 20 μg TRP1 RNA-LPX with subsequent PD-L1x4-1BB bsAb treatment at days 9, 12, 16, 19, 23, and 26. Control groups received either an RNA vaccine not coding for any antigen (irr vaccine) together with PD-L1x4-1BB bsAb or isotype control antibody, or TRP1 vaccine together with isotype control antibody. (A) Tumor growth of individual mice and (B) survival of mice treated with PD-L1x4-1BB bsAb or isotype control antibody with or without TRP1 vaccine. Ratios in (A) represent the number of tumor-free mice over the total number of mice per group.

FIG. 4: Expansion of tumor antigen-specific CD8+ T cells upon treatment with mouse-reactive PD-L1x4-1BB bsAb compared to isotype control in combination with a therapeutic RNA vaccine in the murine melanoma model B16.

(A) Kinetics of TRP1-specific CD8+ T cell numbers per pL blood after two rounds of vaccination (day 8, 15) and four applications of antibody (days 9, 12, 16, and 19). (B) Absolute numbers of TRP1-specific CD8+ T cells and total CD8+ T cells at 7 days after 1st vaccination. (C) Absolute numbers of TRP1-specific CD8+ T cells and total CD8+ T cells at 7 days after 2^{nd} vaccination. (D) Total CD45+ lymphocyte count at 7 days after 1st and 2^{nd} vaccination. Cell numbers were determined via flow cytometry from C57BL/6 mice described in Example 3 and FIG. 3. Dots in (A) represent the group mean with error bars representing the SD. Dots in (B-D) represent individual mice, lines represent the group mean. Mean±SD is shown. Statistical significance was determined using one-way ANOVA followed by Dunnett's multiple comparisons test. All analyses were two-tailed and carried out using GraphPad Prism 6. ns P>0.05, *P <0 .05, **P <0 .01, ***P <0 .001, ****P <0 .0001.

### DETAILED DESCRIPTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. KaIbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “about” means approximately or nearly, and in the context of a numerical value or range set forth herein in one embodiment means ±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

In the following, definitions will be provided which apply to all aspects of the present disclosure. The following terms have the following meanings unless otherwise indicated. Any undefined terms have their art recognized meanings.

Terms such as “reduce”, “decrease”, “inhibit” or “impair” as used herein relate to an overall decrease or the ability to cause an overall decrease, preferably of 5% or greater, 10% or greater, 20% or greater, more preferably of 50% or greater, more preferably of 75% or greater and most preferably 100%, in the level, e.g. in the level of binding.

Terms such as “increase”, “enhance” or “exceed” preferably relate to an increase or enhancement by about at least 10%, preferably at least 20%, preferably at least 30%, more preferably at least 40%, more preferably at least 50%, even more preferably at least 80%, and most preferably at least 100%, at least 200%, at least 500%, or even more.

According to the disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” or “polypeptide” refers to large peptides, in particular peptides having at least about 151 amino acids, but the terms “peptide”, “protein” and “polypeptide” are used herein usually as synonyms.

A “therapeutic protein” has a positive or advantageous effect on a condition or disease state of a subject when provided to the subject in a therapeutically effective amount. In one embodiment, a therapeutic protein has curative or palliative properties and may be administered to ameliorate, relieve, alleviate, reverse, delay onset of or lessen the severity of one or more symptoms of a disease or disorder. A therapeutic protein may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Examples of therapeutically active proteins include, but are not limited to, peptides or proteins used for vaccination.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable e.g. by translation of a truncated open reading frame that lacks the 3′-end of the open reading frame. A fragment shortened at the N-terminus (C-terminal fragment) is obtainable e.g. by translation of a truncated open reading frame that lacks the 5′-end of the open reading frame, as long as the truncated open reading frame comprises a start codon that serves to initiate translation. A fragment of an amino acid sequence comprises e.g. at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

By “variant” or “variant protein” or “variant polypeptide” herein is meant a protein that differs from a wild type protein by virtue of at least one amino acid modification. The parent polypeptide may be a naturally occurring or wild type (WT) polypeptide, or may be a modified version of a wild type polypeptide. Preferably, the variant polypeptide has at least one amino acid modification compared to the parent polypeptide, e.g., from 1 to about 20 amino acid modifications, and preferably from 1 to about 10 or from 1 to about 5 amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild type polypeptide, or a variant or engineered version of a wild type polypeptide.

By “wild type” or “WT” or “native” herein is meant an amino acid sequence that is found in nature, including allelic variations. A wild type protein or polypeptide has an amino acid sequence that has not been intentionally modified.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all splice variants, posttranslationally modified variants, conformations, isoforms and species homologs, in particular those which are naturally expressed by cells. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains.

Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids.

In the context of the present invention, conservative substitutions may be defined by substitutions within the classes of amino acids reflected in the following table:

| | |
|---|---|
| Acidic Residues | Asp (D) and Glu (E) |
| Basic Residues | Lys (K), Arg (R), and His (H) |
| Hydrophilic Uncharged | Ser (S), Thr (T), Asn (N), and |
| Residues | Gln (Q) |
| Aliphatic Uncharged | Gly (G), Ala (A), Val (V), Leu (L), |
| Residues | and Ile (I) |
| Non-polar Uncharged | Cys (C), Met (M), and Pro (P) |
| Residues | |
| Aromatic Residues | Phe (F), Tyr (Y), and Trp (W) |

Preferably the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, preferably continuous amino acids. In preferred embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences.

The term “percentage identity” is intended to denote a percentage of amino acid residues which are identical between the two sequences to be compared, obtained after the best alignment, this percentage being purely statistical and the differences between the two sequences being distributed randomly and over their entire length. Sequence comparisons between two amino acid sequences are conventionally carried out by comparing these sequences after having aligned them optimally, said comparison being carried out by segment or by “window of comparison” in order to identify and compare local regions of sequence similarity. The optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The percentage identity is calculated by determining the number of identical positions between the two sequences being compared, dividing this number by the number of positions compared and multiplying the result obtained by 100 so as to obtain the percentage identity between these two sequences.

Homologous amino acid sequences exhibit according to the disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one embodiment, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to peptides or proteins used for vaccination, one particular function is to induce an immune response directed to and/or induced by the amino acid sequence from which the fragment or variant is derived, e.g., a naturally occurring antigen. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response to a target molecule. In one embodiment, the modifications in the amino acid sequence of the parent molecule or sequence do not significantly affect or alter the immunogenic characteristics of the molecule or sequence. In different embodiments, immunogenicity of the functional fragment or functional variant may be reduced but still significantly present, e.g., immunogenicity of the functional variant may be at least 50%, at least 60%, at least 70%, at least 80%, or at least 90% of the parent molecule or sequence. However, in other embodiments, immunogenicity of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the peptides or proteins suitable for vaccination herein may be altered such that they vary in sequence from the naturally occurring or native target sequences from which they were derived, while retaining the desirable activity to induce an immune response against the native sequences.

As used herein, an “instructional material” or “instructions” includes a publication, a recording, a diagram, or any other medium of expression which can be used to communicate the usefulness of the compositions and methods of the invention. The instructional material of the kit of the invention may, for example, be affixed to a container which contains the compositions of the invention or be shipped together with a container which contains the compositions. Alternatively, the instructional material may be shipped separately from the container with the intention that the instructional material and the compositions be used cooperatively by the recipient.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

The term “recombinant” in the context of the present invention means “made through genetic engineering”. Preferably, a “recombinant object” such as a recombinant cell in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

The term “genetic modification” includes the transfection of cells with nucleic acid. The term “transfection” relates to the introduction of nucleic acids, in particular RNA, into a cell. For purposes of the present invention, the term “transfection” also includes the introduction of a nucleic acid into a cell or the uptake of a nucleic acid by such cell, wherein the cell may be present in a subject, e.g., a patient. Thus, according to the present invention, a cell for transfection of a nucleic acid described herein can be present in vitro or in vivo, e.g. the cell can form part of an organ, a tissue and/or an organism of a patient. According to the invention, transfection can be transient or stable. For some applications of transfection, it is sufficient if the transfected genetic material is only transiently expressed. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection. Generally, nucleic acid encoding a peptide or protein used for vaccination is transiently transfected into cells. RNA can be transfected into cells to transiently express its coded protein.

The term “immune effector cell” or “effector cell” in the context of the present invention relates to a cell which exerts effector functions during an immune reaction. For example, immune effector cells comprise T cells (cytotoxic T cells, helper T cells, tumor infiltrating T cells), B cells, natural killer cells, neutrophils, macrophages, and dendritic cells. The terms “T cell” and “T lymphocyte” are used interchangeably herein and include T helper cells (CD4+ T cells) and cytotoxic T cells (CTLs, CD8+ T cells) which comprise cytolytic T cells. The term “MHC-dependent T cell” or similar terms relate to a T cell which recognizes an antigen when presented in the context of MHC and preferably exerts effector functions of T cells, e.g., killing of target cells expressing an antigen.

T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptor (TCR). The thymus is the principal organ responsible for the maturation of T cells. Several different subsets of T cells have been discovered, each with a distinct function.

T helper cells assist other white blood cells in immunologic processes, including maturation of B cells into plasma cells and activation of cytotoxic T cells and macrophages, among other functions. These cells are also known as CD4+ T cells because they express the CD4 glycoprotein on their surface. Helper T cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T cells destroy virally infected cells and tumor cells, and are also implicated in transplant rejection. These cells are also known as CD8+ T cells since they express the CD8 glycoprotein on their surface. These cells recognize their targets by binding to antigen associated with MHC class I, which is present on the surface of nearly every cell of the body.

All T cells have a T cell receptor (TCR) existing as a complex of several proteins. The TCR of a T cell is able to interact with immunogenic peptides (epitopes) bound to major histocompatibility complex (MHC) molecules and presented on the surface of target cells. Specific binding of the TCR triggers a signal cascade inside the T cell leading to proliferation and differentiation into a maturated effector T cell. In the majority of T cells, the actual T cell receptor is composed of two separate peptide chains, which are produced from the independent T cell receptor alpha and beta (TCRα and TCRβ) genes and are called α- and β-TCR chains. A much less common (2% of total T cells) group of T cells, the γδ T cells (gamma delta T cells) possess a distinct T cell receptor (TCR) on their surface, which is made up of one γ-chain and one δ-chain.

All T cells originate from hematopoietic stem cells in the bone marrow. Hematopoietic progenitors derived from hematopoietic stem cells populate the thymus and expand by cell division to generate a large population of immature thymocytes. The earliest thymocytes express neither CD4 nor CD8, and are therefore classed as double-negative (CD4− CD8−) cells. As they progress through their development they become double-positive thymocytes (CD4+ CD8+), and finally mature to single-positive (CD4+ CD8- or CD4− CD8+) thymocytes that are then released from the thymus to peripheral tissues.

As used herein, the term “NK cell” or “Natural Killer cell” refers to a subset of peripheral blood lymphocytes defined by the expression of CD56 or CD16 and the absence of the T cell receptor.

MHC molecules in humans are normally referred to as HLA (human leukocyte antigen) molecules. There are two principal classes of MHC molecules: class I and class II. MHC class I antigens are found on nearly all nucleated cells of the body. The primary function of this class of MHC molecules is to display (or present) peptide fragments of intracellular proteins to CTLs. Based on this display, CTLs will attack those displaying MHC-bound peptides, including disease-associated peptides (antigens) such as cancer antigens. CD8-positive T cells are usually cytotoxic (therefore named cytotoxic T cells=CTL), recognize peptides of 9 to 10 amino acids which are intracellularly processed from proteins of any subcellular localization and which are presented on the cellular surface by MHC class I molecules. Thus, the surface expression of MHC class I molecules plays a crucial role in determining the susceptibility of target cells to CTLs.

### Nucleic Acids

The term “polynucleotide” or “nucleic acid”, as used herein, is intended to include DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA.

Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In one embodiment of all aspects of the invention, nucleic acid such as nucleic acid encoding a vaccine peptide or protein, or nucleic acid encoding a binding agent is expressed in cells of the subject treated to provide the vaccine peptide or protein or binding agent. In one embodiment of all aspects of the invention, the nucleic acid is transiently expressed in cells of the subject. Thus, in one embodiment, the nucleic acid is not integrated into the genome of the cells. In one embodiment of all aspects of the invention, the nucleic acid is RNA, preferably in vitro transcribed RNA.

The nucleic acids described herein may be recombinant and/or isolated molecules.

In the present disclosure, the term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA.

In certain embodiments of the present disclosure, the RNA is messenger RNA (mRNA) that relates to a RNA transcript which encodes a peptide or protein. As established in the art, mRNA generally contains a 5′ untranslated region (5′-UTR), a peptide coding region and a 3′ untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

In one embodiment, RNA is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In one embodiment, the RNA described herein may have modified nucleosides. In some embodiments, the RNA comprises a modified nucleoside in place of at least one (e.g., every) uridine.

The term “uracil,” as used herein, describes one of the nucleobases that can occur in the nucleic acid of RNA. The structure of uracil is:

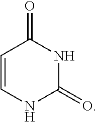

The term “uridine,” as used herein, describes one of the nucleosides that can occur in RNA. The structure of uridine is:

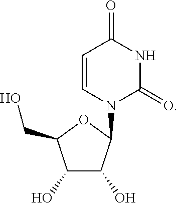 [SMILES: O=c1ccn([C@@H]2O[C@H](CO)[C@@H](O)[C@H]2O)c(=O)[nH]1]

UTP (uridine 5′-triphosphate) has the following structure:

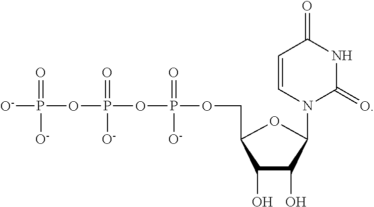

Pseudo-UTP (pseudouridine 5′-triphosphate) has the following structure:

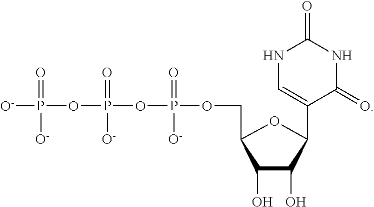

“Pseudouridine” is one example of a modified nucleoside that is an isomer of uridine, where the uracil is attached to the pentose ring via a carbon-carbon bond instead of a nitrogen-carbon glycosidic bond.
Another exemplary modified nucleoside is N1-methyl-pseudouridine (m1Ψ), which has the structure:

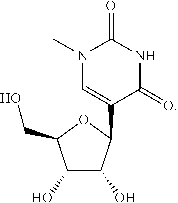 [SMILES: Cn1cc([C@@H]2O[C@H](CO)[C@@H](O)[C@H]2O)c(=O)[nH]c1=O]

N1-methyl-pseudo-UTP has the following structure:

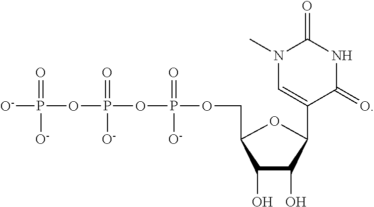

Another exemplary modified nucleoside is 5-methyl-uridine (m5U), which has the structure:

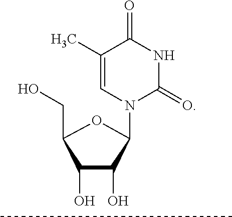

In some embodiments, one or more uridine in the RNA described herein is replaced by a modified nucleoside. In some embodiments, the modified nucleoside is a modified uridine.

In some embodiments, RNA comprises a modified nucleoside in place of at least one uridine. In some embodiments, RNA comprises a modified nucleoside in place of each uridine.

In some embodiments, the modified nucleoside is independently selected from pseudouridine (Ψ), N1-methyl-pseudouridine (m1 Ψ), and 5-methyl-uridine (m5U). In some embodiments, the modified nucleoside comprises pseudouridine (Ψ). In some embodiments, the modified nucleoside comprises N1-methyl-pseudouridine (m1Ψ). In some embodiments, the modified nucleoside comprises 5-methyl-uridine (m5U). In some embodiments, RNA may comprise more than one type of modified nucleoside, and the modified nucleosides are independently selected from pseudouridine (Ψ), N1-methyl-pseudouridine (m1 iv), and 5-methyl-uridine (m5U). In some embodiments, the modified nucleosides comprise pseudouridine (ip) and N1-methyl-pseudouridine (m1Ψ). In some embodiments, the modified nucleosides comprise pseudouridine (ip) and 5-methyl-uridine (m5U). In some embodiments, the modified nucleosides comprise N1-methyl-pseudouridine (m1 Ψ) and 5-methyl-uridine (m5U). In some embodiments, the modified nucleosides comprise pseudouridine (Ψ), N1-methyl-pseudouridine (m1Ψ), and 5-methyl-uridine (m5U).

In some embodiments, the modified nucleoside replacing one or more uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (TM⁵U), 1-taurinomethyl-pseudouridine, 5-taurinomethyl-2-thio-uridine(rm5s2U), 1-taurinomethyl-4-thio-pseudouridine), 5-methyl-2-thio-uridine (m⁵s²U), 1-methyl-4-thio-pseudouridine (m¹s⁴Ψ), 4-thio-1-methyl-pseudouridine, 3-methyl-pseudouridine (m³Ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ Ψ), 5-(isopentenylaminomethyl)uridine (inm⁵U), 5-(isopentenylaminomethyl)-2-thio-uridine (inm⁵s²U), α-thio-uridine, 2¹-O-methyl-uridine (Um), 5,2¹-O-dimethyl-uridine (m⁵Um), 2¹-O-methyl-pseudouridine (Ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2¹-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, the RNA according to the present disclosure comprises a 5′-cap. In one embodiment, the RNA of the present disclosure does not have uncapped 5′-triphosphates. In one embodiment, the RNA may be modified by a 5′- cap analog. The term “5¹-cap” refers to a structure found on the 5′-end of an mRNA molecule and generally consists of a guanosine nucleotide connected to the mRNA via a 5′- to 5′-triphosphate linkage. In one embodiment, this guanosine is methylated at the 7-position. Providing an RNA with a 5′-cap or 5′-cap analog may be achieved by in vitro transcription, in which the 5′-cap is co-transcriptionally expressed into the RNA strand, or may be attached to RNA post-transcriptionally using capping enzymes.

In some embodiments, the building block cap for RNA is m₂^{7,3′-O} Gppp(m₁^{2′-O})ApG (also sometimes referred to as m₂^{7,3′O}G(5′)ppp(5′)m^{2′-O} ApG), which has the following structure:

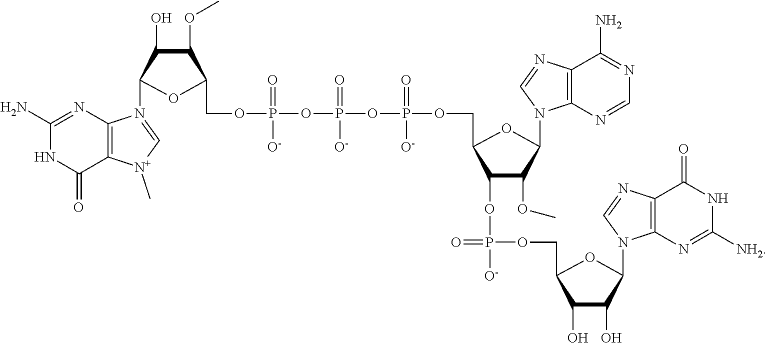

Below is an exemplary Gaol RNA, which comprises RNA and M₂^{7,3′O}G(5′)ppp(5′)m^{2′-O}ApG:

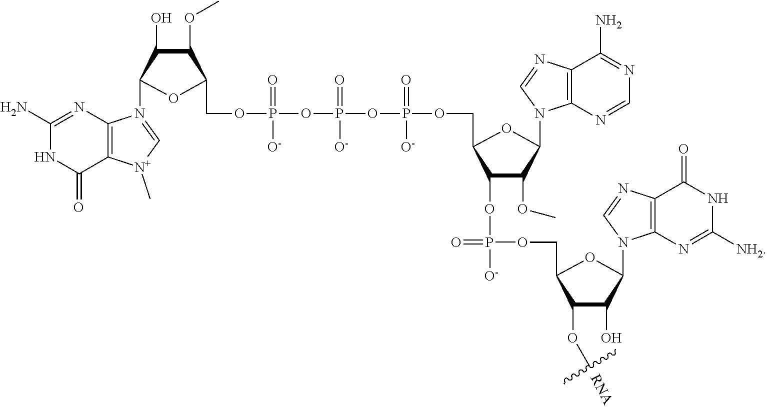

Below is another exemplary Gaol RNA (no cap analog):

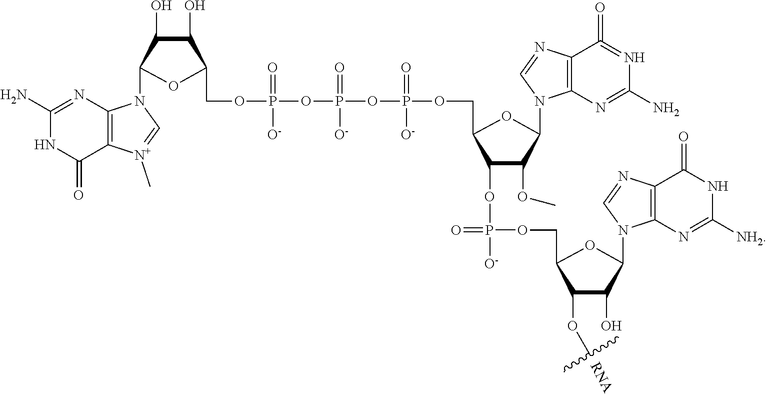

In some embodiments, the RNA is modified with “Cap0” structures using, in one embodiment, the cap analog anti-reverse cap (ARCA Cap (m₂^{7,3′O}G(5′)ppp(5′)G)) with the structure:

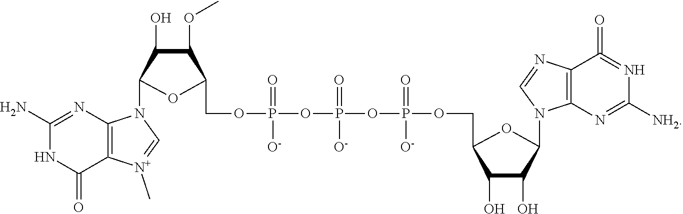

Below is an exemplary Cap0 RNA comprising RNA and M₂^{7,3′O}G(5′)ppp(5′)G:

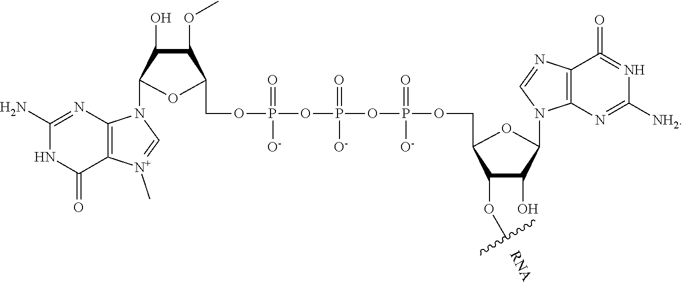

In some embodiments, the “Cap0” structures are generated using the cap analog Beta-S-ARCA (m₂^{7,2′O}G(5^{,})ppSp(5′)G) with the structure:

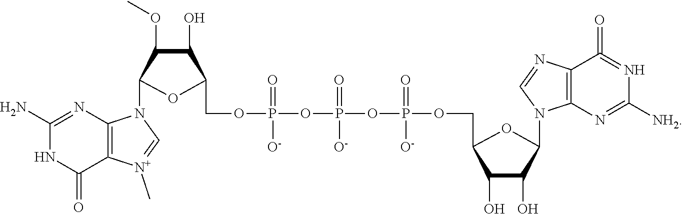

Below is an exemplary Cap0 RNA comprising Beta-S-ARCA (m₂^{7,2′O}G(5′)ppSp(5′)G) and RNA:

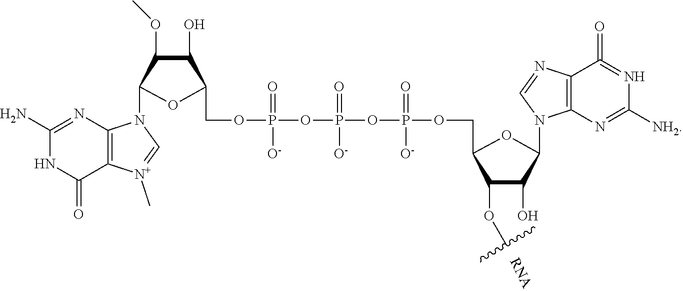

In some embodiments, RNA according to the present disclosure comprises a 5′-UTR and/or a 3′-UTR.

The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end, upstream of the start codon of a protein-encoding region. A 5′-UTR is downstream of the 5′-cap (if present), e.g. directly adjacent to the 5′-cap. A 3′-UTR, if present, is located at the 3′ end, downstream of the termination codon of a protein-encoding region, but the term “3′-UTR” does preferably not include the poly(A) sequence. Thus, the 3′-UTR is upstream of the poly(A) sequence (if present), e.g. directly adjacent to the poly(A) sequence.

In some embodiments, the RNA according to the present disclosure comprises a 3′-poly(A) sequence. As used herein, the term “poly(A) sequence” or “poly-A tail” refers to an uninterrupted or interrupted sequence of adenylate residues which is typically located at the 3′ end of an RNA molecule. Poly(A) sequences are known to those of skill in the art and may follow the 3′ UTR in the RNAs described herein. The poly(A) sequence may be of any length. In some embodiments, a poly(A) sequence comprises or consists of at least 20, at least 30, at least 40, at least 80, or at least 100 and up to 500, up to 400, up to 300, up to 200, or up to 150 nucleotides, and, in particular, about 110 nucleotides. In some embodiments, the poly(A) sequence only consists of A nucleotides. In some embodiments, the poly(A) sequence essentially consists of A nucleotides, but is interrupted by a random sequence of the four nucleotides (A, C, G, and U), as disclosed in WO 2016/005324 A1, hereby incorporated by reference. Such random sequence may be 5 to 50, 10 to 30, or 10 to 20 nucleotides in length. A poly(A) cassette present in the coding strand of DNA that essentially consists of dA nucleotides, but is interrupted by a random sequence having an equal distribution of the four nucleotides (dA, dC, dG, dT) and having a length of e.g. 5 to 50 nucleotides shows, on DNA level, constant propagation of plasmid DNA in E. coli and is still associated, on RNA level, with the beneficial properties with respect to supporting RNA stability and translational efficiency. In some embodiments, no nucleotides other than A nucleotides flank a poly(A) sequence at its 3′ end, i.e., the poly(A) sequence is not masked or followed at its 3′ end by a nucleotide other than A.

In the context of the present disclosure, the term “transcription” relates to a process, wherein the genetic code in a DNA sequence is transcribed into RNA. Subsequently, the RNA may be translated into peptide or protein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Similarly, RNA such as mRNA encodes a protein if translation of the RNA produces the protein in a cell or other biological system.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence.

As used herein, the terms “linked,” “fused”, or “fusion” are used interchangeably. These terms refer to the joining together of two or more elements or components or domains.

### Antigen

The term “antigen” relates to an agent comprising an epitope against which an immune response or an immune effector molecule such as antibody is directed and/or is to be directed. The term “antigen” includes, in particular, proteins and peptides. In one embodiment, an antigen is a disease-associated antigen, such as a tumor antigen.

The term “disease-associated antigen” is used in its broadest sense to refer to any antigen associated with a disease which preferably contains an epitope that will stimulate a host's immune system to make a cellular antigen-specific immune response and/or a humoral antibody response against the disease. The disease-associated antigen, an epitope thereof, or an agent, such as peptide or protein inducing an immune response, targeting the disease-associated antigen or epitope may therefore be used for therapeutic purposes, in particular for vaccination. Disease-associated antigens may be associated with infection by microbes, typically microbial antigens, or associated with cancer, typically tumors.

The term “tumor antigen” refers to a constituent of cancer cells which may be derived from the cytoplasm, the cell surface and the cell nucleus. In particular, it refers to those antigens which are produced intracellularly or as surface antigens on tumor cells. A tumor antigen is typically expressed preferentially by cancer cells (e.g., it is expressed at higher levels in cancer cells than in non-cancer cells) and in some instances it is expressed solely by cancer cells. Examples of tumor antigens include, without limitation, p53, ART-4, BAGE, beta-catenin/m, Bcr-abL CAMEL, CAP-1 , CASP-8, CDC27/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1 , MAGE-A2, MAGE- A3, MAGE-A4, MAGE- A5, MAGE-A6, MAGE-A7, MAGE-A8, MAGE-A9, MAGE-A 10, MAGE-A 1 1, or MAGE- Al2, MAGE-B, MAGE-C, MART- 1 /Melan-A, MC1R, Myosin/m, MUC1 , MUM-1 , MUM -2, MUM -3, NA88-A, NF1 , NY-ES0-1 , NY-BR-1 , p190 minor BCR-abL, Pml/RARa, PRAME, proteinase 3, PSA, PSM, RAGE, RU1 or RU2, SAGE, SART-1 or SART-3, SCGB3A2, SCP1 , SCP2, SCP3, SSX, SURVIVIN, TEL/AML1 , TPI/m, TRP-1 , TRP-2, TRP-2/INT2, TPTE, WT, and WT-1.

As used herein, “tumor antigen” or “cancer antigen” includes (i) tumor-specific antigens, (ii) tumor-associated antigens, (iii) embryonic antigens on tumors, (iv) tumor-specific membrane antigens, (v) tumor-associated membrane antigens, (vi) growth factor receptors, and (xi) any other type of antigen or material that is associated with a cancer.

Any tumor antigen (preferably expressed by a tumor cell) can be targeted by the vaccination disclosed herein. In one embodiment, the tumor antigen is presented by a tumor cell and thus can be targeted by T cells. Vaccination as disclosed herein preferably activates T cells specific for MHC presented tumor antigens. The tumor antigen may be a tumor-specific antigen (TSA) or a tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

The term “viral antigen” refers to any viral component having antigenic properties, i.e. being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The term “expressed on the cell surface”, “associated with the cell surface” or a similar term means that a molecule such as an antigen is associated with and located at the plasma membrane of a cell, wherein at least a part of the molecule faces the extracellular space of said cell and is accessible from the outside of said cell, e.g., by antibodies located outside the cell. In this context, a part is preferably at least 4, preferably at least 8, preferably at least 12, more preferably at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules.

The term “extracellular portion” or “exodomain” in the context of the present invention refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

The term “epitope” refers to an antigenic determinant in a molecule, i.e., to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 10 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes B cell epitopes and T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

The peptide and protein antigens suitable for use according to the disclosure typically include a peptide or protein comprising an epitope for inducing an immune response. The peptide or protein or epitope may be derived from a target antigen, i.e. the antigen against which an immune response is to be elicited. For example, the peptide or protein antigen or the epitope contained within the peptide or protein antigen may be a target antigen or a fragment or variant of a target antigen.

A peptide or protein antigen, either administered per se or in the form of RNA encoding the peptide or protein antigen, i.e., a vaccine antigen, preferably results in stimulation, priming and/or expansion of T cells in the treated subject. Said stimulated, primed and/or expanded T cells are preferably directed against a target antigen, in particular a target antigen expressed by diseased cells, tissues and/or organs, i.e., a disease-associated antigen. Thus, a vaccine antigen may comprise the disease-associated antigen, or a fragment or variant thereof. In one embodiment, such fragment or variant is immunologically equivalent to the disease-associated antigen. In the context of the present disclosure, the term “fragment of an antigen” or “variant of an antigen” means an agent which results in stimulation, priming and/or expansion of T cells which stimulated, primed and/or expanded T cells target the antigen, i.e. a disease-associated antigen, in particular when presented by diseased cells, tissues and/or organs. Thus, the vaccine antigen may correspond to or may comprise the disease-associated antigen, may correspond to or may comprise a fragment of the disease-associated antigen or may correspond to or may comprise an antigen which is homologous to the disease-associated antigen or a fragment thereof. If the vaccine antigen comprises a fragment of the disease-associated antigen or an amino acid sequence which is homologous to a fragment of the disease-associated antigen said fragment or amino acid sequence may comprise an epitope such as a T cell epitope of the disease-associated antigen or a sequence which is homologous to an epitope such as a T cell epitope of the disease-associated antigen. Thus, according to the disclosure, a vaccine antigen may comprise an immunogenic fragment of a disease-associated antigen or an amino acid sequence being homologous to an immunogenic fragment of a disease-associated antigen. An “immunogenic fragment of an antigen” according to the disclosure preferably relates to a fragment of an antigen which is capable of stimulating, priming and/or expanding T cells when presented in the context of MHC molecules. It is preferred that the vaccine antigen (similar to the disease-associated antigen) can be presented by a cell such as an antigen-presenting cell so as to provide the relevant epitope for binding by T cells. The vaccine antigen may be a recombinant antigen.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants used for immunization. For example, an amino acid sequence is immunologically equivalent to a reference amino acid sequence if said amino acid sequence when exposed to the immune system of a subject such as T cells binding to the reference amino acid sequence or cells expressing the reference amino acid sequence induces an immune reaction having a specificity of reacting with the reference amino acid sequence. Thus, a molecule which is immunologically equivalent to an antigen exhibits the same or essentially the same properties and/or exerts the same or essentially the same effects regarding the stimulation, priming and/or expansion of T cells as the antigen to which the T cells are targeted.

The term “priming” refers to a process wherein a T cell has its first contact with its specific antigen and causes differentiation into effector T cells.

The term “clonal expansion” or “expansion” refers to a process wherein a specific entity is multiplied. In the context of the present disclosure, the term is preferably used in the context of an immunological response in which lymphocytes are stimulated by an antigen, proliferate, and the specific lymphocyte recognizing said antigen is amplified. Preferably, clonal expansion leads to differentiation of the lymphocytes.

In one embodiment, the target antigen is a tumor antigen and the peptide or protein comprising an epitope or a fragment thereof (e.g., an epitope) is derived from the tumor antigen. The tumor antigen may be a “standard” antigen, which is generally known to be expressed in various cancers. The tumor antigen may also be a “neo-antigen”, which is specific to an individual's tumor and has not been previously recognized by the immune system. A neo-antigen or neo-epitope may result from one or more cancer-specific mutations in the genome of cancer cells resulting in amino acid changes. If the tumor antigen is a neo-antigen, the peptide or protein comprising an epitope preferably comprises an epitope or a fragment of said neo-antigen comprising one or more amino acid changes.

Cancer mutations vary with each individual. Thus, cancer mutations that encode novel epitopes (neo-epitopes) represent attractive targets in the development of vaccine compositions and immunotherapies.

The efficacy of tumor immunotherapy relies on the selection of cancer-specific antigens and epitopes capable of inducing a potent immune response within a host. RNA can be used to deliver patient-specific tumor epitopes to a patient. Dendritic cells (DCs) residing in the spleen represent antigen-presenting cells of particular interest for RNA expression of immunogenic epitopes or antigens such as tumor epitopes. The use of multiple epitopes has been shown to promote therapeutic efficacy in tumor vaccine compositions. Rapid sequencing of the tumor mutanome may provide multiple epitopes for individualized vaccines which can be encoded by RNA described herein, e.g., as a single polypeptide wherein the epitopes are optionally separated by linkers. In certain embodiments of the present disclosure, the RNA encodes at least one epitope, at least two epitopes, at least three epitopes, at least four epitopes, at least five epitopes, at least six epitopes, at least seven epitopes, at least eight epitopes, at least nine epitopes, or at least ten epitopes. Exemplary embodiments include RNA that encodes at least five epitopes (termed a “pentatope”) and RNA that encodes at least ten epitopes (termed a “decatope”).

The peptide and protein antigen can be 2-100 amino acids, including for example, at least 5 amino acids, at least 10 amino acids, at least 15 amino acids, at least 20 amino acids, at least 25 amino acids, at least 30 amino acids, at least 35 amino acids, at least 40 amino acids, at least 45 amino acids, or at least 50 amino acids in length. In some embodiments, a peptide can be greater than 50 amino acids. In some embodiments, the peptide can be greater than 100 amino acids.

The peptide or protein antigen can be any peptide or protein that can induce or increase the ability of the immune system to develop antibodies and T cell responses to a target antigen, e.g., disease-associated antigen.

Binding agent

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized.

See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (abbreviated herein as CH or CH). The heavy chain constant region typically is comprised of three domains, CH1, CH2, and CH3. The hinge region is the region between the CH1 and CH2 domains of the heavy chain and is highly flexible. Disulphide bonds in the hinge region are part of the interactions between two heavy chains in an IgG molecule. Each light chain typically is comprised of a light chain variable region (abbreviated herein as V_{L} or VL) and a light chain constant region (abbreviated herein as C_{L} or CL). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Unless otherwise stated or contradicted by context, CDR sequences herein are identified according to IMGT rules using DomainGapAlign (Lefranc MP., Nucleic Acids Research 1999; 27:209-212 and Ehrenmann F., Kaas Q. and Lefranc M.-P. Nucleic Acids Res., 38, D301-307 (2010); see also internet http address www imgt.org/. Unless otherwise stated or contradicted by context, reference to amino acid positions in the constant regions in the present invention is according to the EU-numbering (Edelman et al., Proc Natl Acad Sci U S A. 1969 May; 63(1):78-85; Kabat et al., Sequences of Proteins of Immunological Interest, Fifth Edition. 1991 NIH Publication No. 91-3242). For example, SEQ ID NO: 17 or 18 herein sets forth amino acids positions 118-447 according to EU numbering, of the IgG1 heavy chain constant region.

The term “amino acid corresponding to position . . . ” as used herein refers to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is considered well-known in the art how to align a sequence or segment in a sequence and thereby determine the corresponding position in a sequence to an amino acid position according to the present invention.

The term “binding agent” in the context of the present invention refers to any agent capable of binding to desired antigens. In certain embodiments of the invention, the binding agent is an antibody, antibody fragment, or construct thereof. The binding agent may also comprise synthetic, modified or non-naturally occurring moieties, in particular non-peptide moieties. Such moieties may, for example, link desired antigen-binding functionalities or regions such as antibodies or antibody fragments. In one embodiment, the binding agent is a synthetic construct comprising antigen-binding CDRs or variable regions.

The term “antibody” (Ab) in the context of the present invention refers to an immunoglobulin molecule, a fragment of an immunoglobulin molecule, or a derivative of either thereof, which has the ability to bind, preferably specifically bind to an antigen. In one embodiment, binding takes place under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The term “antigen-binding region”, as used herein, refers to the region which interacts with the antigen and typically comprises both a VH region and a VL region. The term antibody when used herein comprises not only monospecific antibodies, but also multispecific antibodies which comprise multiple, such as two or more, e.g. three or more, different antigen-binding regions. The constant regions of the antibodies (Abs) may mediate the binding of the immunoglobulin to host tissues or factors, including various cells of the immune system (such as effector cells) and components of the complement system such as C1 q, the first component in the classical pathway of complement activation. As indicated above, the term antibody as used herein, unless otherwise stated or clearly contradicted by context, includes fragments of an antibody that are antigen-binding fragments, i.e., retain the ability to specifically bind to the antigen, and antibody derivatives, i.e., constructs that are derived from an antibody. It has been shown that the antigen-binding function of an antibody may be performed by fragments of a full-length antibody. Examples of antigen-binding fragments encompassed within the term “antibody” include (i) a Fab' or Fab fragment, a monovalent fragment consisting of the VL, VH, CL and CH1 domains, or a monovalent antibody as described in WO2007059782 (Genmab); (ii) F(ab ′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 341, 544-546 (1989)), which consists essentially of a VH domain and also called domain antibodies (Holt et al; Trends Biotechnol. 2003 Nov;21(11):484-90); (vi) camelid or Nanobody molecules (Revets et al; Expert Opin Biol Ther. 2005 Jan;5(1):111-24) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention, as well as bispecific formats of such fragments, are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques.

An antibody as generated can possess any isotype. As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody of the invention may be a sequence variant of a naturally-occurring IgG1 antibody, including variations in the constant regions.

The term “monoclonal antibody” as used herein refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

The term “bispecific antibody” or “bs” in the context of the present invention refers to an antibody having two different antigen-binding regions defined by different antibody sequences. In some embodiments, said different antigen-binding regions bind different epitopes on the same antigen. However, in preferred embodiments, said different antigen-binding regions bind different target antigens. A bispecific antibody can be of any format, including any of the bispecific antibody formats described herein below.

When used herein, unless contradicted by context, the term “Fab-arm” or “arm” includes one heavy chain-light chain pair and is used interchangeably with “half-molecule” herein.

When a bispecific antibody is described to comprise a half-molecule antibody “derived from” a first antibody, and a half-molecule antibody “derived from” a second antibody, the term “derived from” indicates that the bispecific antibody was generated by recombining, by any known method, said half-molecules from each of said first and second antibodies into the resulting bispecific antibody. In this context, “recombining” is not intended to be limited by any particular method of recombining and thus includes all of the methods for producing bispecific antibodies described herein below, including for example recombining by half-molecule exchange, as well as recombining at nucleic acid level and/or through co-expression of two half-molecules in the same cells.

The term “monovalent antibody” means in the context of the present invention that an antibody molecule is capable of binding a single molecule of an antigen, and thus is not capable of crosslinking antigens or cells.

The term “full-length” when used in the context of an antibody indicates that the antibody is not a fragment, but contains all of the domains of the particular isotype normally found for that isotype in nature, e.g. the VH, CH1, CH2, CH3, hinge, VL and CL domains for an IgG1 antibody.

When used herein, unless contradicted by context, the term “Fc region” refers to an antibody region consisting of the two Fc sequences of the heavy chains of an immunoglobulin, wherein said Fc sequences comprise at least a hinge region, a CH2 domain, and a CH3 domain.

When used herein, the term “heterodimeric interaction between the first and second CH3 regions” refers to the interaction between the first CH3 region and the second CH3 region in a first-CH3/second-CH3 heterodimeric protein.

When used herein, the term “homodimeric interactions of the first and second CH3 regions” refers to the interaction between a first CH3 region and another first CH3 region in a first-CH3/first-CH3 homodimeric protein and the interaction between a second CH3 region and another second CH3 region in a second-CH3/second-CH3 homodimeric protein.

As used herein, the terms “binding” or “capable of binding” in the context of the binding of an antibody to a predetermined antigen or epitope typically is a binding with an affinity corresponding to a K_{D} of about 10⁻⁷ M or less, such as about 10⁻⁸M or less, such as about 10⁻⁸ M or less, about 10⁻¹⁰ M or less, or about 10⁻¹¹ M or even less, when determined using Bio-Layer Interferometry (BLI), or, for instance, when determined using surface plasmon resonance (SPR) technology in a BlAcore 3000 instrument using the antigen as the ligand and the antibody as the analyte. The antibody binds to the predetermined antigen with an affinity corresponding to a KD that is at least ten-fold lower, such as at least 100-fold lower, for instance at least 1,000-fold lower, such as at least 10,000-fold lower, for instance at least 100,000-fold lower than its affinity for binding to a non-specific antigen (e.g., BSA, casein) other than the predetermined antigen or a closely-related antigen. The amount with which the affinity is lower is dependent on the KD of the antibody, so that when the Ko of the antibody is very low (that is, the antibody is highly specific), then the degree to which the affinity for the antigen is lower than the affinity for a non-specific antigen may be at least 10,000-fold.

The term “k_{d}” (sec⁻¹), as used herein, refers to the dissociation rate constant of a particular antibody-antigen interaction. Said value is also referred to as the koff value.

The term “K_{D}” (M), as used herein, refers to the dissociation equilibrium constant of a particular antibody-antigen interaction.

In a preferred embodiment, the antibody described herein is isolated. An “isolated antibody” as used herein, is intended to refer to an antibody which is substantially free of other antibodies having different antigenic specificities. In a preferred embodiment, an isolated bispecific antibody that specifically binds to PD-L1 and CD137 is substantially free of monospecific antibodies that specifically bind to PD-L1 or CD137.

The term “PD-L1” when used herein, refers to the Programmed Death-Ligand 1 protein. PD-L1 is found in humans and other species, and thus, the term “PD-L1” is not limited to human PD-L1 unless contradicted by context. Human, macaque, African elephant, wild boar and mouse PD-L1 sequences can be found through Genbank accession no. NP_054862.1, XP_005581836, XP_003413533, XP_005665023 and NP_068693, respectively. The sequence of human PD-L1 is also shown in SEQ ID NO: 21, wherein amino acids 1-18 are predicted to be a signal peptide.

The term “PD-L2” when used herein, refers to the human Programmed Death 1-Ligand 2 protein (Genbank accession no. NP_079515).

The term “PD-1” when used herein, preferably refers to the human Programmed Death-1 protein, also known as CD279.

The term “CD137” as used herein, refers to the Cluster of Differentiation 137 protein, preferably human CD137. CD137 (4-1BB), also referred to as TNFRSF9, is the receptor for the ligand TNFSF9/4-1BBL. CD137 is believed to be involved in T cell activation. In one embodiment, CD137 is human CD137, having UniProt accession number Q07011. The sequence of human CD137 is also shown in SEQ ID NO: 22, wherein amino acids 1-23 are predicted to be a signal peptide.

A “PD-L1 antibody” or “anti-PD-L1 antibody” is an antibody as described above, which binds specifically to the antigen PD-L1, in particular human PD-L1.

A “CD137 antibody” or “anti-CD137 antibody” is an antibody as described above, which binds specifically to the antigen CD137.

A “CD137xPD-L1 antibody”, “anti-CD137xPD-L1 antibody”, “PD-L1xCD137 antibody” or “anti-PD-L1xCD137 antibody” is a bispecific antibody, which comprises two different antigen-binding regions, one of which binds specifically to the antigen PD-L1 and one of which binds specifically to CD137.

The present invention also envisions antibodies comprising functional variants of the VL regions, VH regions, or one or more CDRs of the antibodies described herein. A functional variant of a VL, VH, or CDR used in the context of an antibody still allows the antibody to retain at least a substantial proportion (at least about 50%, 60%, 70%, 80%, 90%, 95% or more) of the affinity and/or the specificity/selectivity of the “reference” or “parent” antibody and in some cases, such an antibody may be associated with greater affinity, selectivity and/or specificity than the parent antibody.

Such functional variants typically retain significant sequence identity to the parent antibody. The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm.

Exemplary variants include those which differ from VH and/or VL and/or CDR regions of the parent antibody sequences mainly by conservative substitutions; for instance, 10, such as 9, 8, 7, 6, 5, 4, 3, 2 or 1 of the substitutions in the variant are conservative amino acid residue replacements.

Functional variants of antibody sequences described herein such as VL regions, or VH regions, or antibody sequences having a certain degree of homology or identity to antibody sequences described herein such as VL regions, or VH regions preferably comprise modifications or variations in the non-CDR sequences, while the CDR sequences preferably remain unchanged.

In the context of the present invention, the following notations are, unless otherwise indicated, used to describe a mutation: i) substitution of an amino acid in a given position is written as e.g. K409R which means a substitution of a lysine in position 409 of the protein with an arginine; and ii) for specific variants the specific three or one letter codes are used, including the codes Xaa and X to indicate any amino acid residue. Thus, the substitution of lysine with arginine in position 409 is designated as: K409R, and the substitution of lysine with any amino acid residue in position 409 is designated as K409X. In case of deletion of lysine in position 409 it is indicated by K409*.

In the context of the present invention, “inhibition of PD-L1 binding to PD-1” refers to any detectably significant reduction in the binding of PD-L1 to PD-1 in the presence of an antibody capable of binding PD-L1. Typically, inhibition means an at least about 10% reduction, such as an at least about 15%, e.g. an at least about 20%, such as an at least 40% reduction in binding between PD-L1 and PD-1, caused by the presence of an anti-PD-L1 antibody. Inhibition of PD-L1 binding to PD-1 may be determined by any suitable technique.

The term “specificity” as used herein is intended to have the following meaning unless contradicted by context. Two antibodies have the “same specificity” if they bind to the same antigen and the same epitope.

The term “chimeric antibody” as used herein, refers to an antibody wherein the variable region is derived from a non-human species (e.g. derived from rodents) and the constant region is derived from a different species, such as human. Chimeric monoclonal antibodies for therapeutic applications are developed to reduce antibody immunogenicity. The terms “variable region” or “variable domain” as used in the context of chimeric antibodies, refer to a region which comprises the CDRs and framework regions of both the heavy and light chains of the immunoglobulin. Chimeric antibodies may be generated by using standard DNA techniques as described in Sambrook et al., 1989, Molecular Cloning: A laboratory Manual, New York: Cold Spring Harbor Laboratory Press, Ch. 15. The chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see W092/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “human antibody” as used herein, refers to antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse or rat, have been grafted onto human framework sequences. Human monoclonal antibodies can be produced by a variety of techniques, including conventional monoclonal antibody methodology, e.g., the standard somatic cell hybridization technique of Kohler and Milstein, Nature 256: 495 (1975). Although somatic cell hybridization procedures are preferred, in principle, other techniques for producing monoclonal antibody can be employed, e.g., viral or oncogenic transformation of B-lymphocytes or phage display techniques using libraries of human antibody genes. A suitable animal system for preparing hybridomas that secrete human monoclonal antibodies is the murine system. Hybridoma production in the mouse is a very well established procedure. Immunization protocols and techniques for isolation of immunized splenocytes for fusion are known in the art. Fusion partners (e.g., murine myeloma cells) and fusion procedures are also known. Human monoclonal antibodies can thus e.g. be generated using transgenic or transchromosomal mice or rats carrying parts of the human immune system rather than the mouse or rat system. Accordingly, in one embodiment, a human antibody is obtained from a transgenic animal, such as a mouse or a rat, carrying human germline immunoglobulin sequences instead of animal immunoglobulin sequences. In such embodiments, the antibody originates from human germline immunoglobulin sequences introduced in the animal, but the final antibody sequence is the result of said human germline immunoglobulin sequences being further modified by somatic hypermutations and affinity maturation by the endogeneous animal antibody machinery, see e.g. Mendez et al. 1997 Nat Genet. 15(2):146-56.

The term “reducing conditions” or “reducing environment” refers to a condition or an environment in which a substrate, here a cysteine residue in the hinge region of an antibody, is more likely to become reduced than oxidized.

The term “recombinant host cell” (or simply “host cell”), as used herein, is intended to refer to a cell into which an expression vector has been introduced, e.g. an expression vector encoding an antibody of the invention. Recombinant host cells include, for example, transfectomas, such as CHO, CHO-S, HEK, HEK293, HEK-293F, Expi293F, PER.C6 or NSO cells, and lymphocytic cells.

The term “anti-idiotypic antibody” refers to an antibody which recognizes unique determinants generally associated with the antigen-binding site of an antibody.

The term “competes” and “competition” refers to the competition between a first antibody and a second antibody to the same antigen. Alternatively “competes” and “competition” may also refer to the competition between an antibody and an endogenous ligand for binding to the corresponding receptor of the endogenous ligand. If an antibody prevents the binding of the endogenous ligand to its receptor, such an antibody is said to block the endogenous interaction of the ligand with its receptor and therefore is competing with the endogenous ligand. It is well known to a person skilled in the art how to test for competition of antibodies for binding to a target antigen. An example of such a method is a so-called cross-competition assay, which may e.g. be performed as an ELISA or by flow-cytometry. Alternatively, competition may be determined using biolayer interferometry.

Antibodies which compete for binding to a target antigen binding may bind different epitopes on the antigen, wherein the epitopes are so close to each other that a first antibody binding to one epitope prevents binding of a second antibody to the other epitope. In other situations, however, two different antibodies may bind the same epitope on the antigen and would compete for binding in a competition binding assay. Such antibodies binding to the same epitope are considered to have the same specificity herein. Thus, in one embodiment, antibodies binding to the same epitope are considered to bind to the same amino acids on the target molecule. That antibodies bind to the same epitope on a target antigen may be determined by standard alanine scanning experiments or antibody-antigen crystallization experiments known to a person skilled in the art.

As described above, various formats of antibodies have been described in the art. The binding agent of the invention can in principle be an antibody of any isotype. The choice of isotype typically will be guided by the desired Fc-mediated effector functions, such as ADCC induction, or the requirement for an antibody devoid of Fc-mediated effector function (“inert” antibody). Exemplary isotypes are IgG1, IgG2, IgG3, and IgG4. Either of the human light chain constant regions, kappa or lambda, may be used. The effector function of the antibodies of the present invention may be changed by isotype switching to, e.g., an IgG1, IgG2, IgG3, IgG4, IgD, IgA, IgE, or IgM antibody for various therapeutic uses. In one embodiment, both heavy chains of an antibody of the present invention are of the IgG1 isotype, for instance an IgG1,K. Optionally, the heavy chain may be modified in the hinge and/or CH3 region as described elsewhere herein.

Preferably, each of the antigen-binding regions comprises a heavy chain variable region (VH) and a light chain variable region (VL), and wherein said variable regions each comprise three CDR sequences, CDR1, CDR2 and CDR3, respectively, and four framework sequences, FR1, FR2, FR3 and FR4, respectively. Furthermore, preferably, the antibody comprises two heavy chain constant regions (CH), and two light chain constant regions (CL).

In one embodiment of the invention, the binding agent is a full-length antibody, such as a full-length IgG1 antibody. In another embodiment, the antibody is a full-length IgG4 antibody, preferably with a stabilized hinge region. Modifications that stabilize the IgG4 hinge region, such as the S228P mutation in the core hinge, have been described in the art, see e.g. Labrijn et al., 2009 Nat Biotechnol. 27(8):767-71.

In other embodiments of the invention, the binding agent of the invention comprises an antibody fragment, such as a Fab' or Fab fragment, a monovalent fragment consisting of the VL, VH, CL and CH1 domains, a monovalent antibody as described in WO2007059782 (Genmab), a F(ab′)2 fragment, a Fd fragment, a Fv fragment, a dAb fragment, camelid or nanobodies, or an isolated complementarity determining region (CDR).

Binding agents of the invention are preferably human, humanized or chimeric antibodies. In embodiments, wherein the antibody is a bispecific antibody, both half-molecules can be human, humanized or chimeric, or the half-molecules can differ in character with respect to sequence origin.

For example, in one embodiment, the binding agent, e.g. a bispecific antibody, comprises two half-molecules each comprising an antigen-binding region. Preferably, the half-molecule comprising the antigen-binding region capable of binding to human PD-L1 is human and the half-molecule comprising the antigen-binding region capable of binding to human CD137 is humanized.

Many different formats and uses of bispecific antibodies are known in the art, and were reviewed by Kontermann; Drug Discov Today, 2015 July; 20(7):838-47 and; MAbs, 2012 Mar-Apr;4(2):182-97. A bispecific antibody according to the present invention is not limited to any particular bispecific format or method of producing it.

Examples of bispecific antibody molecules which may be used in the present invention comprise (i) a single antibody that has two arms comprising different antigen-binding regions; (ii) a single chain antibody that has specificity to two different epitopes, e.g., via two scFvs linked in tandem by an extra peptide linker; (iii) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody

Engineering, Springer Berlin Heidelberg (2010)); (iv) a chemically-linked bispecific (Fab′)2 fragment; (v) a Tandab, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody.

In one embodiment of the invention, the binding agent of the present invention is a diabody or a cross-body. In one embodiment, the binding agent of the invention is a bispecific antibody obtained via a controlled Fab-arm exchange (such as described in WO2011131746 (Genmab)).

Examples of different classes of binding agents according to the present invention include but are not limited to (i) IgG-like molecules with complementary CH3 domains to force heterodimerization; (ii) recombinant IgG-like dual targeting molecules, wherein the two sides of the molecule each contain the Fab fragment or part of the Fab fragment of at least two different antibodies; (iii) IgG fusion molecules, wherein full length IgG antibodies are fused to extra Fab fragment or parts of Fab fragment; (iv) Fc fusion molecules, wherein single chain Fv molecules or stabilized diabodies are fused to heavy-chain constant-domains, Fc regions or parts thereof; (v) Fab fusion molecules, wherein different Fab-fragments are fused together, fused to heavy-chain constant-domains, Fc regions or parts thereof; and (vi) ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule fused to heavy-chain constant-domains, Fc regions or parts thereof.

Examples of IgG-like molecules with complementary CH3 domain molecules include but are not limited to the Triomab/Quadroma molecules (Trion Pharma/Fresenius Biotech; Roche, WO2011069104), the so-called Knobs-into-Holes molecules (Genentech, WO9850431), CrossMAbs (Roche, WO2011117329) and the electrostatically-matched molecules (Amgen, EP1870459 and WO2009089004; Chugai, US201000155133; Oncomed, WO2010129304), the LUZ-Y molecules (Genentech, Wranik et al. J. Biol. Chem. 2012, 287(52): 43331-9, doi: 10.1074/jbc.M112.397869. Epub 2012 Nov 1), DIG-body and PIG-body molecules (Pharmabcine, WO2010134666, WO2014081202), the Strand Exchange Engineered Domain body (SEEDbody) molecules (EMD Serono, WO2007110205), the Biclonics molecules (Merus, W02013157953), FcAAdp molecules (Regeneron, WO201015792), bispecific IgG1 and IgG2 molecules (Pfizer/Rinat, WO11143545), Azymetric scaffold molecules (Zymeworks/Merck, WO2012058768), mAb-Fv molecules (Xencor, WO2011028952), bivalent bispecific antibodies (WO2009080254) and the DuoBody® molecules (Genmab, WO2011131746).

Examples of recombinant IgG-like dual targeting molecules include but are not limited to Dual Targeting (DT)-Ig molecules (WO2009058383), Two-in-one Antibody (Genentech; Bostrom, et al 2009. Science 323, 1610-1614.), Cross-linked Mabs (Karmanos Cancer Center), mAb2 (F-Star, WO2008003116), Zybody molecules (Zyngenia; LaFleur et al. MAbs. 2013 March-April; 5(2):208-18), approaches with common light chain (Crucell/Merus, US7,262,028), KABodies (Novlmmune, WO2012023053) and CovX-body (CovX/Pfizer; Doppalapudi, V.R., et al 2007. Bioorg. Med. Chem. Lett. 17,501-506.). Examples of IgG fusion molecules include but are not limited to Dual Variable Domain (DVD)-Ig molecules (Abbott, U.S. Pat. No. 7,612,181), Dual domain double head antibodies (Unilever; Sanofi Aventis, WO20100226923), IgG-like Bispecific molecules (ImClone/Eli Lilly, Lewis et al. Nat Biotechnol. 2014 February; 32(2):191-8), Ts2Ab (Med Immune/AZ; Dimasi et al. J Mol Biol. 2009 Oct. 30; 393(3):672-92) and BsAb molecules (Zymogenetics, WO2010111625), HERCULES molecules (Biogen Idec, US007951918), scFv fusion molecules (Novartis), scFv fusion molecules (Changzhou Adam Biotech Inc, CN 102250246) and TvAb molecules (Roche, WO2012025525, WO2012025530).

Examples of Fc fusion molecules include but are not limited to ScFv/Fc Fusions (Pearce et al., Biochem Mol Biol Int. 1997 September; 42(6):1179-88), SCORPION molecules (Emergent BioSolutions/Trubion, Blankenship JW, et al. AACR 100th Annual meeting 2009 (Abstract # 5465); Zymogenetics/BMS, WO2010111625), Dual Affinity Retargeting Technology (Fc-DART) molecules (MacroGenics, WO2008157379, W02010080538) and Dual(ScFv)2-Fab molecules (National Research Center for Antibody Medicine—China).

Examples of Fab fusion bispecific antibodies include but are not limited to F(ab)2 molecules (Medarex/AMGEN; Deo et al J lmmunol. 1998 Feb. 15;160(4):1677-86.), Dual-Action or Bis-Fab molecules (Genentech, Bostrom, et al 2009. Science 323, 1610-1614.), Dock-and-Lock (DNL) molecules (ImmunoMedics, WO2003074569, WO2005004809), Bivalent Bispecific molecules (Biotecnol, Schoonjans, J Immunol. 2000 Dec 15;165(12):7050-7.) and Fab-Fv molecules (UCB-Celltech, WO 2009040562 A1).

Examples of ScFv-, diabody-based and domain antibodies include but are not limited to Bispecific T Cell Engager (BiTE) molecules (Micromet, W02005061547), Tandem Diabody molecules (TandAb) (Affimed) Le Gall et al., Protein Eng Des Sel. 2004 April; 17(4):357-66.), Dual Affinity Retargeting Technology (DART) molecules (MacroGenics, WO2008157379, WO2010080538), Single-chain Diabody molecules (Lawrence, FEBS Lett. 1998 Apr 3;425(3):479-84), TCR-like Antibodies (AIT, ReceptorLogics), Human Serum Albumin ScFv Fusion (Merrimack, W02010059315) and COMBODY molecules (Epigen Biotech, Zhu et al. Immunol Cell Biol. 2010 Aug;88(6):667-75.), dual targeting nanobodies (Ablynx, Hmila et al., FASEB J. 2010) and dual targeting heavy chain only domain antibodies.

In one aspect, the bispecific antibody of the invention comprises a first Fc sequence comprising a first CH3 region, and a second Fc sequence comprising a second CH3 region, wherein the sequences of the first and second CH3 regions are different and are such that the heterodimeric interaction between said first and second CH3 regions is stronger than each of the homodimeric interactions of said first and second CH3 regions. More details on these interactions and how they can be achieved are provided in WO2011131746 and WO2013060867 (Genmab), which are hereby incorporated by reference.

As described further herein, a stable bispecific PD-L1xCD137 antibody can be obtained at high yield using a particular method on the basis of one homodimeric starting PD-L1 antibody and one homodimeric starting CD137 antibody containing only a few, conservative, asymmetrical mutations in the CH3 regions. Asymmetrical mutations mean that the sequences of said first and second CH3 regions contain amino acid substitutions at non-identical positions.

In one embodiment, the bispecific antibody of the invention as defined in any of the embodiments disclosed herein, comprises a first CH3 region which has an amino acid substitution at a position selected from the group consisting of: 366, 368, 370, 399, 405, 407 and 409 in a human IgG1 heavy chain, and a second CH3 region which has an amino acid substitution at a position selected from the group consisting of: 366, 368, 370, 399, 405, 407 and 409 in a human IgG1 heavy chain, and wherein the first and second CH3 regions are not substituted in the same positions.

In one embodiment, the bispecific antibody of the invention as defined in any of the embodiments disclosed herein, comprises a first CH3 region which has an amino acid substitution at position 366 in a human IgG1 heavy chain, and a second CH3 region which has an amino acid substitution at a position selected from the group consisting of: 368, 370, 399, 405, 407 and 409 in a human IgG1 heavy chain. In one embodiment, the amino acid at position 366 in a human IgG1 heavy chain is selected from Ala, Asp, Glu, His, Asn, Val, or Gln.

In one embodiment, the bispecific antibody of the invention as defined in any of the embodiments disclosed herein, comprises a first CH3 region which has an amino acid substitution at position 368 in a human IgG1 heavy chain, and a second CH3 region which has an amino acid substitution at a position selected from the group consisting of: 366, 370, 399, 405, 407 and 409 in a human IgG1 heavy chain. In one embodiment, the bispecific antibody of the invention as defined in any of the embodiments disclosed herein, comprises a first CH3 region which has an amino acid substitution at position 370 in a human IgG1 heavy chain, and a second CH3 region which has an amino acid substitution at a position selected from the group consisting of: 366, 368, 399, 405, 407 and 409 in a human IgG1 heavy chain. In one embodiment, the bispecific antibody of the invention as defined in any of the embodiments disclosed herein, comprises a first CH3 region which has an amino acid substitution at position 399 in a human IgG1 heavy chain, and a second CH3 region which has an amino acid substitution at a position selected from the group consisting of: 366, 368, 370, 405, 407 and 409 in a human IgG1 heavy chain.

In one embodiment, the bispecific antibody of the invention as defined in any of the embodiments disclosed herein, comprises a first CH3 region which has an amino acid substitution at position 405 in a human IgG1 heavy chain, and a second CH3 region which has an amino acid substitution at a position selected from the group consisting of: 366, 368, 370, 399, 407 and 409 in a human IgG1 heavy chain. In one embodiment, the bispecific antibody of the invention as defined in any of the embodiments disclosed herein, comprises a first CH3 region which has an amino acid substitution at position 407 in a human IgG1 heavy chain, and a second CH3 region which has an amino acid substitution at a position selected from the group consisting of: 366, 368, 370, 399, 405, and 409 in a human IgG1 heavy chain. In one embodiment, the bispecific antibody of the invention as defined in any of the embodiments disclosed herein, comprises a first CH3 region which has an amino acid substitution at position 409 in a human IgG1 heavy chain, and a second CH3 region has an amino acid substitution at a position selected from the group consisting of: 366, 368, 370, 399, 405, and 407 in a human IgG1 heavy chain. Accordingly, in one embodiment the bispecific antibody of the invention as defined in any of the embodiments disclosed herein, comprises the sequences of said first and second CH3 regions containing asymmetrical mutations, i.e. mutations at different positions in the two CH3 regions, e.g. a mutation at position 405 in one of the CH3 regions and a mutation at position 409 in the other CH3 region. In one embodiment of the bispecific antibody of the invention as defined in any of the embodiments disclosed herein, the first CH3 region has an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region has an amino-acid substitution at a position selected from the group consisting of: 366, 368, 370, 399, 405 and 407. In one such embodiment, said first CH3 region has an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region has an amino acid other than Phe, e.g. Gly, Ala, Val, Ile, Ser, Thr, Met, Arg, His, Asp, Asn, Glu, Gln, Pro, Trp, Tyr, Cys, Lys, or Leu, at position 405. In a further embodiment hereof, said first CH3 region has an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region has an amino acid other than Phe, Arg or Gly, e.g. Leu, Ala, Val, Ile, Ser, Thr, Met, Lys, His, Asp, Asn, Glu, Gln, Pro, Trp, Tyr, or Cys, at position 405.

In another embodiment of the bispecific antibody of the invention as defined in any of the embodiments disclosed herein, said first CH3 region comprises a Phe at position 405 and an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region comprises an amino acid other than Phe, e.g. Gly, Ala, Val, Ile, Ser, Thr, Lys, Arg, His, Asp, Asn, Glu, Gln, Pro, Trp, Tyr, Leu, Met, or Cys, at position 405 and a Lys at position 409. In a further embodiment hereof, said first CH3 region comprises a Phe at position 405 and an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region comprises an amino acid other than Phe, Arg or Gly, e.g. Leu, Ala, Val, Ile, Ser, Thr, Met, Lys, His, Asp, Asn, Glu, Gln, Pro, Trp, Tyr, or Cys, at position 405 and a Lys at position 409.

In another embodiment of the bispecific antibody of the invention as defined in any of the embodiments disclosed herein, said first CH3 region comprises a Phe at position 405 and an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region comprises a Leu at position 405 and a Lys at position 409. In a further embodiment hereof, said first CH3 region comprises a Phe at position 405 and an Arg at position 409 and said second CH3 region comprises an amino acid other than Phe, Arg or Gly, e.g. Leu, Ala, Val, Ile, Ser, Thr, Lys, Met, His, Asp, Asn, Glu, Gln, Pro, Trp, Tyr, or Cys, at position 405 and a Lys at position 409. In another embodiment, said first CH3 region comprises Phe at position 405 and an Arg at position 409 and said second CH3 region comprises a Leu at position 405 and a Lys at position 409.

In a further embodiment of the bispecific antibody of the invention as defined in any of the embodiments disclosed herein, said first CH3 region comprises an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region comprises a Lys at position 409, a Thr at position 370 and a Leu at position 405. In a further embodiment, said first CH3 region comprises an Arg at position 409 and said second CH3 region comprises a Lys at position 409, a Thr at position 370 and a Leu at position 405.

In an even further embodiment of the bispecific antibody of the invention as defined in any of the embodiments disclosed herein, said first CH3 region comprises a Lys at position 370, a Phe at position 405 and an Arg at position 409 and said second CH3 region comprises a Lys at position 409, a Thr at position 370 and a Leu at position 405.

In another embodiment of the bispecific antibody of the invention as defined in any of the embodiments disclosed herein, said first CH3 region comprises an amino acid other than Lys, Leu or Met, e.g. Gly, Ala, Val, Ile, Ser, Thr, Phe, Arg, His, Asp, Asn, Glu, Gin, Pro, Trp, Tyr, or Cys, at position 409 and said second CH3 region comprises a Lys at position 409 and: a) an Ile at position 350 and a Leu at position 405, or b) a Thr at position 370 and a Leu at position 405.

In another embodiment of the bispecific antibody of the invention as defined in any of the embodiments disclosed herein, said first CH3 region comprises an Arg at position 409 and said second CH3 region comprises a Lys at position 409 and: a) an Ile at position 350 and a Leu at position 405, or b) a Thr at position 370 and a Leu at position 405.

In another embodiment of the bispecific antibody of the invention as defined in any of the embodiments disclosed herein, said first CH3 region comprises a Thr at position 350, a Lys at position 370, a Phe at position 405 and an Arg at position 409 and said second CH3 region comprises a Lys at position 409 and: a) an Ile at position 350 and a Leu at position 405, or b) a Thr at position 370 and a Leu at position 405.

In another embodiment of the bispecific antibody of the invention as defined in any of the embodiments disclosed herein, said first CH3 region comprises a Thr at position 350, a Lys at position 370, a Phe at position 405 and an Arg at position 409 and said second CH3 region comprises an Ile at position 350, a Thr at position 370, a Leu at position 405 and a Lys at position 409. In one embodiment of the bispecific antibody of the invention as defined in any of the embodiments disclosed herein, said first CH3 region has an amino acid other than Lys, Leu or Met at position 409 and said second CH3 region has an amino acid other than Phe at position 405, such as other than Phe, Arg or Gly at position 405; or said first CH3 region has an amino acid other than Lys, Leu or Met at position 409 and said second CH3 region has an amino acid other than Tyr, Asp, Glu, Phe, Lys, Gin, Arg, Ser or Thr at position 407.

In one embodiment, the bispecific antibody of the invention as defined in any of the embodiments disclosed herein comprises a first CH3 region having an amino acid other than Lys, Leu or Met at position 409 and a second CH3 region having an amino acid other than Tyr, Asp, Glu, Phe, Lys, Gin, Arg, Ser or Thr at position 407.

In one embodiment, the bispecific antibody of the invention as defined in any of the embodiments disclosed herein comprises a first CH3 region having a Tyr at position 407 and an amino acid other than Lys, Leu or Met at position 409 and a second CH3 region having an amino acid other than Tyr, Asp, Glu, Phe, Lys, Gin, Arg, Ser or Thr at position 407 and a Lys at position 409.

In one embodiment of invention, the bispecific antibody as defined in any of the embodiments disclosed herein comprises a first CH3 region having a Tyr at position 407 and an Arg at position 409 and a second CH3 region having an amino acid other than Tyr, Asp, Glu, Phe, Lys, Gln, Arg, Ser or Thr at position 407 and a Lys at position 409.

In another embodiment of invention, said first CH3 region has an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region has an amino acid other than Tyr, Asp, Glu, Phe, Lys, Gln, Arg, Ser or Thr, e.g. Leu, Met, Gly, Ala, Val, Ile, His, Asn, Pro, Trp, or Cys, at position 407. In another embodiment of invention, said first CH3 region has an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region has an Ala, Gly, His, Ile, Leu, Met, Asn, Val or Trp at position 407.

In another embodiment of the bispecific antibody of the invention as defined in any of the embodiments disclosed herein, said first CH3 region has an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region has a Gly, Leu, Met, Asn or Trp at position 407.

In another embodiment of the bispecific antibody of the invention as defined in any of the embodiments disclosed herein, said first CH3 region has a Tyr at position 407 and an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region has an amino acid other than Tyr, Asp, Glu, Phe, Lys, Gln, Arg, Ser or Thr, e.g. Leu, Met, Gly, Ala, Val, Ile, His, Asn, Pro, Trp, or Cys, at position 407 and a Lys at position 409. In another embodiment of the bispecific antibody of the invention as defined in any of the embodiments disclosed herein, said first CH3 region has a Tyr at position 407 and an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region has an Ala, Gly, His, Ile, Leu, Met, Asn, Val or Trp at position 407 and a Lys at position 409.

In another embodiment of the bispecific antibody of the invention as defined in any of the embodiments disclosed herein, said first CH3 region has a Tyr at position 407 and an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region has a Gly, Leu, Met, Asn or Trp at position 407 and a Lys at position 409.

In another embodiment of the bispecific antibody of the invention as defined in any of the embodiments disclosed herein, said first CH3 region has a Tyr at position 407 and an Arg at position 409 and said second CH3 region has an amino acid other than Tyr, Asp, Glu, Phe, Lys, Gin, Arg, Ser or Thr, e.g. Leu, Met, Gly, Ala, Val, Ile, His, Asn, Pro, Trp, or Cys, at position 407 and a Lys at position 409.

In another embodiment of the bispecific antibody of the invention as defined in any of the embodiments disclosed herein, said first CH3 region has a Tyr at position 407 and an Arg at position 409 and said second CH3 region has an Ala, Gly, His, Ile, Leu, Met, Asn, Val or Trp at position 407 and a Lys at position 409.

In another embodiment of the bispecific antibody of the invention as defined in any of the embodiments disclosed herein, said first CH3 region has a Tyr at position 407 and an Arg at position 409 and said second CH3 region has a Gly, Leu, Met, Asn or Trp at position 407 and a Lys at position 409.

In another embodiment of the bispecific antibody of the invention as defined in any of the embodiments disclosed herein, the first CH3 region has an amino acid other than Lys, Leu or Met, e.g. Gly, Ala, Val,

Ile, Ser, Thr, Phe, Arg, His, Asp, Asn, Glu, Gin, Pro, Trp, Tyr, or Cys, at position 409, and the second CH3 region has

(i) an amino acid other than Phe, Leu and Met, e.g. Gly, Ala, Val, Ile, Ser, Thr, Lys, Arg, His, Asp, Asn, Glu, Gin, Pro, Trp, Tyr, or Cys, at position 368, or

(ii) a Trp at position 370, or

(iii) an amino acid other than Asp, Cys, Pro, Glu or Gin, e.g. Phe, Leu, Met, Gly, Ala, Val, Ile, Ser, Thr, Lys, Arg, His, Asn, Trp, Tyr, or Cys, at position 399 or

(iv) an amino acid other than Lys, Arg, Ser, Thr, or Trp, e.g. Phe, Leu, Met, Ala, Val, Gly, Ile, Asn, His, Asp, Glu, Gin, Pro, Tyr, or Cys, at position 366.

In one embodiment, the first CH3 region has an Arg, Ala, His or Gly at position 409, and the second CH3 region has

(i) a Lys, Gin, Ala, Asp, Glu, Gly, His, Ile, Asn, Arg, Ser, Thr, Val, or Trp at position 368, or

(ii) a Trp at position 370, or

(iii) an Ala, Gly, Ile, Leu, Met, Asn, Ser, Thr, Trp, Phe, His, Lys, Arg or Tyr at position 399, or

(iv) an Ala, Asp, Glu, His, Asn, Val, Gin, Phe, Gly, Ile, Leu, Met, or Tyr at position 366.

In one embodiment, the first CH3 region has an Arg at position 409, and the second CH3 region has

(i) an Asp, Glu, Gly, Asn, Arg, Ser, Thr, Val, or Trp at position 368, or

(ii) a Trp at position 370, or

(iii) a Phe, His, Lys, Arg or Tyr at position 399, or

(iv) an Ala, Asp, Glu, His, Asn, Val, Gln at position 366.

In a preferred embodiment of the invention, the bispecific antibody comprises a first and second heavy chain, wherein each of said first and second heavy chains comprises at least a hinge region, a CH2 and a CH3 region, wherein (i) the amino acid in the position corresponding to F405 in human IgG1 heavy chain is L in said first heavy chain, and the amino acid in the position corresponding to K409 in human IgG1 heavy chain is R in said second heavy chain, or (ii) the amino acid in the position corresponding to K409 in human IgG1 heavy chain is R in said first heavy chain, and the amino acid in the position corresponding to F405 in human IgG1 heavy chain is L in said second heavy chain. In addition to the above-specified amino-acid substitutions, said first and second heavy chains may contain further amino-acid substitutions, deletion or insertions relative to wild-type heavy chain sequences.

In one embodiment of the invention, neither said first nor said second Fc sequence comprises a Cys-Pro-Ser-Cys sequence in the (core) hinge region.

In a further embodiment of the invention, both said first and said second Fc sequence comprise a Cys-Pro-Pro-Cys sequence in the (core) hinge region.

Traditional methods such as the hybrid hybridoma and chemical conjugation methods (Marvin and Zhu (2005) Acta Pharmacol Sin 26:649) can be used in the preparation of the bispecific antibodies of the invention. Co-expression in a host cell of two antibodies, consisting of different heavy and light chains, leads to a mixture of possible antibody products in addition to the desired bispecific antibody, which can then be isolated by, e.g., affinity chromatography or similar methods.

Strategies favoring the formation of a functional bispecific, product, upon co-expression of different antibody constructs can also be used, e.g., the method described by Lindhofer et al. (1995 J Immunol 155:219). Fusion of rat and mouse hybridomas producing different antibodies leads to a limited number of heterodimeric proteins because of preferential species-restricted heavy/light chain pairing. Another strategy to promote formation of heterodimers over homodimers is a “knob-into-hole” strategy in which a protuberance is introduced on a first heavy-chain polypeptide and a corresponding cavity in a second heavy-chain polypeptide, such that the protuberance can be positioned in the cavity at the interface of these two heavy chains so as to promote heterodimer formation and hinder homodimer formation. “Protuberances” are constructed by replacing small amino-acid side-chains from the interface of the first polypeptide with larger side chains. Compensatory “cavities” of identical or similar size to the protuberances are created in the interface of the second polypeptide by replacing large amino-acid side-chains with smaller ones (U.S. Pat. No. 5,731,168). EP1870459 (Chugai) and WO2009089004 (Amgen) describe other strategies for favoring heterodimer formation upon co-expression of different antibody domains in a host cell. In these methods, one or more residues that make up the CH3-CH3 interface in both CH3 domains are replaced with a charged amino acid such that homodimer formation is electrostatically unfavorable and heterodimerization is electrostatically favorable. WO2007110205 (Merck) describe yet another strategy, wherein differences between IgA and IgG CH3 domains are exploited to promote heterodimerization.

Another in vitro method for producing bispecific antibodies has been described in WO2008119353 (Genmab), wherein a bispecific antibody is formed by “Fab-arm” or “half-molecule” exchange (swapping of a heavy chain and attached light chain) between two monospecific IgG4- or IgG4-like antibodies upon incubation under reducing conditions. The resulting product is a bispecific antibody having two Fab arms which may comprise different sequences.

A preferred method for preparing bispecific PD-L1xCD137 antibodies of the present invention includes the methods described in WO2011131746 and WO2013060867 (Genmab) comprising the following steps:

a) providing a first antibody comprising an Fc region, said Fc region comprising a first CH3 region;

b) providing a second antibody comprising a second Fc region, said Fc region comprising a second CH3 region, wherein the first antibody is a CD137 antibody and the second antibody is a PD-L1 antibody, or vice versa;

wherein the sequences of said first and second CH3 regions are different and are such that the heterodimeric interaction between said first and second CH3 regions is stronger than each of the homodimeric interactions of said first and second CH3 regions;

c) incubating said first antibody together with said second antibody under reducing conditions; and

d) obtaining said bispecific PD-L1xCD137 antibody.

Similarly, there is provided a method for producing an antibody according to the invention, comprising the steps of:

a) culturing a host cell producing a first antibody comprising an antigen-binding region capable of binding to human CD137 as defined herein and purifying said first antibody from the culture;

b) culturing a host cell producing a second antibody comprising an antigen-binding region capable of binding to human PD-L1 as defined herein purifying said second antibody from the culture;

c) incubating said first antibody together with said second antibody under reducing conditions sufficient to allow the cysteines in the hinge region to undergo disulfide-bond isomerization, and

d) obtaining said bispecific antibody.

In one embodiment of the invention, the said first antibody together with said second antibody are incubated under reducing conditions sufficient to allow the cysteines in the hinge region to undergo disulfide-bond isomerization, wherein the heterodimeric interaction between said first and second antibodies in the resulting heterodimeric antibody is such that no Fab-arm exchange occurs at 0.5 mM GSH after 24 hours at 37° C.

Without being limited to theory, in step c), the heavy-chain disulfide bonds in the hinge regions of the parent antibodies are reduced and the resulting cysteines are then able to form inter heavy-chain disulfide bonds with cysteine residues of another parent antibody molecule (originally with a different specificity). In one embodiment of this method, the reducing conditions in step c) comprise the addition of a reducing agent, e.g. a reducing agent selected from the group consisting of: 2-mercaptoethylamine (2-MEA), dithiothreitol (DTT), dithioerythritol (DTE), glutathione, tris(2-carboxyethyl)phosphine (TCEP), L-cysteine and beta-mercapto-ethanol, preferably a reducing agent selected from the group consisting of: 2-mercaptoethylamine, dithiothreitol and tris(2-carboxyethyl)phosphine. In a further embodiment, step c) comprises restoring the conditions to become non-reducing or less reducing, for example by removal of a reducing agent, e.g. by desalting.

For this method, any of the CD137 and PD-L1 antibodies described above may be used including first and second CD137 and PD-L1 antibodies, respectively, comprising a first and/or second Fc region. Examples of such first and second Fc regions, including combination of such first and second Fc regions may include any of those described above. In a particular embodiment, the first and second CD137 and PD-L1 antibodies, respectively, may be chosen so as to obtain a bispecific antibody as described herein.

In one embodiment of this method, said first and/or second antibodies are full-length antibodies. The Fc regions of the first and second antibodies may be of any isotype, including, but not limited to, IgG1, IgG2, IgG3 or IgG4. In one embodiment of this method, the Fc regions of both said first and said second antibodies are of the IgG1 isotype. In another embodiment, one of the Fc regions of said antibodies is of the IgG1 isotype and the other of the IgG4 isotype. In the latter embodiment, the resulting bispecific antibody comprises an Fc sequence of an IgG1 and an Fc sequence of IgG4 and may thus have interesting intermediate properties with respect to activation of effector functions.

In a further embodiment, one of the antibody starting proteins has been engineered to not bind Protein A, thus allowing to separate the heterodimeric protein from said homodimeric starting protein by passing the product over a protein A column.

As described above, the sequences of the first and second CH3 regions of the homodimeric starting antibodies are different and are such that the heterodimeric interaction between said first and second CH3 regions is stronger than each of the homodimeric interactions of said first and second CH3 regions. More details on these interactions and how they can be achieved are provided in WO2011131746 and WO2013060867 (Genmab), which are hereby incorporated by reference in their entirety. In particular, a stable bispecific PD-L1xCD137 antibody can be obtained at high yield using the above method of the invention on the basis of two homodimeric starting antibodies which bind CD137 and PD-L1, respectively, and contain only a few, conservative, asymmetrical mutations in the CH3 regions. Asymmetrical mutations mean that the sequences of said first and second CH3 regions contain amino acid substitutions at non-identical positions.

The bispecific antibodies of the invention may also be obtained by co-expression of constructs encoding the first and second polypeptides in a single cell. Thus, in a further aspect, the invention relates to a method for producing a bispecific antibody, said method comprising the following steps:

a) providing a first nucleic-acid construct encoding a first polypeptide comprising a first Fc sequence and a first antigen-binding region of a first antibody heavy chain, said first Fc sequence comprising a first CH3 region,

b) providing a second nucleic-acid construct encoding a second polypeptide comprising a second Fc sequence and a second antigen-binding region of a second antibody heavy chain, said second Fc sequence comprising a second CH3 region,

wherein the sequences of said first and second CH3 regions are different and are such that the heterodimeric interaction between said first and second CH3 regions is stronger than each of the homodimeric interactions of said first and second CH3 regions, and wherein said first homodimeric protein has an amino acid other than Lys, Leu or Met at position 409 and said second homodimeric protein has an amino-acid substitution at a position selected from the group consisting of: 366, 368, 370, 399, 405 and 407,

optionally wherein said first and second nucleic acid constructs encode light chain sequences of said first and second antibodies

c) co-expressing said first and second nucleic-acid constructs in a host cell, and

d) obtaining said heterodimeric protein from the cell culture.

In some embodiments of the invention, the binding agent according to the present invention comprises, in addition to the antigen-binding regions, an Fc region consisting of the Fc sequences of the two heavy chains.

The first and second Fc sequences may each be of any isotype, including, but not limited to, IgG1, IgG2, IgG3 and IgG4, and may comprise one or more mutations or modifications. In one embodiment, each of the first and second Fc sequences is of the IgG4 isotype or derived therefrom, optionally with one or more mutations or modifications. In another embodiment, each of the first and second Fc sequences is of the IgG1 isotype or derived therefrom, optionally with one or more mutations or modifications. In another embodiment, one of the Fc sequences is of the IgG1 isotype and the other of the IgG4 isotype, or is derived from such respective isotypes, optionally with one or more mutations or modifications.

In one embodiment of the invention, one or both Fc sequences are effector-function-deficient. For example, the Fc sequence(s) may be of an IgG4 isotype, or a non-IgG4 type, e.g. IgG1, IgG2 or IgG3, which has been mutated such that the ability to mediate effector functions, such as ADCC, has been reduced or even eliminated. Such mutations have e.g. been described in Dall'Acqua WF et al., J Immunol. 177(2):1129-1138 (2006) and Hezareh M, J Virol.; 75(24):12161-12168 (2001). In another embodiment, one or both Fc sequences comprise an IgG1 wildtype sequence.

The term “effector functions” in the context of the present invention includes any functions mediated by components of the immune system that result, for example, in the killing of diseased cells such as tumor cells, or in the inhibition of tumor growth and/or inhibition of tumor development, including inhibition of tumor dissemination and metastasis. Preferably, the effector functions in the context of the present invention are T cell mediated effector functions. Such functions comprise ADCC, ADCP or CDC.

### Antibody-Dependent Cell-Mediated Cytotoxicity (ADCC)

Antibody-dependent cell-mediated cytotoxicity (ADCC) is the killing of an antibody-coated target cell by a cytotoxic effector cell through a nonphagocytic process, characterised by the release of the content of cytotoxic granules or by the expression of cell death-inducing molecules. ADCC is independent of the immune complement system that also lyses targets but does not require any other cell. ADCC is triggered through interaction of target-bound antibodies (belonging to IgG or IgA or IgE classes) with certain Fc receptors (FcRs), glycoproteins present on the effector cell surface that bind the Fc region of immunoglobulins (Ig). Effector cells that mediate ADCC include natural killer (NK) cells, monocytes, macrophages, neutrophils, eosinophils and dendritic cells. ADCC is a rapid effector mechanism whose efficacy is dependent on a number of parameters (density and stability of the antigen on the surface of the target cell; antibody affinity and FcR-binding affinity). ADCC involving human IgG1, the most used IgG subclass for therapeutic antibodies, is highly dependent on the glycosylation profile of its Fc portion and on the polymorphism of Fcy receptors.

### Antibody-Dependent Cellular Phagocytosis (ADCP)

ADCP is one crucial mechanism of action of many antibody therapies. It is defined as a highly regulated process by which antibodies eliminate bound targets via connecting its Fc domain to specific receptors on phagocytic cells, and eliciting phagocytosis. Unlike ADCC, ADCP can be mediated by monocytes, macrophages, neutrophils, and dendritic cells, through FcyRlla, FcyRl, and FcyRllla, of which FcyRlla (CD32a) on macrophages represent the predominant pathway.

### Complement-Dependent Cytotoxicity (CDC)

CDC is another cell-killing method that can be directed by antibodies. IgM is the most effective isotype for complement activation. IgG1 and IgG3 are also both very effective at directing CDC via the classical complement-activation pathway. Preferably, in this cascade, the formation of antigen-antibody complexes results in the uncloaking of multiple C1q binding sites in close proximity on the CH2 domains of participating antibody molecules such as IgG molecules (C1q is one of three subcomponents of complement C1). Preferably these uncloaked C1q binding sites convert the previously low-affinity C1g-IgG interaction to one of high avidity, which triggers a cascade of events involving a series of other complement proteins and leads to the proteolytic release of the effector-cell chemotactic/activating agents C3a and C5a. Preferably, the complement cascade ends in the formation of a membrane attack complex, which creates pores in the cell membrane that facilitate free passage of water and solutes into and out of the cell.

Antibodies according to the present invention may comprise modifications in the Fc region. When an antibody comprises such modifications, it may become an inert, or non-activating, antibody. The term “inertness”, “inert” or “non-activating” as used herein, refers to an Fc region which is at least not able to bind any Fcy receptors, induce Fc-mediated cross-linking of FcRs, or induce FcR-mediated cross-linking of target antigens via two Fc regions of individual antibodies, or is not able to bind C1q. The inertness of an Fc region of a humanized or chimeric CD137 or PD-L1 antibody is advantageously tested using the antibody in a monospecific format.

Several variants can be constructed to make the Fc region of an antibody inactive for interactions with Fcy (gamma) receptors and C1q for therapeutic antibody development. Examples of such variants are described herein.

Thus, in one embodiment of the antibody of the invention, said antibody comprises a first and a second heavy chain, wherein one or both heavy chains are modified so that the antibody induces Fc-mediated effector function to a lesser extent relative to an antibody which is identical, except for comprising non-modified first and second heavy chains. Said Fc-mediated effector function may be measured by determining, by binding to Fcy receptors, by binding to C1q, or by induction of Fc-mediated cross-linking of FcRs.

In another such embodiment, the heavy and light chain constant sequences have been modified so that binding of C1q to said antibody is reduced compared to an unmodified antibody by at least 70%, at least 80%, at least 90%, at least 95%, at least 97%, or 100%, wherein C1q binding is determined by ELISA. Thus, amino acids in the Fc region that play a dominant role in the interactions with C1q and the Fcy receptors may be modified.

Examples of amino acid positions that may be modified, e.g. in an IgG1 isotype antibody, include positions L234, L235 and P331. Combinations thereof, such as L234F/L235E/P331S, can cause a profound decrease in binding to human CD64, CD32, CD16 and C1q.

Hence, in one embodiment, the amino acid in at least one position corresponding to L234, L235 and P331, may be A, A and S, respectively (Xu et al., 2000, Cell Immunol. 200(1):16-26; Oganesyan et al., 2008, Acta Cryst. (D64):700-4). Also, L234F and L235E amino acid substitutions can result in Fc regions with abrogated interactions with Fcy receptors and C1q (Canfield et al., 1991, J. Exp. Med. (173):1483-91; Duncan et al., 1988, Nature (332):738-40). Hence, in one embodiment, the amino acids in the positions corresponding to L234 and L235, may be F and E, respectively. A D265A amino acid substitution can decrease binding to all Fcy receptors and prevent ADCC (Shields et al., 2001, J. Biol. Chem. (276):6591-604). Hence, in one embodiment, the amino acid in the position corresponding to D265 may be A. Binding to C1q can be abrogated by mutating positions D270, K322, P329, and P331. Mutating these positions to either D270A or K322A or P329A or P331A can make the antibody deficient in CDC activity (Idusogie EE, et al., 2000, J Immunol. 164: 4178-84). Hence, in one embodiment, the amino acids in at least one position corresponding to D270, K322, P329 and P331, may be A, A, A, and A, respectively. An alternative approach to minimize the interaction of the Fc region with Fcy receptors and C1q is by removal of the glycosylation site of an antibody. Mutating position N297 to e.g. Q, A, or E removes a glycosylation site which is critical for IgG-Fc gamma Receptor interactions. Hence, in one embodiment, the amino acid in a position corresponding to N297, may be G, Q, A or E (Leabman et al., 2013, MAbs; 5(6):896-903). Another alternative approach to minimize interaction of the Fc region with Fcy receptors may be obtained by the following mutations; P238A, A327Q, P329A or E233P/L234V/L235A/G236del (Shields et al., 2001, J. Biol. Chem. (276):6591-604).

Alternatively, human IgG2 and IgG4 subclasses are considered naturally compromised in their interactions with C1q and Fc gamma Receptors although interactions with Fcy receptors were reported (Parren et al., 1992, J. Clin Invest. 90: 1537-1546; Bruhns et al., 2009, Blood 113: 3716-3725). Mutations abrogating these residual interactions can be made in both isotypes, resulting in reduction of unwanted side-effects associated with FcR binding. For IgG2, these include L234A and G237A, and for IgG4, L235E. Hence, in one embodiment, the amino acid in a position corresponding to L234 and G237 in a human IgG2 heavy chain, may be A and A, respectively. In one embodiment, the amino acid in a position corresponding to L235 in a human IgG4 heavy chain, may be E.

Other approaches to further minimize the interaction with Fcy receptors and C1q in IgG2 antibodies include those described in W02011066501 and Lightle, S., et al., 2010, Protein Science (19):753-62. The hinge region of the antibody can also be of importance with respect to interactions with Fcy receptors and complement (Brekke et al., 2006, J Immunol 177:1129-1138; Dall'Acqua WF, et al., 2006, J Immunol 177:1129-1138). Accordingly, mutations in or deletion of the hinge region can influence effector functions of an antibody.

Thus, in one embodiment, the antibody comprises a first and a second immunoglobulin heavy chain, wherein in at least one of said first and second immunoglobulin heavy chains one or more amino acids in the positions corresponding to positions L234, L235, D265, N297, and P331 in a human IgG1 heavy chain, are not L, L, D, N, and P, respectively.

In one embodiment, in both the first and second heavy chains one or more amino acids in the position corresponding to positions L234, L235, D265, N297, and P331 in a human IgG1 heavy chain, are not L, L, D, N, and P, respectively.

In one embodiment of the invention, in both said first and second heavy chains the amino acid in the position corresponding to position D265 in a human IgG1 heavy chain, is not D.

Thus, in one embodiment of the invention, in both said first and second heavy chains the amino acid in the position corresponding to position D265 in a human IgG1 heavy chain are selected from the group consisting of: A and E.

In a further embodiment of the invention, in at least one of said first and second heavy chains the amino acids in the positions corresponding to positions L234 and L235 in a human IgG1 heavy chain, are not L and L, respectively.

In a particular embodiment of the invention, in at least one of said first and second heavy chains the amino acids in the positions corresponding to positions L234 and L235 in a human IgG1 heavy chain, are F and E, respectively.

In one embodiment of the invention, in both said first and second heavy chains the amino acids in the positions corresponding to positions L234 and L235 in a human IgG1 heavy chain, are F and E, respectively.

In a particular embodiment of the invention, in at least one of said first and second heavy chains the amino acids in the positions corresponding to positions L234, L235, and D265 in a human IgG1 heavy chain, are F, E, and A, respectively.

In a particularly preferred embodiment of the invention, in both said first and second heavy chains the amino acids in the positions corresponding to positions L234, L235, and D265 in a human IgG1 heavy chain, are F, E, and A, respectively.

In a further particularly preferred embodiment of the invention, the binding agent is a bispecific antibody comprising a first and second heavy chain, wherein the positions corresponding to positions L234 and L235 in a human IgG1 heavy chain according to EU numbering of both the first heavy chain and the second heavy chain are F and E, respectively, and wherein (i) the position corresponding to F405 in a human IgG1 heavy chain according to EU numbering of the first heavy chain is L, and the position corresponding to K409 in a human IgG1 heavy chain according to EU numbering of the second heavy chain is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

In a further particularly preferred embodiment of the invention, the binding agent is a bispecific antibody comprising a first and second heavy chain, wherein the positions corresponding to positions L234, L235, and D265 in a human IgG1 heavy chain according to EU numbering of both the first heavy chain and the second heavy chain are F, E, and A, respectively, and wherein (i) the position corresponding to F405 in a human IgG1 heavy chain according to EU numbering of the first heavy chain is L, and the position corresponding to K409 in a human IgG1 heavy chain according to EU numbering of the second heavy chain is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

Antibody variants having the combination of three amino acid substitutions L234F, L235E and D265A and in addition the K409R or the F405L mutation are herein termed with the suffix “FEAR” or “FEAL”, respectively.

In a preferred embodiment, the bispecific antibody of the invention comprises:

(i) a half-molecule antibody derived from IgG1-CD137-FEAL, and a half-molecule antibody derived from IgG1-PDL1-547-FEAR, or

(ii) a half-molecule antibody derived from IgG1-CD137-FEAR, and a half-molecule antibody derived from and a half-molecule antibody derived from IgG1-PD-L1-547-FEAL.

In a further embodiment of the invention, one or both antibodies forming part of the bispecific antibody have been engineered to reduce or increase the binding to the neonatal Fc receptor (FcRn) in order to manipulate the serum half-life of the bispecific antibody. Techniques for increasing or reducing the serum half-life are well-known in the art. See for example Dall'Acqua et al. 2006, J. Biol. Chem., 281:23514-24; Hinton et al. 2006, J. Immunol., 176:346-56; and Zalevsky et al. 2010 Nat. Biotechnol., 28:157-9.

In a further embodiment, the binding agents or antibodies described herein are linked or conjugated to one or more therapeutic moieties, such as a cytokine, an immune-suppressant, an immune-stimulatory molecule and/or a radioisotope. Such conjugates are referred to herein as “immunoconjugates” or “drug conjugates”. Immunoconjugates which include one or more cytotoxins are referred to as “immunotoxins”.

In one embodiment, the first and/or second Fc sequence is conjugated to a drug or a prodrug or contains an acceptor group for the same. Such acceptor group may e.g. be an unnatural amino acid.

### RNA Delivery

It is particularly preferred according to the invention that the peptides, proteins or polypeptides described herein, in particular the peptide or protein antigens and/or antibodies, are administered in the form of RNA encoding the peptides, proteins or polypeptides described herein. In one embodiment, different peptides, proteins or polypeptides described herein are encoded by different RNA molecules.

In one embodiment, the RNA is formulated in a delivery vehicle. In one embodiment, the delivery vehicle comprises particles. In one embodiment, the delivery vehicle comprises at least one lipid. In one embodiment, the at least one lipid comprises at least one cationic lipid. In one embodiment, the lipid forms a complex with and/or encapsulates the RNA. In one embodiment, the lipid is comprised in a vesicle encapsulating the RNA. In one embodiment, the RNA is formulated in liposomes.

According to the disclosure, after administration of the RNA described herein, at least a portion of the RNA is delivered to a target cell. In one embodiment, at least a portion of the RNA is delivered to the cytosol of the target cell. In one embodiment, the RNA is translated by the target cell to produce the encoded peptide or protein.

Some aspects of the disclosure involve the targeted delivery of the RNA disclosed herein (e.g., RNA encoding a peptide or protein comprising an epitope) to certain tissues.

In one embodiment, the disclosure involves targeting the lymphatic system, in particular secondary lymphoid organs, more specifically spleen. Targeting the lymphatic system, in particular secondary lymphoid organs, more specifically spleen is in particular preferred if the RNA administered is RNA encoding a peptide or protein comprising an epitope.

In one embodiment, the target cell is a spleen cell. In one embodiment, the target cell is an antigen presenting cell such as a professional antigen presenting cell in the spleen. In one embodiment, the target cell is a dendritic cell in the spleen.

The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naive lymphocytes and initiate an adaptive immune response.

RNA may be delivered to spleen by so-called lipoplex formulations, in which the RNA is bound to liposomes comprising a cationic lipid and optionally an additional or helper lipid to form injectable nanoparticle formulations. The liposomes may be obtained by injecting a solution of the lipids in ethanol into water or a suitable aqueous phase. RNA lipoplex particles may be prepared by mixing the liposomes with RNA. Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

In the context of the present disclosure, the term “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

As used herein, a “cationic lipid” refers to a lipid having a net positive charge. Cationic lipids bind negatively charged RNA by electrostatic interaction to the lipid matrix. Generally, cationic lipids possess a lipophilic moiety, such as a sterol, an acyl or diacyl chain, and the head group of the lipid typically carries the positive charge. Examples of cationic lipids include, but are not limited to 1,2-di-O-octadecenyl-3-trimethylammonium propane (DOTMA), dimethyldioctadecylammonium (DDAB); 1,2-dioleoyl-3-trimethylammonium propane (DOTAP); 1,2-dioleoyl-3-dimethylammonium-propane (DODAP); 1,2-diacyloxy-3-dimethylammonium propanes; 1,2-dialkyloxy-3- dimethylammonium propanes; dioctadecyldimethyl ammonium chloride (DODAC), 2,3-di(tetradecoxy)propyl-(2-hydroxyethyl)-dimethylazanium (DMRIE), 1,2-dimyristoyl-sn-glycero-3-ethylphosphocholine (DM EPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy- N-[2(spermine carboxamide)ethyl]-N,N-dimethyl-l-propanamium trifluoroacetate (DOSPA). Preferred are DOTMA, DOTAP, DODAC, and DOSPA. In specific embodiments, the cationic lipid is DOTMA and/or DOTAP.

An additional lipid may be incorporated to adjust the overall positive to negative charge ratio and physical stability of the RNA lipoplex particles. In certain embodiments, the additional lipid is a neutral lipid. As used herein, a “neutral lipid” refers to a lipid having a net charge of zero. Examples of neutral lipids include, but are not limited to, 1,2-di-(9Z-octadecenoyI)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, cholesterol, and cerebroside. In specific embodiments, the additional lipid is DOPE, cholesterol and/or DOPC.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

The electric charge of the RNA lipoplex particles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol)) * (the total number of positive charges in the cationic lipid)]/[(RNA concentration (mol)) * (the total number of negative charges in RNA)].

The spleen targeting RNA lipoplex particles described herein at physiological pH preferably have a net negative charge such as a charge ratio of positive charges to negative charges from about 1.9:2 to about 1:2. In specific embodiments, the charge ratio of positive charges to negative charges in the RNA lipoplex particles at physiological pH is about 1.9:2.0, about 1.8:2.0, about 1.7:2.0, about 1.6:2.0, about 1.5:2.0, about 1.4:2.0, about 1.3:2.0, about 1.2:2.0, about 1.1:2.0, or about 1:2.0.

RNA delivery systems have an inherent preference to the liver. This pertains to lipid-based particles, cationic and neutral nanoparticles, in particular lipid nanoparticles such as liposomes, nanomicelles and lipophilic ligands in bioconjugates. Liver accumulation is caused by the discontinuous nature of the hepatic vasculature or the lipid metabolism (liposomes and lipid or cholesterol conjugates).

For in vivo delivery of RNA to the liver, a drug delivery system may be used to transport the RNA into the liver by preventing its degradation. For example, polyplex nanomicelles consisting of a poly(ethylene glycol) (PEG)-coated surface and an mRNA-containing core is a useful system because the nanomicelles provide excellent in vivo stability of the RNA, under physiological conditions. Furthermore, the stealth property provided by the polyplex nanomicelle surface, composed of dense PEG palisades, effectively evades host immune defenses.

### Pharmaceutical Composition

The agents described herein may be administered in pharmaceutical compositions or medicaments for therapeutic or prophylactic treatments and may be administered in the form of any suitable pharmaceutical composition.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation.

The pharmaceutical compositions of the present disclosure preferably comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cyctokines, such as monokines, lymphokines, interleukins, chemokines. The chemokines may be IL1, IL2, IL3, IL4, IL5, IL6, IL7, IL8, IL9, IL10, IL12, IFNa, IFNγ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys.

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” or “therapeutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In one embodiment, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In one embodiment, pharmaceutical compositions described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical compositions is formulated for systemic administration. In another preferred embodiment, the systemic administration is by intravenous administration.

The term “co-administering” as used herein means a process whereby different compounds or compositions (e.g., RNA encoding a peptide or protein comprising an epitope and a binding agent) are administered to the same patient. The RNA encoding a peptide or protein comprising an epitope and the binding agent may be administered simultaneously, at essentially the same time, or sequentially. If administration takes place sequentially, the binding agent may be administered before or after administration of the RNA encoding a peptide or protein comprising an epitope. Preferably, the binding agent is administered after administration of the RNA encoding a peptide or protein comprising an epitope. If administration takes place simultaneously the binding agent and the RNA encoding a peptide or protein comprising an epitope need not be administered within the same composition. The binding agent and the RNA encoding a peptide or protein comprising an epitope may be administered one or more times and the number of administrations of each component may be the same or different. In addition, the binding agent and the RNA encoding a peptide or protein comprising an epitope need not be administered at the same site.

### Uses

The agents described herein may be used in the therapeutic or prophylactic treatment of various diseases, in particular diseases in which provision of a peptide or protein comprising an epitope for inducing an immune response against an antigen in a subject to said subject results in a therapeutic or prophylactic effect. For example, provision of an antigen or epitope which is derived from a virus may be useful in the treatment of a viral disease caused by said virus. Provision of a tumor antigen or epitope may be useful in the treatment of a cancer disease wherein cancer cells express said tumor antigen.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment”, “treating” or “therapeutic intervention” relates to the management and care of a subject for the purpose of combating a condition such as a disease or disorder.

The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g. mouse, rat, rabbit, dog, cat, cattle, swine, sheep, horse or primate) that can be afflicted with or is susceptible to a disease or disorder (e.g., cancer) but may or may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In one embodiment of the disclosure, the aim is to provide an immune response against diseased cells expressing an antigen such as cancer cells expressing a tumor antigen, and to treat a disease such as a cancer disease involving cells expressing an antigen such as a tumor antigen.

A pharmaceutical composition comprising a peptide or protein comprising an epitope or a polynucleotide such as RNA encoding a peptide or protein comprising an epitope may be administered to a subject to elicit an immune response against an antigen comprising said epitope in the subject which may be therapeutic or partially or fully protective. A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated. Accordingly, pharmaceutical compositions described herein are applicable for inducing or enhancing an immune response. Pharmaceutical compositions described herein are thus useful in a prophylactic and/or therapeutic treatment of a disease involving an antigen or epitope.

As used herein, “immune response” refers to an integrated bodily response to an antigen or a cell expressing an antigen and refers to a cellular immune response and/or a humoral immune response. A cellular immune response includes, without limitation, a cellular response directed to cells expressing an antigen and being characterized by presentation of an antigen with class I or class II MHC molecule. The cellular response relates to T lymphocytes, which may be classified as helper T cells (also termed CD4+ T cells) that play a central role by regulating the immune response or killer cells (also termed cytotoxic T cells, CD8+ T cells, or CTLs) that induce apoptosis in infected cells or cancer cells. In one embodiment, administering a pharmaceutical composition of the present disclosure involves stimulation of an anti-tumor CD8+ T cell response against cancer cells expressing one or more tumor antigens. In as specific embodiment, the tumor antigens are presented with class I MHC molecule.

The present disclosure contemplates an immune response that may be protective, preventive, prophylactic and/or therapeutic. As used herein, “induces [or inducing] an immune response” may indicate that no immune response against a particular antigen was present before induction or it may indicate that there was a basal level of immune response against a particular antigen before induction, which was enhanced after induction. Therefore, “induces [or inducing] an immune response” includes “enhances [or enhancing] an immune response”.

The term “immunotherapy” relates to the treatment of a disease or condition by inducing, or enhancing an immune response. The term “immunotherapy” includes antigen immunization or antigen vaccination.

The terms “immunization” or “vaccination” describe the process of administering an antigen to an individual with the purpose of inducing an immune response, for example, for therapeutic or prophylactic reasons.

In one embodiment, the present disclosure envisions embodiments wherein RNA formulations such as RNA lipoplex particles as described herein targeting spleen tissue are administered. The RNA encodes, for example, a peptide or protein comprising an epitope as described, for example, herein. The RNA is taken up by antigen-presenting cells in the spleen such as dendritic cells to express the peptide or protein. Following optional processing and presentation by the antigen-presenting cells an immune response may be generated against the epitope resulting in a prophylactic and/or therapeutic treatment of a disease involving the epitope or an antigen comprising the epitope. In one embodiment, the immune response induced by the RNA described herein comprises presentation of an antigen or fragment thereof, such as an epitope, by antigen presenting cells, such as dendritic cells and/or macrophages, and activation of cytotoxic T cells due to this presentation. For example, peptides or proteins encoded by the RNAs or procession products thereof may be presented by major histocompatibility complex (MHC) proteins expressed on antigen presenting cells. The MHC peptide complex can then be recognized by immune cells such as T cells or B cells leading to their activation.

Accordingly, the present disclosure relates to RNA as described herein for use in a prophylactic and/or therapeutic treatment of a disease involving an antigen, preferably a cancer disease.

The term “macrophage” refers to a subgroup of phagocytic cells produced by the differentiation of monocytes. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B cell surface, resulting in T and B cell activation and further stimulation of the immune response. Macrophages belong to the class of antigen presenting cells. In one embodiment, the macrophages are splenic macrophages.

The term “dendritic cell” (DC) refers to another subtype of phagocytic cells belonging to the class of antigen presenting cells. In one embodiment, dendritic cells are derived from hematopoietic bone marrow progenitor cells. These progenitor cells initially transform into immature dendritic cells. These immature cells are characterized by high phagocytic activity and low T cell activation potential. Immature dendritic cells constantly sample the surrounding environment for pathogens such as viruses and bacteria. Once they have come into contact with a presentable antigen, they become activated into mature dendritic cells and begin to migrate to the spleen or to the lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T cell activation such as CD80, CD86, and CD40 greatly enhancing their ability to activate T cells. They also upregulate CCR7, a chemotactic receptor that induces the dendritic cell to travel through the blood stream to the spleen or through the lymphatic system to a lymph node. Here they act as antigen-presenting cells and activate helper T cells and killer T cells as well as B cells by presenting them antigens, alongside non-antigen specific co-stimulatory signals. Thus, dendritic cells can actively induce a T cell- or B cell-related immune response. In one embodiment, the dendritic cells are splenic dendritic cells.

The term “antigen presenting cell” (APC) is a cell of a variety of cells capable of displaying, acquiring, and/or presenting at least one antigen or antigenic fragment on (or at) its cell surface. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naive T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, such as antigen presenting cells to specific T cells.

The term “disease involving an antigen” or “disease involving an epitope” refers to any disease which implicates an antigen or epitope, e.g. a disease which is characterized by the presence of an antigen or epitope. The disease involving an antigen or epitope can be an infectious disease, or a cancer disease or simply cancer. As mentioned above, the antigen may be a disease-associated antigen, such as a tumor-associated antigen, a viral antigen, or a bacterial antigen and the epitope may be derived from such antigen.

The term “infectious disease” refers to any disease which can be transmitted from individual to individual or from organism to organism, and is caused by a microbial agent (e.g. common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g. chlamydia or gonorrhea), tuberculosis, HIV/acquired immune deficiency syndrome (AIDS), diphtheria, hepatitis B, hepatitis C, cholera, severe acute respiratory syndrome (SARS), the bird flu, and influenza.

The terms “cancer disease” or “cancer” refer to or describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. The term “cancer” according to the disclosure also comprises cancer metastases.

Combination strategies in cancer treatment may be desirable due to a resulting synergistic effect, which may be considerably stronger than the impact of a monotherapeutic approach. In one embodiment, the pharmaceutical composition is administered with an immunotherapeutic agent. As used herein “immunotherapeutic agent” relates to any agent that may be involved in activating a specific immune response and/or immune effector function(s). The present disclosure contemplates the use of an antibody as an immunotherapeutic agent. Without wishing to be bound by theory, antibodies are capable of achieving a therapeutic effect against cancer cells through various mechanisms, including inducing apoptosis, block components of signal transduction pathways or inhibiting proliferation of tumor cells. In certain embodiments, the antibody is a monoclonal antibody. A monoclonal antibody may induce cell death via antibody-dependent cell mediated cytotoxicity (ADCC), or bind complement proteins, leading to direct cell toxicity, known as complement dependent cytotoxicity (CDC). Non-limiting examples of anti-cancer antibodies and potential antibody targets (in brackets) which may be used in combination with the present disclosure include: Abagovomab (CA-125), Abciximab (CD41), Adecatumumab (EpCAM), Afutuzumab (CD20), Alacizumab pegol (VEGFR2), Altumomab pentetate (CEA), Amatuximab (MORAb-009), Anatumomab mafenatox (TAG-72), Apolizumab (HLA-DR), Arcitumomab (CEA), Atezolizumab (PD-L1), Bavituximab (phosphatidylserine), Bectumomab (CD22), Belimumab (BAFF), Bevacizumab (VEGF-A), Bivatuzumab mertansine (CD44 v6), Blinatumomab (CD 19), Brentuximab vedotin (CD30 TNFRSF8), Cantuzumab mertansin (mucin CanAg), Cantuzumab ravtansine (MUC1), Capromab pendetide (prostatic carcinoma cells), Carlumab (CNT0888), Catumaxomab (EpCAM, CD3), Cetuximab (EGFR), Citatuzumab bogatox (EpCAM), Cixutumumab (IGF-1 receptor), Claudiximab (Claudin), Clivatuzumab tetraxetan (MUC1), Conatumumab (TRAIL-R2), Dacetuzumab (CD40), Dalotuzumab (insulin-like growth factor I receptor), Denosumab (RANKL), Detumomab (B-lymphoma cell), Drozitumab (DR5), Ecromeximab (GD3 ganglioside), Edrecolomab (EpCAM), Elotuzumab (SLAMF7), Enavatuzumab (PDL192), Ensituximab (NPC-1C), Epratuzumab (CD22), Ertumaxomab (HER2/neu, CD3), Etaracizumab (integrin av133), Farletuzumab (folate receptor 1), FBTA05 (CD20), Ficlatuzumab (SCH 900105), Figitumumab (IGF-1 receptor), Flanvotumab (glycoprotein 75), Fresolimumab (TGF-β), Galiximab (CD80), Ganitumab (IGF-I), Gemtuzumab ozogamicin (CD33), Gevokizumab (ILIIβ), Girentuximab (carbonic anhydrase 9 (CA-IX)), Glembatumumab vedotin (GPNMB), Ibritumomab tiuxetan (CD20), Icrucumab (VEGFR-1), Igovoma (CA-125), Indatuximab ravtansine (SDC1), Intetumumab (CD51), Inotuzumab ozogamicin (CD22), Ipilimumab (CD 152), Iratumumab (CD30), Labetuzumab (CEA), Lexatumumab (TRAIL-R2), Libivirumab (hepatitis B surface antigen), Lintuzumab (CD33), Lorvotuzumab mertansine (CD56), Lucatumumab (CD40), Lumiliximab (CD23), Mapatumumab (TRAIL-R1), Matuzumab (EGFR), Mepolizumab (IL5), Milatuzumab (CD74), Mitumomab (GD3 ganglioside), Mogamulizumab (CCR4), Moxetumomab pasudotox (CD22), Nacolomab tafenatox (C242 antigen), Naptumomab estafenatox (5T4), Namatumab (RON), Necitumumab (EGFR), Nimotuzumab (EGFR), Nivolumab (IgG4), Ofatumumab (CD20), Olaratumab (PDGF-R a), Onartuzumab (human scatter factor receptor kinase), Oportuzumab monatox (EpCAM), Oregovomab (CA-125), Oxelumab (OX-40), Panitumumab (EGFR), Patritumab (HER3), Pemtumoma (MUC1), Pertuzuma (HER2/neu), Pintumomab (adenocarcinoma antigen), Pritumumab (vimentin), Racotumomab (N-glycolylneuraminic acid), Radretumab (fibronectin extra domain-B), Rafivirumab (rabies virus glycoprotein), Ramucirumab (VEGFR2), Rilotumumab (HGF), Rituximab (CD20), Robatumumab (IGF-1 receptor), Samalizumab (CD200), Sibrotuzumab (FAP), Siltuximab (IL6), Tabalumab (BAFF), Tacatuzumab tetraxetan (alpha-fetoprotein), Taplitumomab paptox (CD 19), Tenatumomab (tenascin C), Teprotumumab (CD221), Ticilimumab (CTLA- 4), Tigatuzumab (TRAIL-R2), TNX-650 (IL13), Tositumomab (CD20), Trastuzumab (HER2/neu), TRBS07 (GD2), Tremelimumab (CTLA-4), Tucotuzumab celmoleukin (EpCAM), Ublituximab (MS4A1), Urelumab (4-1 BB), Volociximab (integrin a581), Votumumab (tumor antigen CTAA 16.88), Zalutumumab (EGFR), and Zanolimumab (CD4).

Sequences

| SEQ | | |
|---|---|---|
| ID | NAME | SEQUENCE |
| 1 | VH_CD137-009 | QSLEESGGRLVTPGTPLTLTCTVSGFSLNDYWMSWVRQAPGKGL |
| | | EWIGYIDVGGSLYYASWAKGRFTISRTSTTVDLKMTSLTTEDTATY |
| | | FCARGGLTYGFDLWGPGTLVTVSS |
| 2 | VH_CD137-009_CDR1 | GFSLNDYW |
| 3 | VH_CD137-009_CDR2 | IDVGGSL |
| 4 | VH_CD137-009_CDR3 | ARGGLTYGFDL |
| 5 | VL_CD137-009 | DIVMTQTPASVSEPVGGTVTINCQASEDISSYLAWYQQKPGQRPK |
| | | RLIYGASDLASGVPSRFSASGSGTEYALTISDLESADAATYYCHYY |
| | | ATISGLGVAFGGGTEVVVK |
| 6 | VL_CD137-009_CDR1 | EDISSY |
| | VL_CD137-009_CDR2 | GAS |
| 7 | VL_CD137-009_CDR3 | HYYATISGLGVA |
| 8 | VH_CD137-009-H7 | EVQLVESGGGLVQPGRSLRLSCTASGFSLNDYWMSWVRQAPGK |
| | | GLEWVGYIDVGGSLYYAASVKGRFTISRDDSKSIAYLQMNSLKTED |
| | | TAVYYCARGGLTYGFDLWGQGTLVTVSS |
| 2 | VH_CD137-009- | GFSLNDYW |
| | H7_CDR1 | |
| 3 | VH_CD137-009- | IDVGGSL |
| | H7_CDR2 | |
| 4 | VH_CD137-009- | ARGGLTYGFDL |
| | H7_CDR3 | |
| 9 | VL_CD137-009-L2 | DIVMTQSPSSLSASVGDRVTITCQASEDISSYLAWYQQKPGKAPK |
| | | RLIYGASDLASGVPSRFSASGSGTDYTFTISSLQPEDIATYYCHYYA |
| | | TISGLGVAFGGGTKVEIK |
| 6 | VL_CD137-009-L2_CDR1 | EDISSY |
| | VL_CD137-009-L2_CDR2 | GAS |
| 7 | VL_CD137-009-L2_CDR3 | HYYATISGLGVA |
| 10 | VH-PD-L1-547 | EVQLLEPGGGLVQPGGSLRLSCEASGSTFSTYAMSWVRQAPGKG |
| | | LEWVSGFSGSGGFTFYADSVRGRFTISRDSSKNTLFLQMSSLRAE |
| | | DTAVYYCAIPARGYNYGSFQHWGQGTLVTVSS |
| 11 | VH-PD-L1-547-CDR1 | GSTFSTYA |
| 12 | VH-PD-L1-547-CDR2 | FSGSGGFT |
| 13 | VH-PD-L1-547-CDR3 | AIPARGYNYGSFQH |
| 14 | VL-PD-L1-547 | SYVLTQPPSVSVAPGQTARITCGGNNIGSKSVHWYQQKPGQAPVL |
| | | VVYDDNDRPSGLPERFSGSNSGNTATLTISRVEAGDEADYYCQV |
| | | WDSSSDHVVFGGGTKLTVL |
| 15 | VL-PD-L1-547-CDR1 | NIGSKS |
| | VL-PD-L1-547-CDR2 | DDN |
| 16 | VL-PD-L1-547-CDR3 | QVWDSSSDHVV |
| 17 | IgG1-FEAR-Fc | ASTKGPSVFPLAPSSKSTSGGTAALGCLVKDYFPEPVTVSWNSGA |
| | | LTSGVHTFPAVLQSSGLYSLSSVVTVPSSSLGTQTYICNVNHKPSN |
| | | TKVDKRVEPKSCDKTHTCPPCPAPEFEGGPSVFLFPPKPKDTLMIS |
| | | RTPEVTCVVVAVSHEDPEVKFNWYVDGVEVHNAKTKPREEQYNS |
| | | TYRVVSVLTVLHQDWLNGKEYKCKVSNKALPAPIEKTISKAKGQPR |
| | | EPQVYTLPPSREEMTKNQVSLTCLVKGFYPSDIAVEWESNGQPEN |
| | | NYKTTPPVLDSDGSFFLYSRLTVDKSRWQQGNVFSCSVMHEALH |
| | | NHYTQKSLSLSPGK |
| 18 | IgG1-FEAL-Fc | ASTKGPSVFPLAPSSKSTSGGTAALGCLVKDYFPEPVTVSWNSGA |
| | | LTSGVHTFPAVLQSSGLYSLSSVVTVPSSSLGTQTYICNVNHKPSN |
| | | TKVDKRVEPKSCDKTHTCPPCPAPEFEGGPSVFLFPPKPKDTLMIS |
| | | RTPEVTCVVVAVSHEDPEVKFNWYVDGVEVHNAKTKPREEQYNS |
| | | TYRVVSVLTVLHQDWLNGKEYKCKVSNKALPAPIEKTISKAKGQPR |
| | | EPQVYTLPPSREEMTKNQVSLTCLVKGFYPSDIAVEWESNGQPEN |
| | | NYKTTPPVLDSDGSFLLYSKLTVDKSRWQQGNVFSCSVMHEALH |
| | | NHYTQKSLSLSPGK |
| 19 | Kappa-C | RTVAAPSVFIFPPSDEQLKSGTASVVCLLNNFYPREAKVQWKVDN |
| | | ALQSGNSQESVTEQDSKDSTYSLSSTLTLSKADYEKHKVYACEVT |
| | | HQGLSSPVTKSFNRGEC |
| 20 | Lambda-C | GQPKAAPSVTLFPPSSEELQANKATLVCLISDFYPGAVTVAWKADS |
| | | SPVKAGVETTTPSKQSNNKYAASSYLSLTPEQWKSHRSYSCQVT |
| | | HEGSTVEKTVAPTECS |
| 21 | Human PD-L1 | MRIFAVFIFMTYWHLLNAFTVTVPKDLYVVEYGSNMTIECKFPVEK |
| | | QLDLAALIVYWEMEDKNIIQFVHGEEDLKVQHSSYRQRARLLKDQL |
| | | SLGNAALQITDVKLQDAGVYRCMISYGGADYKRITVKVNAPYNKIN |
| | | QRILVVDPVTSEHELTCQAEGYPKAEVIWTSSDHQVLSGKTTTTNS |
| | | KREEKLFNVTSTLRINTTTNEIFYCTFRRLDPEENHTAELVIPELP |
| | | LAHPPNERTHLVILGAILLCLGVALTFIFRLRKGRMMDVKKCGIQD |
| | | TNSKKQSDTHLEET |
| 22 | Human CD137 | MGNSCYNIVATLLLVLNFERTRSLQDPCSNCPAGTFCDNNRNQIC |
| | | SPCPPNSFSSAGGQRTCDICRQCKGVFRTRKECSSTSNAECDCT |
| | | PGFHCLGAGCSMCEQDCKQGQELTKKGCKDCCFGTFNDQKRGI |
| | | CRPWTNCSLDGKSVLVNGTKERDVVCGPSPADLSPGASSVTPPA |
| | | PAREPGHSPQIISFFLALTSTALLFLLFFLTLRFSVVKRGRKKL |
| | | QLYIFKPFMRPVQTTQEEDGCSCRFPEEEEGGCEL |
| 23 | Human CD137 (mature | LQDPCSNCPAGTFCDNNRNQICSPCPPNSFSSAGGQRTCDICRQ |
| | protein) | CKGVFRTRKECSSTSNAECDCTPGFHCLGAGCSMCEQDCKQGQ |
| | | ELTKKGCKDCCFGTFNDQKRGICRPWTNCSLDGKSVLVNGTKERD |
| | | VVCGPSPADLSPGASSVTPPAPAREPGHSPQIISFFLALTSTALLF |
| | | LLFFLTLRFSVVKRGRKKLLYIFKQPFMRPVQTTQEEDGCSCRFPE |
| | | EEEGGCEL |
| 24 | VH-CD137-005-FEAR | QSVEESGGRLVTPGTPLTLTCTASGFTISDFHVTWVRQAPGKGLE |
| | | WIGTIITSASTTAYATWARGRFTISKSSTTVNLKIVSPTTEDTAT |
| | | YFCARSTYTDTSGYYFDFWGQGTLVTVSS |
| 25 | VH-CD137-005- | GFTISDFH |
| | FEAR_CDR1 | |
| 26 | VH-CD137-005- | IITSASTT |
| | FEAR_CDR2 | |
| 27 | VH-CD137-005- | ARSTYTDTSGYYFDF |
| | FEAR_CDR3 | |
| 28 | VL-CD137-005 (Kappa) | AQVLTQTASPVSAAVGGTVIINCQSSQSIYNGNRLSWYQQKPGQP |
| | | PKLLIYSASTLASGVSSRFKGSGSGTQFTLAISDVQSDDAATYYCL |
| | | GSYDCDSADCFAFGGGTEVVVE |
| 29 | VL-CD137-005_CDR1 | QSIYNGNR |
| | VL-CD137-005_CDR2 | SAS |
| 30 | VL-CD137-005_CDR3 | LGSYDCDSADCFA |

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1

### A Mouse-Reactive PD-L1x4-1BB Bispecific Antibody Amplifies Therapeutic RNA-Vaccine Induced Antigen-Specific T-Cell Responses

In order to assess the effect of a mouse-reactive PD-L1x4-1BB bispecific antibody (bsAb) on RNA-vaccine induced T-cell responses in vivo, C57BL/6 mice (n=5 per group) were vaccinated intravenously (i.v.) with 20 pg liposomal formulated TRP1 RNA (TRP1 RNA-LPX) on day 1 and subsequently dosed intraperitoneally (i.p.) with either 50 μg, 10 μg or 2 μg of mouse-reactive PD-L1x4-1BB bsAb per mouse on days 2 and 5. The control group received the TRP1 RNA-vaccine in combination with 50 μg of isotype control antibody. TRP1 is the murine melanosomal antigen tyrosine-related protein-1 and is a self-antigen expressed constitutively on B16-F10 melanoma cells as well as in normal melanocytes. Anti-tumor efficacy of this TRP1 vaccine was previously demonstrated in Kranz, L. M. et al. Nature 534, 396-401 (2016). Lymphocyte subsets and TRP1-specific CD8+ T-cell responses in peripheral blood were determined on day 8 via flow cytometry (BD FACSCelesta™, BD Biosciences). The extracellular staining was performed as described in Kranz, L. M. et al. Nature 534, 396-401 (2016).

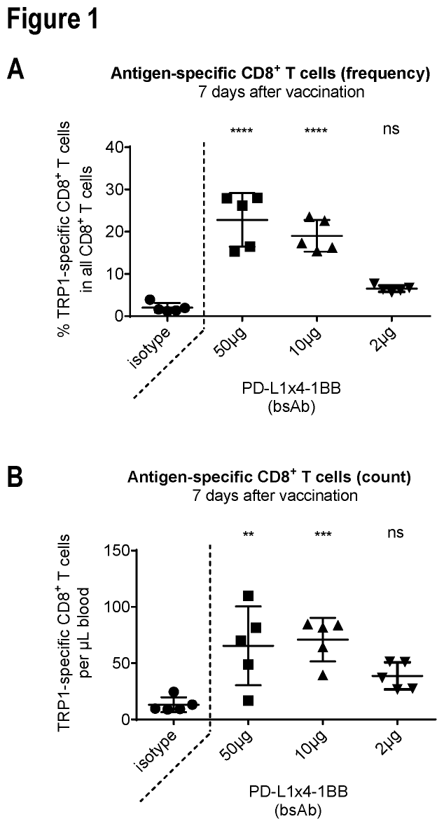

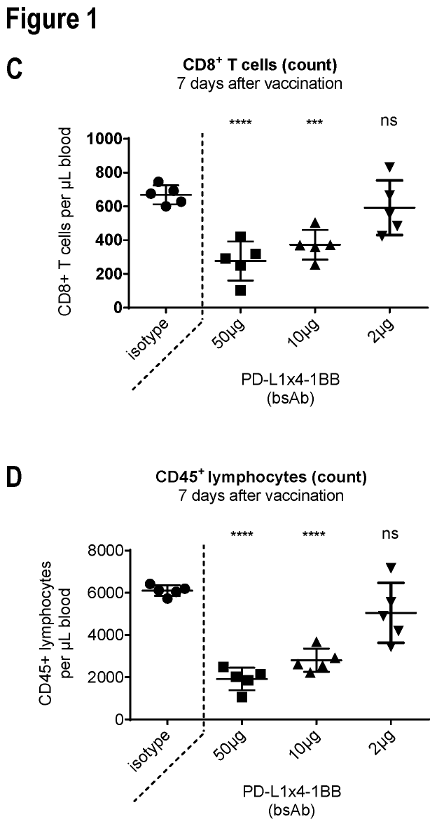

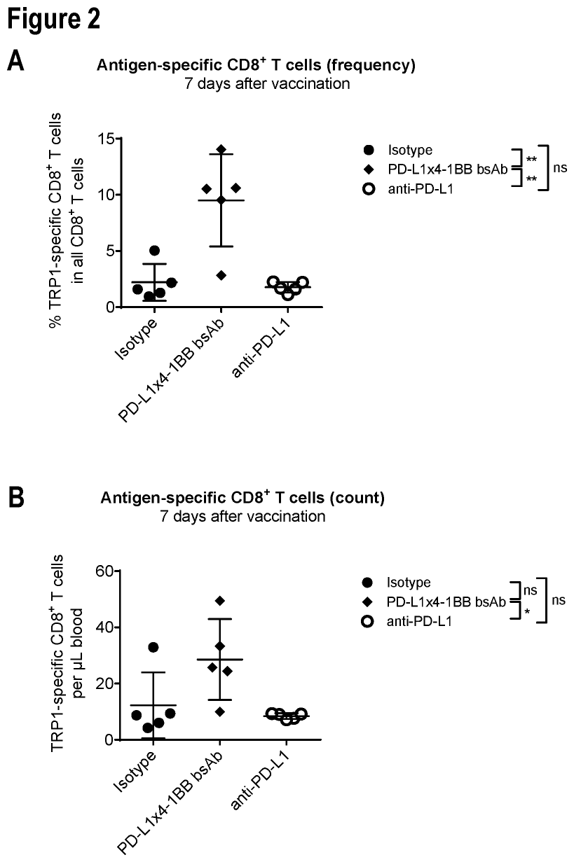

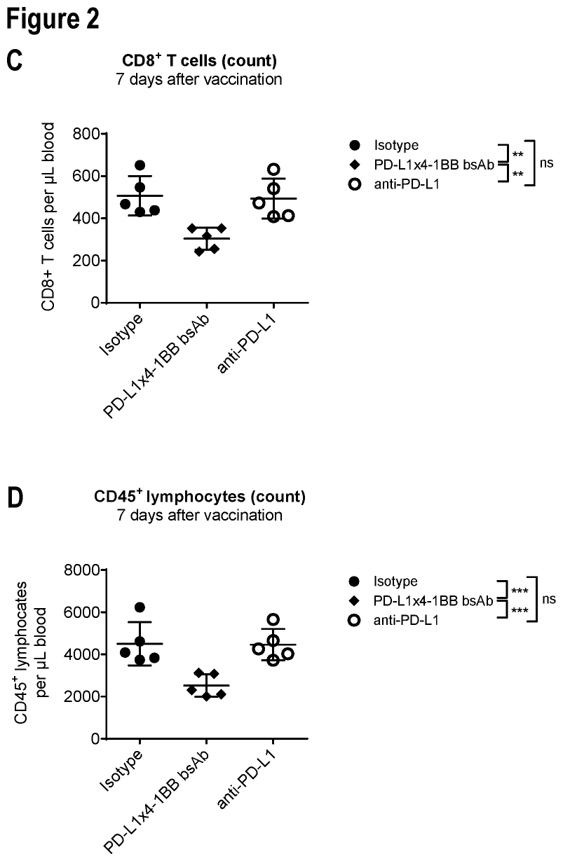

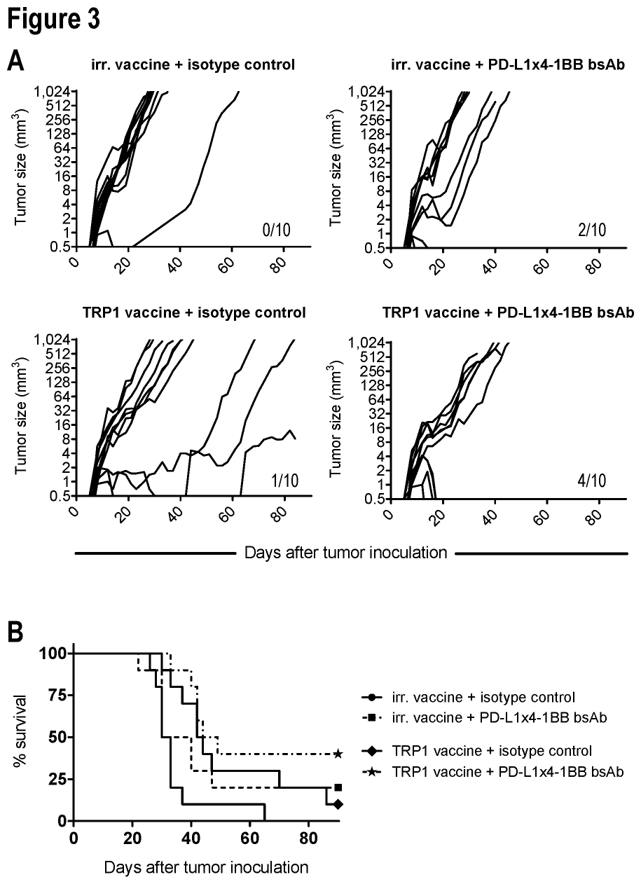

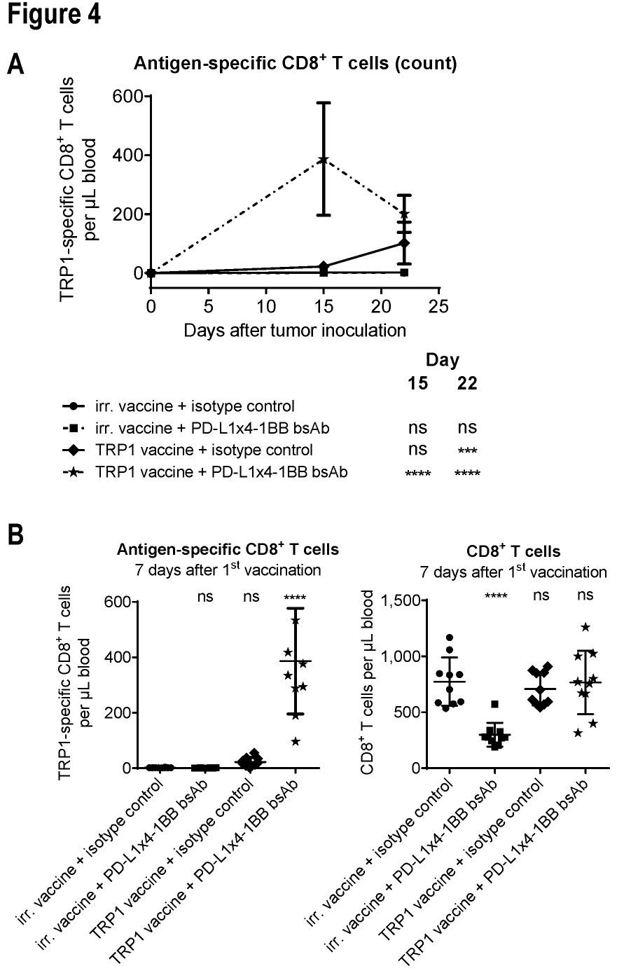

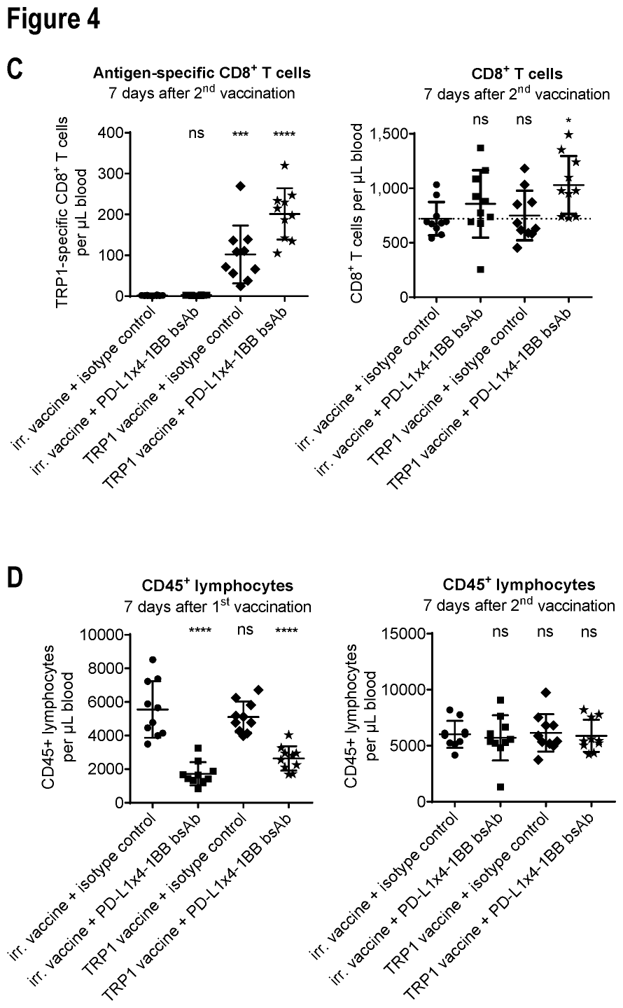

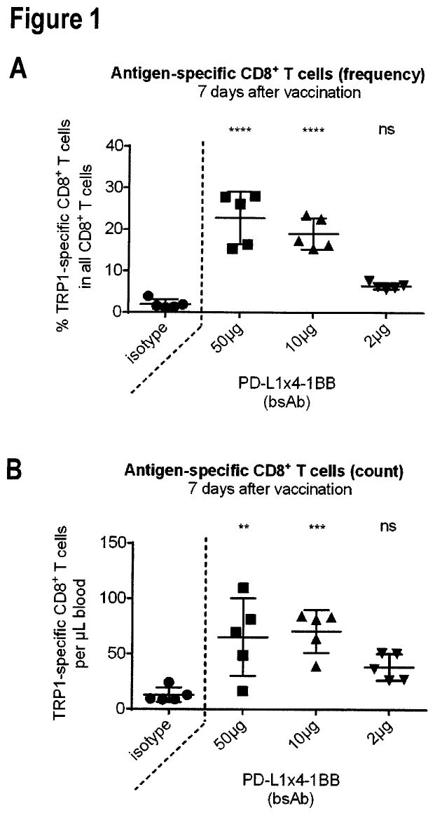

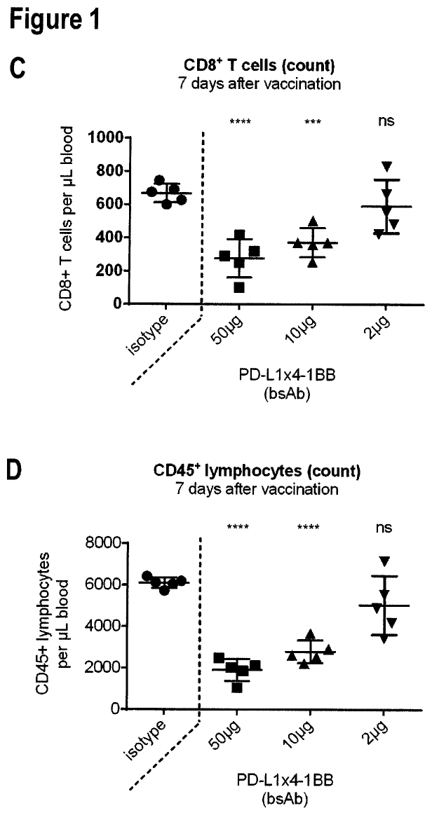

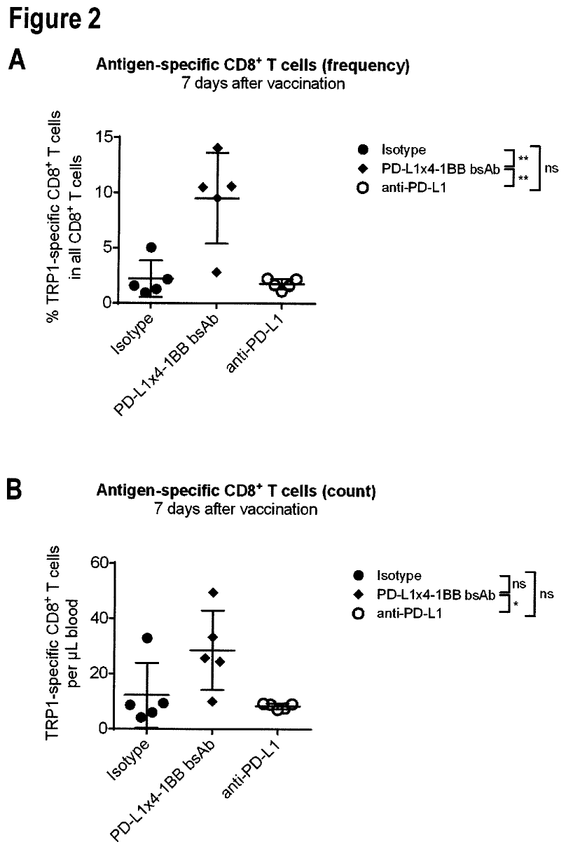

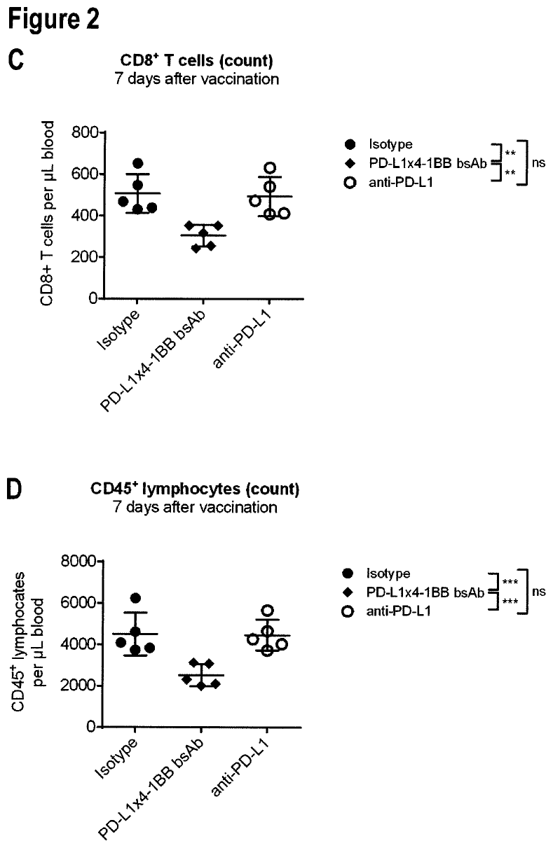

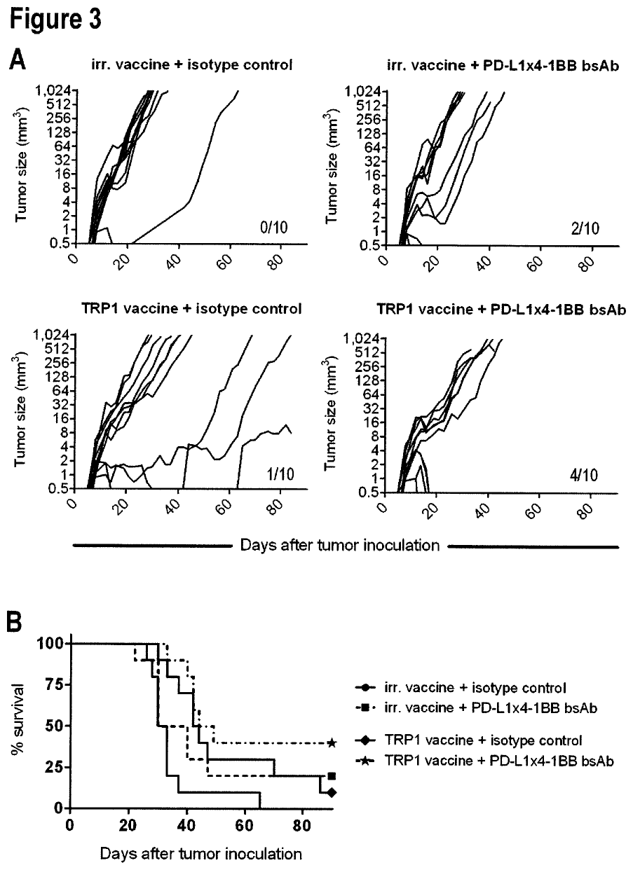

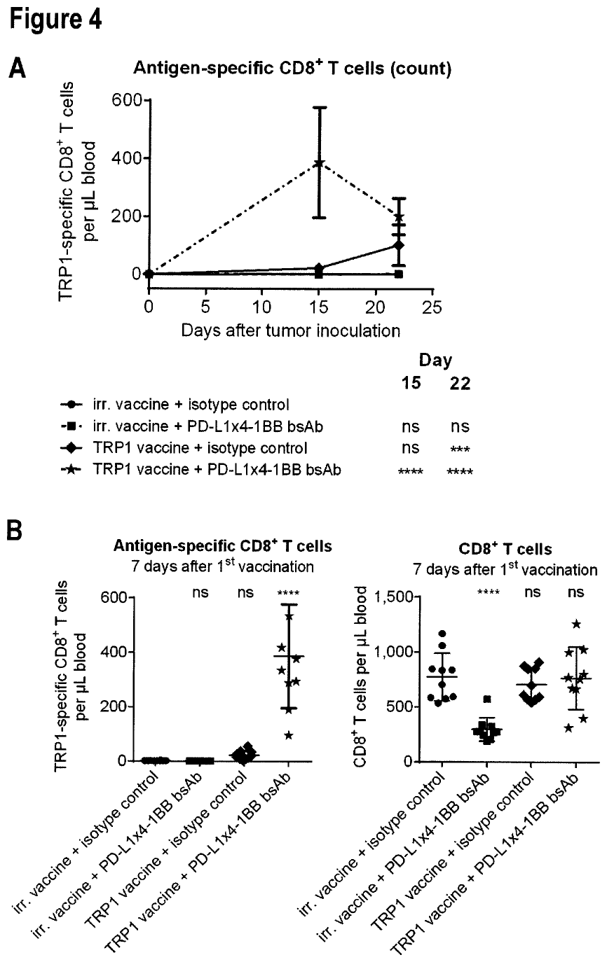

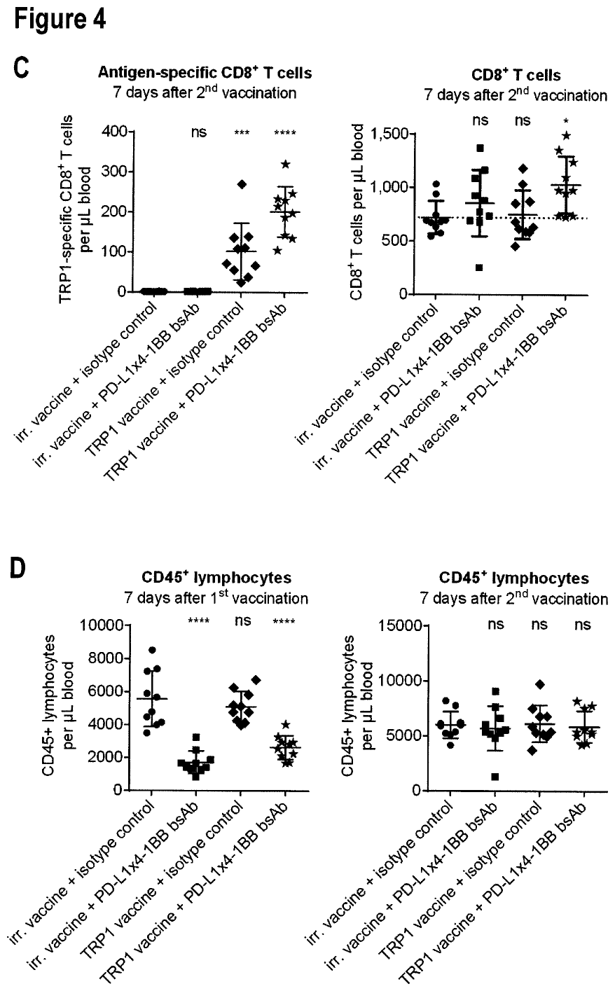

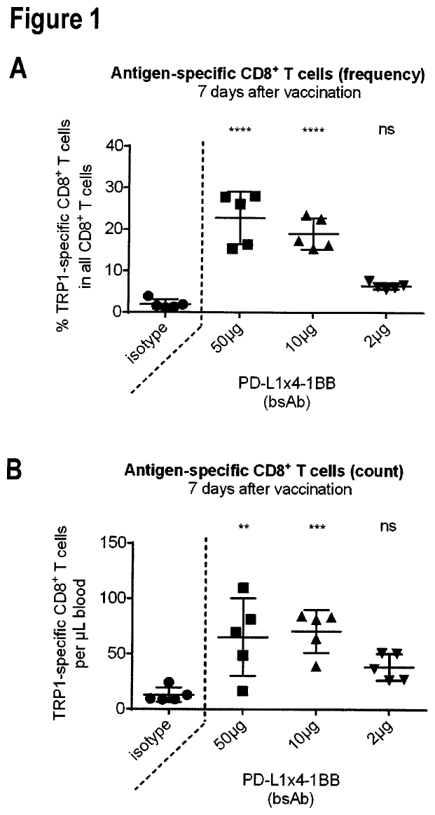

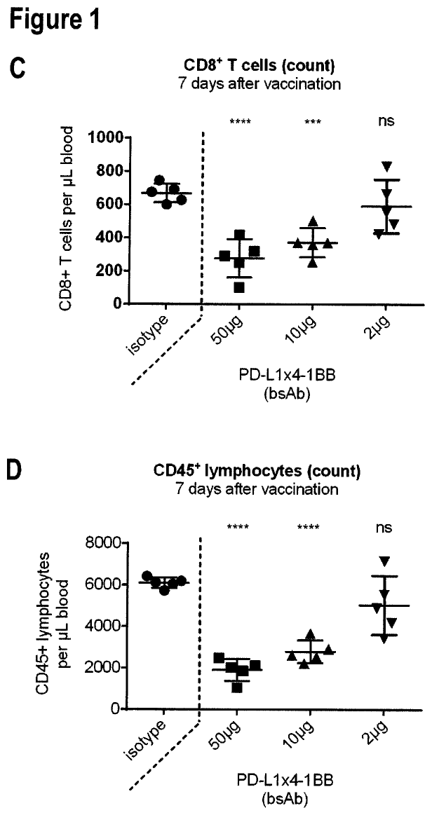

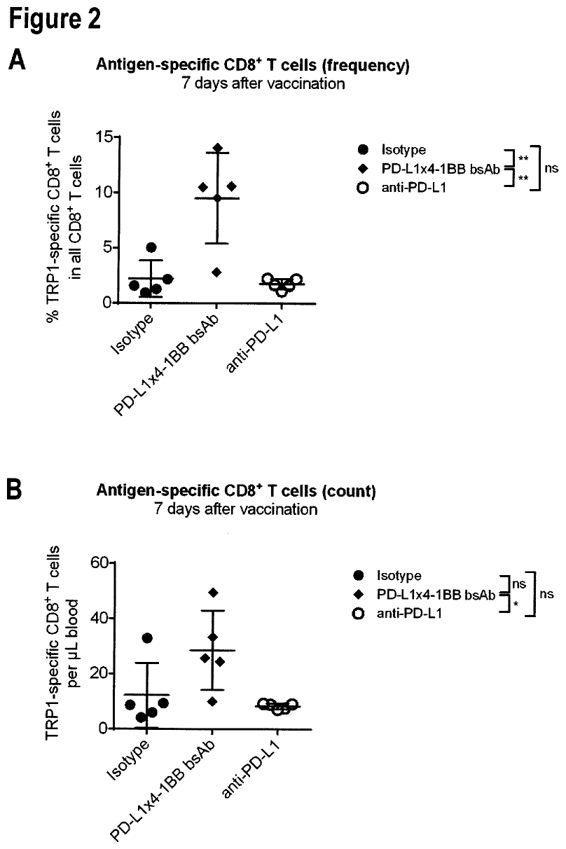

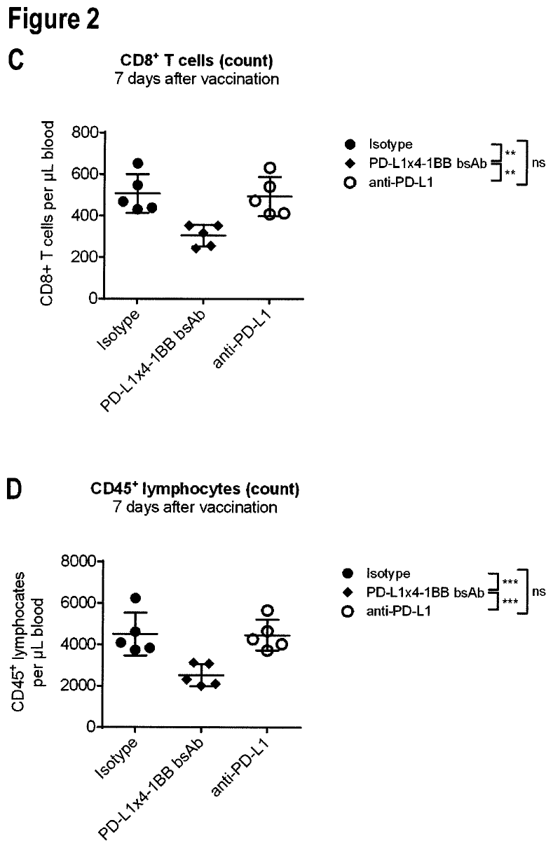

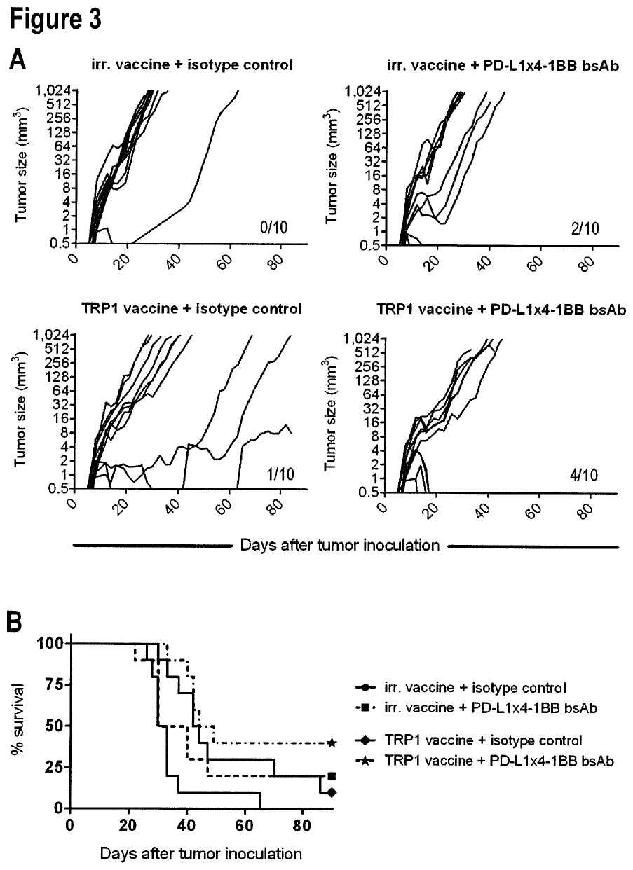

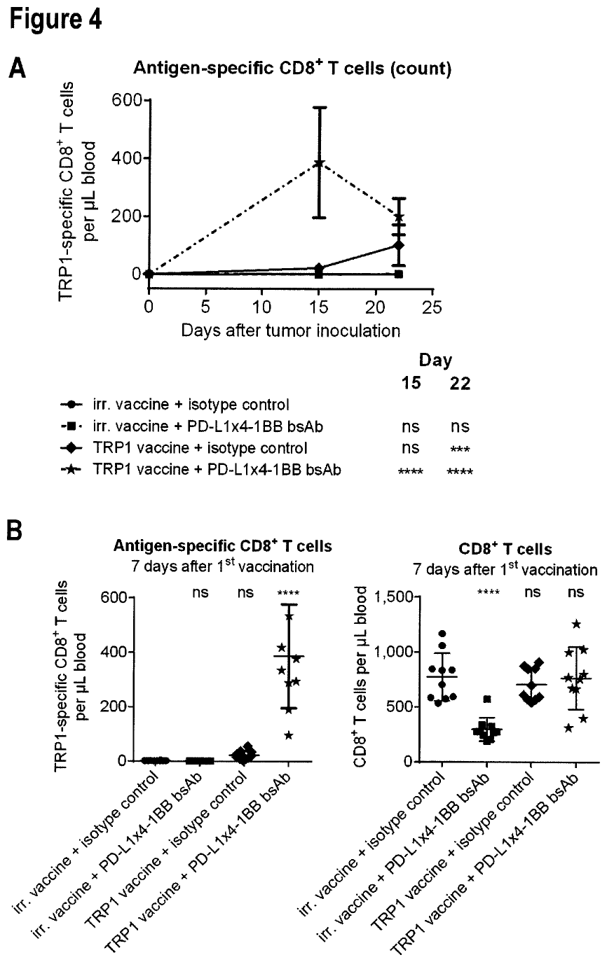

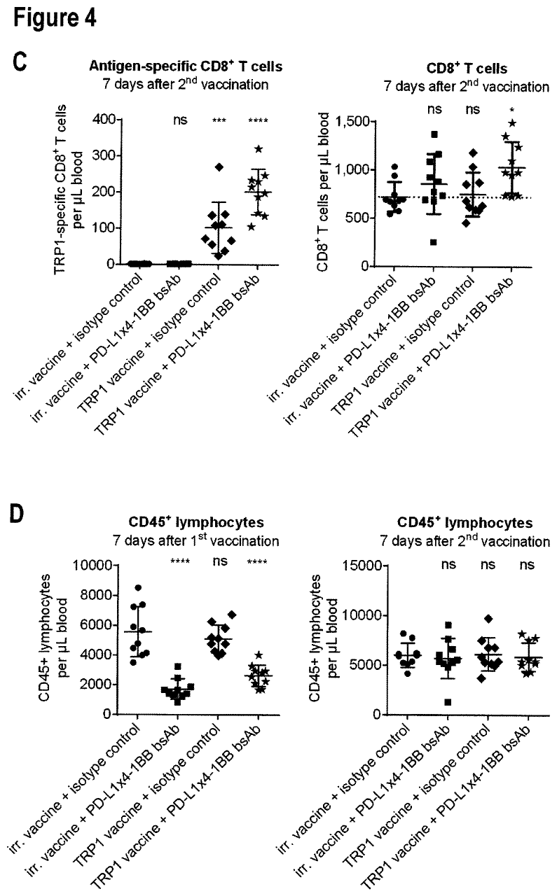

While TRH vaccination alone resulted in a weak TRH-specific CD8+ T-cell response (2.00±0.99% TRH-specific CD8+ T cells in all CD8+ T cells), the combination of TRH vaccine with PD-L1x4-1BB bsAb led to a strong and significant expansion of TRP1-specific CD8+ T cells, resulting in a frequency of 22.79±5.68% (50 pg/mouse PD-L1x4-1BB bsAb) TRP1-reactive CD8+ cells in all CD8+ T cells (FIG. 1A, Table 1). Due to the observed onset of lymphopenia in PD-L1x4-1BB bsAb treated animals (FIG. 1C and D; as previously described for 4-1BB agonist antibodies by Niu, L. et al, J Immuno) 178, 4194-4213 (2007)), the induced changes in TRP1-specific CD8+ T cell absolute count in between PD-L1x4-1BB bsAb treated cohorts and the control cohort were not as prominent as for the frequencies but still significant with 13.12±5.92 (isotype control) versus 65.48±31.29 (50 pg/mouse PD-L1x4-1BB bsAb) TRP1-specific CD8+ T cells per pl blood (FIG. 1B, Table 1). The effect of PD-L1x4-1BB bsAb treatment on TRP1-Tetramer-positive CD8+ T-cell frequencies as well as absolute counts in peripheral blood was relatively stable or even slightly more pronounced when the dose was reduced to 10 pg antibody/mouse (19.03±3.36% vs. 25.82±1.86% of all CD8+ T cells and 70.91±17.31 vs. 113.85±17.91 TRP1-specific CD8+ T cells per pl blood, for the 50 pg/mouse and 10 pg/mouse cohort, respectively). Important to note, even at the lowest dose tested (2 pg/mouse), a considerable increase of TRP1-specific CD8+ T cells per μl blood was observed compared to the isotype control.

TABLE 1
Absolute and relative TRP1-specific CD8⁺
T cell response in peripheral blood at day 8

| | | TRP1-reactive | TRP1-reactive |
|---|---|---|---|
| | | CD8⁺ T-cell | CD8⁺ T-cell |
| Combination | Antibody | frequency [% of all | count |
| partner | concentration | CD8⁺ T cells] | [per μL blood] |
| Isotype control | 50 μg/mouse | 2.00 ± 0.99 | 13.12 ± 5.92 |
| PD-L1x4-1BB | 50 μg/mouse | 22.79 ± 5.68 | 65.48 ± 31.29 |
| bsAb | 10 μg/mouse | 19.03 ± 3.36 | 70.91 ± 17.31 |
| | 2 μg/mouse | 6.50 ± 0.68 | 38.73 ± 10.77 |

Data is shown as mean ± standard deviation for n = 3 animals/cohort

### Example 2

### Mouse-Reactive PD-L1x4-1BB Bispecific Antibody is Superior to Anti-PD-L1 mAb In Amplifying Therapeutic RNA-Vaccine Induced Antigen-Specific T-Cell Responses

In order to compare the effect of a mouse-reactive PD-L1x4-1BB bispecific antibody (bsAb) and a classical anti-PD-L1 checkpoint-blockade monoclonal antibody (mAb) on RNA-vaccine induced T-cell responses in vivo, C57BL/6 mice (n=5 per group) were vaccinated i.v. with 20 pg liposomal formulated TRP1 RNA (TRP1 RNA-LPX) on day 1 and subsequently dosed intraperitoneally (i.p.) with either 50 pg mouse-reactive PD-L1x4-1BB bsAb or anti-PD-L1 mAb (clone MPDL3280A) per mouse on day 2. The control group received the TRP1 RNA-vaccine in combination with 50 μg of isotype control antibody. Lymphocyte subsets and TRP1-specific CD8+ T-cell responses in peripheral blood were determined on day 8 via flow cytometry (BD FACSCelesta ^{TM}, BD Biosciences) as described in Example 1.

TRP1 vaccination alone resulted in a weak TRP1-specific CD8+ T-cell response that could not be amplified by a combination of RNA-vaccine and andi-PD-L1 antibody (2.21±1.47% vs. 1.78±0.42% TRP1-specific CD8+ T cells in all CD8+ T cells, respectively). In contrast, the combination of TRP1 vaccine with PD-L1x4-1BB bsAb led to a significant expansion of TRP1-specific CD8+ T cells compared to both the isotype control and the anti-PD-L1 treated cohorts, resulting in a frequency of 9.51±3.67% TRP1-reactive CD8+ cells in all CD8+ T cells (FIG. 2A, Table 2). Due to the observed onset of lymphopenia in PD-L1x4-1BB bsAb treated animals (FIG. 2C and D; as previously described for 4-1BB agonist antibodies by Niu, L. et al, J Immunol 178, 4194-4213 (2007)), the induced changes in TRP1-specific CD8+ T cell absolute count in between the PD-L1x4-1BB bsAb treated cohort and the anti-PD-L1 as well as control cohorts were not as prominent as for the frequencies. However, changes were still significant when comparing the PD-L1x4-1BB bsAb and anti-PD-L1 treated cohorts with 28.59±12.88 (PD-L1x4-1BB bsAb) versus 8.45±0.90 (anti-PD-L1 mAb) TRP1-specific CD8+ T cells per pl blood (FIG. 2B, Table 2).

TABLE 2
Absolute and relative TRP1-specific CD8⁺
T cell response in peripheral blood at day 8

| | TRP1-reactive | |
|---|---|---|
| | CD8⁺ T-cell | TRP1-reactive CD8⁺ |
| | frequency [% of all | T-cell count |
| Combination partner | CD8⁺ T cells] | [per μL blood] |
| Isotype control | 2.21 ± 1.47 | 12.25 ± 10.49 |
| PD-L1x4-1BB bsAb | 9.51 ± 3.67 | 28.59 ± 12.88 |
| anti-PD-L1 mAb | 1.78 ± 0.42 | 8.45 ± 0.90 |

Data is shown as mean ± standard deviation for n = 3 animals/cohort

### Example 3: The Mouse-Reactive PD-L1x4-1BB Bispecific Antibody Boosts Anti-Tumoral Immunity In Combination with a Therapeutic RNA-Vaccine in the Murine Melanoma Model B16

In a next step, we characterized the potency of the mouse-reactive PD-L1x4-1BB bsAb to improve therapeutic anti-tumoral efficacy of an RNA vaccine in vivo. C57BL/6 mice were inoculated subcutaneously (s.c.) with 3x10⁵ B16-F10 melanoma cells and vaccinated i.v. at days 8, 15 and 22 with 20 Ξg TRP1 RNA-LPX or irrelevant RNA-LPX as control. Both vaccinated cohorts were treated i.p. with 50 Ξg of either PD-L1x4-1BB bsAb or an isotype control antibody at days 9, 12, 16, 19, 23 and 26, resulting in four treatment groups with n=10 animals per group. Blood lymphocyte subsets and TRP1-specific T-cell responses were determined seven days after the first two treatments (day 15 and 22) via flow cytometry as described in Example 1. Anti-tumor efficacy was determined as tumor growth inhibition in the test groups compared to the control group and overall survival during an observation period of up to day 90 after tumor inoculation.

Compared to the control group treated with isotype control and irrelevant RNA vaccine, TRP1 vaccination alone demonstrated moderate therapeutic efficacy with 3/10 initial responders of which ⅔ eventually succumbed to relapse. The remaining animals of the control cohort showed a modest tendency for tumor growth reduction and increased median survival (FIG. 3A, B). In comparison, PD-L1x4-1BB bsAb monotherapy resulted in complete rejection of tumors in 2/10 (20%) animals, however, the remaining animals showed no increased median survival. Most importantly, PD-L1x4-1BB bsAb treatment benefited from simultaneous TRP1 vaccination, resulting in further tumor growth reduction and enhanced survival compared to each individual therapy. Of the animals treated with the combination of PD-L1x4-1BB bsAb and TRP1 vaccine, 4/10 (40%) showed a complete rejection of their tumors.

Treatment with PD-L1x4-1BB bsAb alone did not induce TRP1-specific CD8+ T cells, while TRP1 vaccination resulted in a weak TRP1-specific CD8+ T-cell response after the first vaccination (22.41±15.27 TRP1-specific CD8+ T cells) that can be significantly boosted by the second vaccination (FIG. 4A-C; 102.01±67.17 TRP1-specific CD8+ T cells). The combination of PD-L1x4-1BB bsAb with the TRP1 vaccine strongly boosted TRP1-specific CD8+ T-cell numbers already after the first vaccination with >10-fold higher numbers of TRP1-specific CD8+ T cells in peripheral blood being detectable when compared to TRP1 vaccination alone (386.60±181.04 vs. 22.41±15.27). After the second TRP1 vaccination and antibody treatment cycle, TRP1-specific CD8+ T-cell numbers drop to 201.32±59.73, however, still being superior compared to the TRP1-specific T cell response in TRP1 vaccination monotherapy. As observed before (Example 1 and 2, FIG. 1C-D, FIG. 2C-D), PD-L1x4-1BB bsAb treatment resulted in transient lymphopenia apparent in both PD-L1x4-1BB bsAb treated cohorts at day 7 after 1st vaccination. Importantly, lymphocyte count is back to baseline 7 days after the 2^{nd} vaccination (FIG. 4D).

In summary, PD-L1x4-1BB bsAb is to some extent effective as monotherapy also in a lowly immunogenic tumor model and synergizes with T-cell vaccination by expanding vaccine-induced T-cell responses.

1. A method for treating a subject comprising administering to the subject:
a. a peptide or protein comprising an epitope for inducing an immune response against an antigen in the subject or a polynucleotide encoding the peptide or protein; and
b. a binding agent comprising a first antigen-binding region binding to PD-L1 and a second antigen-binding region binding to CD137, or a polynucleotide encoding the binding agent.

2. The method of claim 1, wherein the subject is a human.

3. The method of claim 1 or 2, wherein the PD-L1 is human PD-L1 and/or the CD137 is human CD137.

4. The method of any one of claims 1 to 3, wherein said first antigen-binding region binding to PD-L1 inhibits the binding of human PD-L1 to human PD-1

5. The method of any one of claims 1 to 4, wherein said first antigen-binding region binding to PD-L1 comprises a heavy chain variable region (VH) comprising a HCDR3 having the sequence as set forth in SEQ ID NO: 13.

6. The method of any one of claims 1 to 5, wherein said first antigen-binding region binding to PD-L1 comprises a heavy chain variable region (VH) comprising a HCDR2 having the sequence as set forth in SEQ ID NO: 12.

7. The method of any one of claims 1 to 6, wherein said first antigen-binding region binding to PD-L1 comprises a heavy chain variable region (VH) comprising a HCDR1 having the sequence as set forth in SEQ ID NO: 11.

8. The method of any one of claims 1 to 7, wherein said first antigen-binding region binding to PD-L1 comprises a heavy chain variable region (VH) comprising a HCDR1, HCDR2, and HCDR3 sequence, wherein the HCDR1, HCDR2 and HCDR3 sequence comprises the sequence as set forth in SEQ ID NO:
11, 12, and 13, respectively.

9. The method of any one of claims 1 to 8, wherein said first antigen-binding region binding to PD-L1 comprises a light chain variable region (VL) comprising a LCDR3 having the sequence as set forth in SEQ ID NO: 16.

10. The method of any one of claims 1 to 9, wherein said first antigen-binding region binding to PD-L1 comprises a light chain variable region (VL) comprising a LCDR2 having the sequence DDN.

11. The method of any one of claims 1 to 10, wherein said first antigen-binding region binding to PD-L1 comprises a light chain variable region (VL) comprising a LCDR1 having the sequence as set forth in SEQ ID NO: 15.

12. The method of any one of claims 1 to 11, wherein said first antigen-binding region binding to PD-L1 comprises a light chain variable region (VL) comprising a LCDR1, LCDR2, and LCDR3 sequence, wherein the LCDR1, LCDR2 and LCDR3 sequence comprises the sequence as set forth in SEQ ID NO:
15, DDN, and 16, respectively.

13. The method of any one of claims 1 to 12, wherein said first antigen-binding region binding to PD-L1 comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises the sequence as set forth in SEQ ID NO: 11, 12, and 13, respectively, and the LCDR1, LCDR2 and LCDR3 sequence comprises the sequence as set forth in SEQ ID NO: 15, DDN, and 16, respectively.

14. The method of any one of claims 1 to 13, wherein said first antigen-binding region binding to PD-L1 comprises a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 10.

15. The method of any one of claims 1 to 14, wherein said first antigen-binding region binding to PD-L1 comprises a heavy chain variable region (VH), wherein the VH comprises the sequence as set forth in SEQ ID NO: 10.

16. The method of any one of claims 1 to 15, wherein said first antigen-binding region binding to PD-L1 comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 14.

17. The method of any one of claims 1 to 16, wherein said first antigen-binding region binding to PD-L1 comprises a light chain variable region (VL), wherein the VL comprises the sequence as set forth in SEQ ID NO: 14.

18. The method of any one of claims 1 to 17, wherein said first antigen-binding region binding to PD-L1 comprises a heavy chain variable region (VH) and a light chain variable region (VL), wherein the VH comprises the sequence as set forth in SEQ ID NO: 10 and the VL comprises the sequence as set forth in SEQ ID NO: 14.

19. The method of any one of claims 1 to 18, wherein said first antigen-binding region binding to PD-L1 comprises heavy and light chain variable regions of an antibody which competes for PD-L1 binding with and/or has the specificity for PD-L1 of an antibody comprising a heavy chain variable region (VH) and/or a light chain variable region (VL) as set forth in any one of claims 5 to 18.

20. The method of any one of claims 1 to 19, wherein said second antigen-binding region binding to CD137 comprises a heavy chain variable region (VH) comprising a HCDR3 having the sequence as set forth in SEQ ID NO: 4.

21. The method of any one of claims 1 to 20, wherein said second antigen-binding region binding to CD137 comprises a heavy chain variable region (VH) comprising a HCDR2 having the sequence as set forth in SEQ ID NO: 3.

22. The method of any one of claims 1 to 21, wherein said second antigen-binding region binding to CD137 comprises a heavy chain variable region (VH) comprising a HCDR1 having the sequence as set forth in SEQ ID NO: 2.

23. The method of any one of claims 1 to 22, wherein said second antigen-binding region binding to CD137 comprises a heavy chain variable region (VH) comprising a HCDR1, HCDR2, and HCDR3 sequence, wherein the HCDR1, HCDR2 and HCDR3 sequence comprises the sequence as set forth in SEQ ID NO: 2, 3, and 4, respectively.

24. The method of any one of claims 1 to 23, wherein said second antigen-binding region binding to CD137 comprises a light chain variable region (VL) comprising a LCDR3 having the sequence as set forth in SEQ ID NO: 7.

25. The method of any one of claims 1 to 24, wherein said second antigen-binding region binding to CD137 comprises a light chain variable region (VL) comprising a LCDR2 having the sequence GAS.

26. The method of any one of claims 1 to 25, wherein said second antigen-binding region binding to CD137 comprises a light chain variable region (VL) comprising a LCDR1 having the sequence as set forth in SEQ ID NO: 6.

27. The method of any one of claims 1 to 26, wherein said second antigen-binding region binding to CD137 comprises a light chain variable region (VL) comprising a LCDR1, LCDR2, and LCDR3 sequence, wherein the LCDR1, LCDR2 and LCDR3 sequence comprises the sequence as set forth in SEQ ID NO: 6, GAS, and 7, respectively.

28. The method of any one of claims 1 to 27, wherein said second antigen-binding region binding to CD137 comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises the sequence as set forth in SEQ ID NO: 2, 3, and 4, respectively, and the LCDR1, LCDR2 and LCDR3 sequence comprises the sequence as set forth in SEQ ID NO: 6, GAS, and 7, respectively.

29. The method of any one of claims 1 to 28, wherein said second antigen-binding region binding to CD137 comprises a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 1 or 8.

30. The method of any one of claims 1 to 29, wherein said second antigen-binding region binding to CD137 comprises a heavy chain variable region (VH), wherein the VH comprises the sequence as set forth in SEQ ID NO: 1 or 8.

31. The method of any one of claims 1 to 30, wherein said second antigen-binding region binding to CD137 comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 5 or 9.

32. The method of any one of claims 1 to 31, wherein said second antigen-binding region binding to CD137 comprises a light chain variable region (VL), wherein the VL comprises the sequence as set forth in SEQ ID NO: 5 or 9.

33. The method of any one of claims 1 to 32, wherein said second antigen-binding region binding to CD137 comprises a heavy chain variable region (VH) and a light chain variable region (VL), wherein the VH comprises the sequence as set forth in SEQ ID NO: 1 and the VL comprises the sequence as set forth in SEQ ID NO: 5.

34. The method of any one of claims 1 to 32, wherein said second antigen-binding region binding to CD137 comprises a heavy chain variable region (VH) and a light chain variable region (VL), wherein the VH comprises the sequence as set forth in SEQ ID NO: 8 and the VL comprises the sequence as set forth in SEQ ID NO: 9.

35. The method of any one of claims 1 to 19, wherein said second antigen-binding region binding to CD137 comprises a heavy chain variable region (VH) comprising a HCDR3 having the sequence as set forth in SEQ ID NO: 27.

36. The method of any one of claims 1 to 19, and 35, wherein said second antigen-binding region binding to CD137 comprises a heavy chain variable region (VH) comprising a HCDR2 having the sequence as set forth in SEQ ID NO: 26.

37. The method of any one of claims 1 to 19, 35, and 36, wherein said second antigen-binding region binding to CD137 comprises a heavy chain variable region (VH) comprising a HCDR1 having the sequence as set forth in SEQ ID NO: 25.

38. The method of any one of claims 1 to 19, and 35 to 37, wherein said second antigen-binding region binding to CD137 comprises a heavy chain variable region (VH) comprising a HCDR1, HCDR2, and HCDR3 sequence, wherein the HCDR1, HCDR2 and HCDR3 sequence comprises the sequence as set forth in SEQ ID NO: 25, 26, and 27, respectively.

39. The method of any one of claims 1 to 19, and 35 to 38, wherein said second antigen-binding region binding to CD137 comprises a light chain variable region (VL) comprising a LCDR3 having the sequence as set forth in SEQ ID NO: 30.

40. The method of any one of claims 1 to 19, and 35 to 39, wherein said second antigen-binding region binding to CD137 comprises a light chain variable region (VL) comprising a LCDR2 having the sequence SAS.

41. The method of any one of claims 1 to 19, and 35 to 40, wherein said second antigen-binding region binding to CD137 comprises a light chain variable region (VL) comprising a LCDR1 having the sequence as set forth in SEQ ID NO: 29.

42. The method of any one of claims 1 to 19, and 35 to 41, wherein said second antigen-binding region binding to CD137 comprises a light chain variable region (VL) comprising a LCDR1, LCDR2, and LCDR3 sequence, wherein the LCDR1, LCDR2 and LCDR3 sequence comprises the sequence as set forth in SEQ ID NO: 29, SAS, and 30, respectively.

43. The method of any one of claims 1 to 19, and 35 to 42, wherein said second antigen-binding region binding to CD137 comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises the sequence as set forth in SEQ ID NO: 25, 26, and 27, respectively, and the LCDR1, LCDR2 and LCDR3 sequence comprises the sequence as set forth in SEQ ID NO: 29, SAS, and 30, respectively.

44. The method of any one of claims 1 to 19, and 35 to 43, wherein said second antigen-binding region binding to CD137 comprises a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 24.

45. The method of any one of claims 1 to 19, and 35 to 44, wherein said second antigen-binding region binding to CD137 comprises a heavy chain variable region (VH), wherein the VH comprises the sequence as set forth in SEQ ID NO: 24.

46. The method of any one of claims 1 to 19, and 35 to 45, wherein said second antigen-binding region binding to CD137 comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 28.

47. The method of any one of claims 1 to 19, and 35 to 46, wherein said second antigen-binding region binding to CD137 comprises a light chain variable region (VL), wherein the VL comprises the sequence as set forth in SEQ ID NO: 28.

48. The method of any one of claims 1 to 19, and 35 to 47, wherein said second antigen-binding region binding to CD137 comprises a heavy chain variable region (VH) and a light chain variable region (VL), wherein the VH comprises the sequence as set forth in SEQ ID NO: 24 and the VL comprises the sequence as set forth in SEQ ID NO: 28.

49. The method of any one of claims 1 to 48, wherein said second antigen-binding region binding to CD137 comprises heavy and light chain variable regions of an antibody which competes for CD137 binding with and/or has the specificity for CD137 of an antibody comprising a heavy chain variable region and/or a light chain variable region as set forth in any one of claims 20 to 48.

50. The method of any one of claims 1 to 49, wherein the binding agent is in the format of a full-length antibody.

51. The method of any one of claims 1 to 49, wherein the binding agent is in the format of an antibody fragment.

52. The method of any one of claims 1 to 51, wherein the binding agent is a multispecific antibody such as a bispecific antibody.

53. The method of any one of claims 1 to 52, wherein the peptide or protein comprising an epitope for inducing an immune response against an antigen in the subject or the polynucleotide encoding the peptide or protein and the binding agent or the polynucleotide encoding the binding agent are administered sequentially.

54. The method of any one of claims 1 to 53, wherein the binding agent or the polynucleotide encoding the binding agent is administered following administration of the peptide or protein comprising an epitope for inducing an immune response against an antigen in the subject or the polynucleotide encoding the peptide or protein.

55. The method of any one of claims 1 to 54, wherein the binding agent or the polynucleotide encoding the binding agent is administered 6 hours or later, 12 hours or later or 24 hours or later following administration of the peptide or protein comprising an epitope for inducing an immune response against an antigen in the subject or the polynucleotide encoding the peptide or protein.

56. The method of any one of claims 1 to 55, wherein the binding agent or the polynucleotide encoding the binding agent is administered between 12 hours and 48 hours following administration of the peptide or protein comprising an epitope for inducing an immune response against an antigen in the subject or the polynucleotide encoding the peptide or protein.

57. The method of any one of claims 1 to 56, wherein the polynucleotide encoding the peptide or protein comprising an epitope for inducing an immune response against an antigen in the subject and/or the polynucleotide encoding the binding agent is RNA.

58. The method of any one of claims 1 to 57, comprising administering to the subject:
a. RNA encoding the peptide or protein comprising an epitope for inducing an immune response against an antigen in the subject; and
b. the binding agent.

59. The method of any one of claims 1 to 58, wherein administering the binding agent or the polynucleotide encoding the binding agent increases the number of CD8 positive T cells which are specific for the antigen or cells expressing the antigen.

60. The method of any one of claims 1 to 59, which is a method for inducing an immune response in the subject.

61. The method of any one of claims 1 to 60, which is a method for inducing an immune response against the antigen or cells expressing the antigen in the subject.

62. The method of any one of claims 1 to 61, which is a method for treating or preventing cancer in the subject, wherein the antigen is a tumor-associated antigen.

63. A medical preparation, comprising:
a. a peptide or protein comprising an epitope for inducing an immune response against an antigen in a subject, or a polynucleotide encoding the peptide or protein; and
b. a binding agent comprising a first antigen-binding region binding to PD-L1 and a second antigen-binding region binding to CD137, or a polynucleotide encoding the binding agent.

64. The medical preparation of claim 63, comprising:
a. RNA encoding the peptide or protein comprising an epitope for inducing an immune response against an antigen in a subject; and
b. the binding agent.

65. The medical preparation of claim 63 or 64, which is a kit.

66. The medical preparation of any one of claims 63 to 65, which comprises each component a. and b. in a separate container.

67. The medical preparation of any one of claims 63 to 66, further comprising instructions for use of the medical preparation for treating or preventing cancer, wherein the antigen is a tumor-associated antigen.

68. The medical preparation of any one of claims 63 to 67 for pharmaceutical use.

69. The medical preparation of claim 68, wherein the pharmaceutical use comprises a therapeutic or prophylactic treatment of a disease or disorder.

70. The medical preparation of any one of claims 63 to 69 for use in a method for treating or preventing cancer in a subject, wherein the antigen is a tumor-associated antigen.


SEQUENCE LISTING (30 sequences)

SEQ ID NO: 1 - protein, 114 aa, Artificial Sequence
  Heavy chain variable region (VH)
QSLEESGGRLVTPGTPLTLTCTVSGFSLNDYWMSWVRQAPGKGLEWIGYIDVGGSLYYASWAKGRFTISRTSTTVDLKMTSLTTEDTATYFCARGGLTYGFDLWGPGTLVTVSS

SEQ ID NO: 2 - protein, 8 aa, Artificial Sequence
  Complementarity determining region (CDR)
GFSLNDYW

SEQ ID NO: 3 - protein, 7 aa, Artificial Sequence
  Complementarity determining region (CDR)
IDVGGSL

SEQ ID NO: 4 - protein, 11 aa, Artificial Sequence
  Complementarity determining region (CDR)
ARGGLTYGFDL

SEQ ID NO: 5 - protein, 110 aa, Artificial Sequence
  Light chain variable region (VL)
DIVMTQTPASVSEPVGGTVTINCQASEDISSYLAWYQQKPGQRPKRLIYGASDLASGVPSRFSASGSGTEYALTISDLESADAATYYCHYYATISGLGVAFGGGTEVVVK

SEQ ID NO: 6 - protein, 6 aa, Artificial Sequence
  Complementarity determining region (CDR)
EDISSY

SEQ ID NO: 7 - protein, 12 aa, Artificial Sequence
  Complementarity determining region (CDR)
HYYATISGLGVA

SEQ ID NO: 8 - protein, 117 aa, Artificial Sequence
  Heavy chain variable region (VH)
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSS

SEQ ID NO: 9 - protein, 110 aa, Artificial Sequence
  Light chain variable region (VL)
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIK

SEQ ID NO: 10 - protein, 121 aa, Artificial Sequence
  Heavy chain variable region (VH)
EVQLLEPGGGLVQPGGSLRLSCEASGSTFSTYAMSWVRQAPGKGLEWVSGFSGSGGFTFYADSVRGRFTISRDSSKNTLFLQMSSLRAEDTAVYYCAIPARGYNYGSFQHWGQGTLVTVSS

SEQ ID NO: 11 - protein, 8 aa, Artificial Sequence
  Complementarity determining region (CDR)
GSTFSTYA

SEQ ID NO: 12 - protein, 8 aa, Artificial Sequence
  Complementarity determining region (CDR)
FSGSGGFT

SEQ ID NO: 13 - protein, 14 aa, Artificial Sequence
  Complementarity determining region (CDR)
AIPARGYNYGSFQH

SEQ ID NO: 14 - protein, 108 aa, Artificial Sequence
  Light chain variable region (VL)
SYVLTQPPSVSVAPGQTARITCGGNNIGSKSVHWYQQKPGQAPVLVVYDDNDRPSGLPERFSGSNSGNTATLTISRVEAGDEADYYCQVWDSSSDHVVFGGGTKLTVL

SEQ ID NO: 15 - protein, 6 aa, Artificial Sequence
  Complementarity determining region (CDR)
NIGSKS

SEQ ID NO: 16 - protein, 11 aa, Artificial Sequence
  Complementarity determining region (CDR)
QVWDSSSDHVV

SEQ ID NO: 17 - protein, 330 aa, Artificial Sequence
  Heavy chain constant region (CH)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 18 - protein, 330 aa, Artificial Sequence
  Heavy chain constant region (CH)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 19 - protein, 107 aa, Artificial Sequence [sequence omitted]

SEQ ID NO: 20 - protein, 106 aa, Artificial Sequence
  Light chain constant region (CL)
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 22 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 23 - protein, 232 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 24 - protein, 119 aa, Artificial Sequence
  Heavy chain variable region (VH)
QSVEESGGRLVTPGTPLTLTCTASGFTISDFHVTWVRQAPGKGLEWIGTIITSASTTAYATWARGRFTISKSSTTVNLKIVSPTTEDTATYFCARSTYTDTSGYYFDFWGQGTLVTVSS

SEQ ID NO: 25 - protein, 8 aa, Artificial Sequence
  Complementarity determining region (CDR)
GFTISDFH

SEQ ID NO: 26 - protein, 8 aa, Artificial Sequence
  Complementarity determining region (CDR)
IITSASTT

SEQ ID NO: 27 - protein, 15 aa, Artificial Sequence
  Complementarity determining region (CDR)
ARSTYTDTSGYYFDF

SEQ ID NO: 28 - protein, 113 aa, Artificial Sequence
  Light chain variable region (VL)
AQVLTQTASPVSAAVGGTVIINCQSSQSIYNGNRLSWYQQKPGQPPKLLIYSASTLASGVSSRFKGSGSGTQFTLAISDVQSDDAATYYCLGSYDCDSADCFAFGGGTEVVVE

SEQ ID NO: 29 - protein, 8 aa, Artificial Sequence
  Complementarity determining region (CDR)
QSIYNGNR

SEQ ID NO: 30 - protein, 13 aa, Artificial Sequence
  Complementarity determining region (CDR)
LGSYDCDSADCFA